(19) United States
(12) Patent Application Publication
(10) Pub. No.: US 2024/0329041 A1
(43) Pub. Date: Oct. 3, 2024

(54) Biomarkers of Marine Invertebrate Stress

(71) Applicant: Rutgers, The State University of New Jersey, New Brunswick, NJ (US)

(72) Inventors: Debashish Bhattacharya, Princeton, NJ (US); Timothy G. Stephens, New Brunswick, NJ (US); Shrinivas Nandi, New Brunswick, NJ (US); Erin E. Chille, Highland Park, NJ (US)

(21) Appl. No.: 18/618,557

(22) Filed: Mar. 27, 2024

Related U.S. Application Data
(60) Provisional application No. 63/492,432, filed on Mar. 27, 2023.

(51) Int. Cl. G01N 33/543; G01N 33/68

(52) U.S. Cl. CPC G01N 33/54388; G01N 33/68; G01N 2333/43504; G01N 2333/90241; G01N 2333/90245; G01N 2333/904; G01N 2333/91017; G01N 2333/9125

(57) ABSTRACT

Provided herein are, in various embodiments, devices and compositions for detecting, monitoring, and/or treating environmental stress in, and/or selecting for propagation and/or protection, one or more marine invertebrates. In some embodiments of the disclosure, the devices and compositions provide for the detection, monitoring, treatment, and or selection of coral holobionts in response to thermal stress. In still further embodiments, the disclosure provides for the selection of coral holobionts for increased viability, variation, abundance, proliferation, fecundity, or a combination thereof. In some embodiments, the disclosure provides for mitigation against the loss of coral reefs due to climate change.

### RELATED APPLICATION(S)

This application claims the benefit of U.S. Provisional Application No. 63/492,432, filed on Mar. 27, 2023. The entire teachings of the above application(s) are incorporated herein by reference.

### BACKGROUND

The integration of multiple ‘omics’ (multi-omics) datasets is a promising avenue for answering many important and challenging questions in biology, particularly those relating to complex ecological systems such as coral reefs. Multi-omics was, however, developed using data from model organisms which have significant prior knowledge and resources available. It is unclear if multi-omics can be effectively applied to non-model organisms, such as coral holobionts, which house an assemblage of microbial partners and have not yet been widely studied using these approaches.

### SUMMARY

The present disclosure explores, in a model marine invertebrate, the emerging rice coral model Montipora capitata (M. capitata), how transcriptomic, proteomic, metabolomic, and microbiome amplicon datasets interact across the coral holobiont and how well their overall patterns correlate with thermal stress. The present disclosure shows that transcriptomic and proteomic data broadly capture the stress response of the coral, whereas the metabolome and microbiome datasets show patterns that likely reflect stochastic and homeostatic processes associated with each sample. These results provide a framework for interpreting multi-omics data generated from non-model systems, particularly those with complex biotic interactions among microbial partners. Further, analysis and interpretation of these results led to identification of marine invertebrate biomarkers of environmental stress.

In one aspect, the present disclosure provides a device for detecting environmental stress or an effect thereof in a sample from a marine invertebrate, wherein the device detects a biomarker.

In some embodiments, environmental stress comprises, involves, or is related to heat stress, thermal stress, temperature sensitivity-inducing stress, stress as a function of temperature, and the like. In some embodiments, the environmental stress comprises thermal stress.

In some embodiments, the effect of environmental stress comprises at least one effect selected from the group comprising: reduced health, increased stress, reduced viability, reduced variation, reduced abundance, reduced proliferation, and reduced fecundity.

In some embodiments, the marine invertebrate comprises a holobiont. In some embodiments, the marine invertebrate comprises coral or coral nubbins.

In some embodiments, the device comprises a test strip or a lateral flow immunoassay (LFIA).

In some embodiments, the biomarker is detected at a level at least 2-fold higher or lower in the sample compared to a control.

In some embodiments, the biomarker is selected from the group comprising: phenylalanine-4-hydroxylase, glycine N-methyltransferase, glyoxylate/hydroxypyruvate reductase, 2′-5′-oligoadenylate synthetase, 4-hydroxyphenylpyruvate dioxygenase, and homogentisate 1,2-dioxygenase.

In another aspect, the present disclosure provides for use of the device for detecting environmental stress or an effect thereof in a sample from a marine invertebrate for: monitoring health of a marine invertebrate; detecting environmental stress or an effect thereof in a marine invertebrate; detecting viability, variation, abundance, proliferation, or fecundity of a marine invertebrate; treating a marine invertebrate; identifying a marine invertebrate for propagation or protection; or a combination of any of the foregoing.

In some embodiments, treating a marine invertebrate comprises at least one intervention selected from the group comprising: prohibition of fishing, prohibition of swimming, and prohibition of boating.

In yet another aspect, the present disclosure provides a composition for use as a standard in detecting environmental stress or an effect thereof in a sample from a marine invertebrate, wherein the composition comprises one or more known amounts of one or more biomarkers.

In some embodiments, the environmental stress comprises thermal stress. In some embodiments, the effect of environmental stress is at least one effect selected from the group comprising: reduced health, increased stress, reduced viability, reduced variation, reduced abundance, reduced proliferation, and reduced fecundity.

In some embodiments, the marine invertebrate comprises a holobiont. In some embodiments, the marine invertebrate comprises coral or coral nubbins.

In some embodiments, at least one known amount of a biomarker is higher than a corresponding amount in the sample or in a control.

In some embodiments, the biomarker comprises: one or more proteins selected from the group comprising phenylalanine-4-hydroxylase, glycine N-methyltransferase, glyoxylate/hydroxypyruvate reductase, 2′-5′-oligoadenylate synthetase, 4-hydroxyphenylpyruvate dioxygenase, and homogentisate 1,2-dioxygenase; one or more dipeptides selected from the group comprising arginine-glutamine, arginine-alanine, arginine valine, and lysine-glutamine; or a combination of any of the foregoing.

In some embodiments, detecting the environmental stress or an effect thereof comprises: measuring a first amount of a biomarker in a first sample from the marine invertebrate; measuring a second amount of the biomarker in a control or in a second sample from the marine invertebrate; and comparing the first and second amounts of the biomarker, wherein the second amount relative to the first amount indicates that the marine invertebrate is likely or predicted to be affected by environmental stress.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a principal coordinate analysis (PCoA) plot and shows the relationship between proteomic samples from one genotype (MC-289) of Montipora capitata (M. capitata).

FIG. 1B is a PCoA plot and shows the relationship between transcriptomic samples from one genotype (MC-289) of M. capitata. FIG. 1C is a PCoA plot and shows the relationship between transcripts with proteomic evidence from samples from one genotype (MC-289) of M. capitata.

FIG. 1D is a sparse partial least squares-discriminant analysis (sPLS-DA) plot and shows the relationship between proteomic samples from one genotype (MC-289) of M. capitata. FIG. 1E is a sPLS-DA plot and shows the relationship between transcriptomic samples from one genotype (MC-289) of M. capitata. FIG. 1F is a sPLS-DA plot and shows the relationship between transcripts with proteomic evidence from samples from one genotype (MC-289) of M. capitata.

FIG. 1G is a principal component analysis (PCA) plot of proteomic samples from one genotype (MC-289) of M. capitata. FIG. 1H shows a PCA plot of transcriptomic samples from one genotype (MC-289) of M. capitata. FIG. 1I shows a PCA plot of transcripts with proteomic evidence from samples from one genotype (MC-289) of M. capitata. For FIGS. 1A-1I, the shading of each data point corresponds to the treatment (white, ambient (Amb); gray, high temperature (HiT); or black, field samples) and the symbol shape corresponds to the treatment and time point at which each sample was collected. Samples from the same condition (treatment and time point) are grouped with ellipses. The amount (%) of variance explained by each axis in each plot is displayed in parentheses. Samples derived from mislabeled genotypes (i.e., samples that are not MC-289) are annotated with their respective plug IDs (2998 for MC-289_T5-HIT_2998 and 1721 for MC-289_T5-Amb_1721).

FIG. 2 shows the proportion (%) of single nucleotide polymorphisms (SNPs) shared between each of the MC-289 transcriptome samples. The heatmap shows the proportion of shared SNPs between all pairwise combinations of the MC-289 transcriptome samples. The value of each pairwise combination is shown on the heatmap; the shade of each cell corresponds with the color key and reflects the percentage of shared SNPs. A histogram showing the number of pairwise comparisons (count) with a given proportion of shared SNPs (percent shared SNPs) is shown in the top left of the figure; the background shades used in the histogram correspond to the shades used in the heatmap. A legend describing the patterns used along the top and left sides of the heatmap and corresponding treatments and time points is presented at the top right of the figure. Sample numbers are shown along the lower and right sides of the heatmap. The order of the columns and rows, and the dendrograms presented on the top and left sides of the heatmap, were generated by hierarchical clustering of the proportion of shared SNPs between each of the samples. The values presented in the heatmap are reproduced in Table 2 in the Exemplification.

FIG. 3A shows the log₂ (fold-change) (log₂FC) of transcript (x-axis) and protein (y-axis) expression values between high vs. ambient temperature samples for time point 1 (TP1).

FIG. 3B shows the log₂FC of transcript (x-axis) and protein (y-axis) expression values between high vs. ambient temperature samples for time point 3 (TP3). FIG. 3C shows the log₂FC of transcript (x-axis) and protein (y-axis) expression values between high vs. ambient temperature samples for time point 5 (TP5). For FIGS. 3A-3C, each plot is divided into four quadrants (Q1-Q4): Q1 contains genes with positive protein and transcript log₂FC values, Q2 contains genes with positive protein and negative transcript log₂FC values, Q3 contains genes with negative proteins and transcript log₂FC values, and Q4 contains genes with negative protein and positive transcript log₂FC values. A trend line (bold) is fitted through the data with associated R² value and line formula shown in the top left corner. Data points represent differentially expressed genes/transcripts (DEGs, white), differentially expressed proteins (DEPs, light gray), instances where both a gene and its protein product were differentially expressed (DEG and DEP, black), and genes/proteins not differentially expressed (dark gray).

FIGS. 4A-4N show correlation between the log₂ transformed transcript expression (log₂ (transcripts per kilobase million+1); transcripts per kilobase million is abbreviated as TPM) and protein expression (log₂ (normalized abundance+1)) values of the genes detected in the proteomic data (n=4036) from each treatment group. A trend line (bold white) is fitted through the data with associated R² value and line formula shown in the upper left corner of each plot. FIG. 4A shows data from sample MC-289_T1-Amb_1603. FIG. 4B shows data from sample MC-289_T1-Amb_1609. FIG. 4C shows data from sample MC-289_T1-HIT_2023. FIG. 4D shows data from sample MC-289_T1-HIT_2878. FIG. 4E shows data from sample MC-289_T3-Amb_1595. FIG. 4F shows data from sample MC-289_T3-Amb_2741. FIG. 4G shows data from sample MC-289_T3-HiT_2058. FIG. 4H shows data from sample MC-289_T3-HIT_2183. FIG. 4I shows data from sample MC-289_T5-Amb_1721. FIG. 4J shows data from sample MC-289_T5-Amb_2874. FIG. 4K shows data from sample MC-289_T5-HIT_1341.

FIG. 4L shows data from sample MC-289_T5-HIT_2998. FIG. 4M shows data from sample MC-289_Field_289-1. FIG. 4N shows data from sample MC-289_Field_289-2.

FIG. 5 shows average color scores of M. capitata coral nubbins under ambient (bold line) and high (thin line) temperature treatment conditions over the course of the experiment. Error bars are shown for the color scores observed at each time point. The time points (T1, T3, and T5) when samples were collected for-omics data generation are labeled.

FIG. 6A is a PCoA plot and shows the relationship between metabolomic samples from one genotype (MC-289) of M. capitata. FIG. 6B is a sPLS-DA plot and shows the relationship between metabolomic samples from one genotype (MC-289) of M. capitata. FIG. 6C is a PCA plot and shows the relationship between metabolomic samples from one genotype (MC-289) of M. capitata. FIG. 6D is a sPLS-DA plot and shows the relationship between metabolomic samples from four genotypes of M. capitata. FIG. 6E is a PCA plot and shows the relationship between metabolomic samples from four genotypes of M. capitata. FIG. 6F is a PCoA plot and shows the relationship between metabolomic samples from four genotypes of M. capitata. For FIGS. 6A-6C, the shape of each point corresponds to the treatment (i.e., ambient (Amb), high temperature (HiT), or field) and time point (T1, T3, or T5) at which each sample was collected; the shade corresponds to the treatment (white, Ambient; black, Field; gray, High Temperature). For FIGS. 6D-6F, the shape of each point corresponds to the treatment (i.e., ambient (Amb), high temperature (HiT), or field) and time point (T1, T3, or T5) at which each sample was collected; the shade or pattern of each point corresponds to the genotype (white, 206; black, 248; gray, 289; hatched, 291). Samples from the same condition are grouped with ellipses. The amount of variance explained by each axis in each plot is displayed in parentheses (%). PCoA plots are based on Bray-Curtis distances between all samples in the corresponding dataset. Samples derived from mislabeled genotypes (i.e., samples that are not MC-289) are annotated with their respective plug IDs (2998 for MC-289_T5-HIT_2998 and 1721 for MC-289_T5-Amb_1721).

FIG. 7A shows the relationship between 16S microbiome samples from one genotype (MC-289) of M. capitata presented as a PCoA plot based on Bray-Curtis distances. FIG. 7B shows the relationship between 16S microbiome samples from one genotype (MC-289) of M. capitata presented as a PLS-DA (sPLS-DA) plot. FIG. 7C shows the relationship between 16S microbiome samples from one genotype (MC-289) of M. capitata presented as a PCA plot. FIG. 7D shows the relationship between microbiome samples from four genotypes of M. capitata presented as PLS-DA (sPLS-DA) plot. FIG. 7E shows the relationship between microbiome samples from four genotypes of M. capitata presented as PCA plot. FIG. 7F shows the relationship between microbiome samples from four genotypes of M. capitata presented as a PCoA plot. For FIGS. 7A-7C, the shape of each point corresponds to the treatment (i.e., ambient (Amb), high temperature (HiT), or field) and time point (T1, T3, or T5) at which each sample was collected; the shade corresponds to the treatment (white, Ambient; black, Field; gray, High Temperature); a legend with this information is displayed on right of the plot. For FIGS. 7D-7F, the shape of each point corresponds to the treatment (i.e., ambient (Amb), high temperature (HiT), or field) and time point (T1, T3, or T5) at which each sample was collected; the shade or pattern of each point corresponds to the genotype (white, 206; black, 248; gray, 289; hatched, 291). Samples from the same condition are grouped with ellipses. The amount of variance explained by each axis in each plot is displayed in parentheses (%). PCoA plots are based on the Bray-Curtis distances between all samples in the corresponding dataset. Samples derived from mislabeled genotypes (i.e., samples that are not MC-289) are annotated with their respective plug IDs (2998 for MC-289_T5-HiT_2998 and 1721 for MC-289_T5-Amb_1721). FIG. 7G shows change in 16S microbiome sample α-diversity over time for each genotype. Boxplots show the spread of sample Simpsons α-diversity values for each of the four genotypes over the sampled time points. The values presented along the y-axis are the inverse α-diversity measure values (i.e., larger values [closer to one] represent samples with higher diversity and smaller values [closer to zero] represent samples with lower diversity).

FIG. 8A depicts a spectral comparison for three coral stress metabolites (ornithine, top panel; cytidine-5′-diphosphocholine (CDP-choline), middle panel; and phosphocholine, lower panel) via mirror plots. The signal derived from the sample is shown in a thin trace on top and the signal derived from the chemical standard is shown in a bold trace on the bottom of each plot. FIGS. 8B-8E show dipeptide metabolite concentrations in the pooled sample from M. capitata collected under the high temperature ambient partial pressure of CO₂ (pCO₂) (HTAC; i.e., thermal stress) treatment. Signals of the endogenous dipeptide levels (thin line) were compared to those of a mix of stable isotope labeled dipeptide internal standards (STDs or IS; bold line). The points of intersection of the traces identify the concentrations of the endogenous dipeptide levels in the samples. FIG. 8B shows the concentration of arginine-glutamine (RQ) in a sample compared to labeled standard (13C6-15N4 arginine-glutamine). FIG. 8C shows the mass spectrometry intensity values of arginine-alanine (RA) in a sample compared to that of a labeled standard with a known concentration (13C6-15N4 arginine-alanine). FIG. 8D shows the mass spectrometry intensity values of arginine-valine (RV) in a sample compared to that of a labeled standard with a known concentration (13C6-15N4 arginine-valine). FIG. 8E shows mass spectrometry intensity values of lysine-glutamine (KQ) in a sample compared to that of a labeled standard with a known concentration (13C6-15N4 lysine-glutamine).

FIGS. 9A-9D show dipeptide metabolite ion counts in pooled samples from M. capitata (MCap) and Pocillopora acuta (P. acuta, PAcu) collected under thermal stress (HT) treatment or ambient temperature (AT) controls. Signals of the endogenous dipeptide levels (white bars) were compared to those of stable isotope labeled dipeptide internal standards (black bars). FIG. 9A shows the ion count of arginine-glutamine (RQ) in samples compared to labeled standard (500 nM 13C6-15N4 arginine-glutamine). FIG. 9B shows the ion count of arginine-alanine (RA) in samples compared to labeled standard (100 nM 13C6-15N4 arginine-alanine).

FIG. 9C shows the ion count of arginine-valine (RV) in samples compared to labeled standard (10 nM 13C6-15N4 arginine-valine). FIG. 9D shows the ion count of lysine-glutamine (KQ) in samples compared to labeled standard (100 nM 13C6-15N4 lysine-glutamine).

FIGS. 10A-10D show dipeptide metabolite normalized ion counts in pooled samples from M. capitata (MCap) and P. acuta (PAcu) collected under thermal stress (HT) treatment or ambient temperature (AT) controls. Signals of the endogenous dipeptide levels were normalized to those of stable isotope labeled dipeptide internal standards (shown in FIGS. 9A-9D). FIG. 10A shows the normalized ion count of arginine-glutamine (RQ) in samples. FIG. 10B shows the normalized ion count of arginine-alanine (RA) in samples. FIG. 10C shows the normalized ion count of arginine-valine (RV) in samples. FIG. 10D shows the normalized ion count of lysine-glutamine (KQ) in samples.

FIGS. 11A-11D show dipeptide metabolite absolute amounts (pmol) in pooled samples from M. capitata (MCap) and P. acuta (PAcu) collected under thermal stress (HT) treatment or ambient temperature (AT) controls. FIG. 11A shows the absolute amount of arginine-glutamine (RQ) in samples. FIG. 11B shows the absolute amount of arginine-alanine (RA) in samples. FIG. 11C shows the absolute amount of arginine-valine (RV) in samples. FIG. 11D shows the absolute amount of lysine-glutamine (KQ) in samples.

FIGS. 12A-12D show weight-normalized dipeptide metabolite concentrations (pmol/g) in pooled samples from M. capitata (MCap) and P. acuta (PAcu) collected under thermal stress (HT) treatment or ambient temperature (AT) controls. FIG. 12A shows the concentration of arginine-glutamine (RQ) in samples. FIG. 12B shows the concentration of arginine-alanine (RA) in samples. FIG. 12C shows the concentration of arginine-valine (RV) in samples. FIG. 12D shows the concentration of lysine-glutamine (KQ) in samples.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, model organisms, and animals. The present disclosure is not limited by the illustrated ordering of acts or events, as some acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

### Definitions

### General

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

It should be understood that for all numerical bounds describing some parameter in this application, such as “about,” “at least,” “less than,” “fewer than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

As used herein, the term “about” means within an acceptable error range for a particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of +20%, e.g., +10%, +5% or +1% of a given value. It is to be understood that the term “about” can precede any particular value specified herein, except for particular values used in the Exemplification. When “about” precedes a range, as in “1-20”, the term “about” should be read as applying to both given values of the range, such that “about 1-20” means about 1 to about 20.

### Subject Matter

As used herein, the term “polypeptide” or “protein” refers to a polymer of at least two amino acids covalently linked by an amide bond, regardless of length or post-translational modification (e.g., glycosylation or phosphorylation). A polypeptide can comprise a naturally occurring protein. A polypeptide can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitrulline, homoserine, norleucine, norvaline, ornithine). The amino, carboxyl, and/or other functional groups on a polypeptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a polypeptide can also be derivatized (e.g., alkylated) or labeled (e.g., with a detectable label, such as a fluorogen or a hapten) using methods known in the art. A polypeptide can comprise one or more modifications (e.g., amino acid linkers, acylation, acetylation, amidation, methylation, terminal modifiers (e.g., cyclizing modifications), N-methyl-a-amino group substitution), if desired. In addition, a polypeptide can be an analog of a known and/or naturally occurring peptide, for example, a peptide analog having conservative amino acid residue substitution(s).

As used herein, the term “antibody” refers to an immunoglobulin molecule capable of specific binding to a target, such as a carbohydrate, polynucleotide, lipid, polypeptide, etc., through at least one antigen recognition site, located in the variable domain of the immunoglobulin molecule. As used herein, the term “antibody” refers to a full-length antibody. In some embodiments, an antibody is a modified and/or engineered antibody; non-limiting examples of modified and/or engineered antibodies include chimeric antibodies, humanized antibodies, multiparatopic antibodies, bispecific antibodies, and multispecific antibodies.

As used herein, the term “antibody mimetic” refers to polypeptides capable of mimicking an antibody's ability to bind an antigen, but structurally differ from native antibody structures. Examples of an antibody mimetic include, but are not limited to, Adnectin, AFFIBODY®, Affilin, Affimer, Affitin, Alphabody, ANTICALIN®, Avimer, DARPIN®, Fynomer, Kunitz domain peptide, monobody, nanoCLAMP, and Versabody.

As used herein, the term “antigen-binding fragment” refers to a portion of an immunoglobulin molecule (e.g., antibody) that retains the antigen binding properties (e.g., of a corresponding full-length antibody). Non-limiting examples of antigen-binding fragments include a heavy chain variable (V_{H}) region, a single-domain antibody (sdAb), a light chain variable (V_{L}) region, a fragment antigen-binding region (Fab fragment), a divalent antibody fragment (F(ab′)₂ fragment), a Fd fragment, a Fv fragment, and a domain antibody (dAb) consisting of one V_{H} domain or one V_{L} domain, etc. V_{H} and VI, domains may be linked together via a synthetic linker to form various types of single-chain antibody designs in which the V_{H}/V_{L} domains pair intramolecularly, or intermolecularly in those cases when the V_{H} and V_{L} domains are expressed by separate chains, to form a monovalent antigen binding site, such as single chain Fv (scFv) or diabody. In some embodiments, an antigen-binding fragment is Fab, F(ab′)₂, Fab′, scFv, or Fv. In some embodiments, antigen-binding fragment is a scFv.

“Treating” or “treatment,” as used herein, refers to taking steps to benefit the health of a subject, such as a marine invertebrate, in need thereof (e.g., as by implementing an intervention). “Treating” or “treatment” includes inhibiting the disease or condition (e.g., as by slowing or stopping its progression or causing regression of the disease or condition) and relieving the symptoms (e.g., bleaching) resulting from the disease or condition.

The term “treating” or “treatment” refers to the management of a subject (e.g., coral) with the intent to improve, ameliorate, stabilize (i.e., not worsen), prevent, or cure a disease or pathological condition—such as the particular indications of environmental stress exemplified herein. This term includes active treatment (treatment directed to improve the disease or pathological condition), causal treatment (treatment directed to the cause of the associated disease or pathological condition), preventative treatment (treatment directed to minimizing or partially or completely inhibiting the development of the associated disease or pathological condition), and supportive treatment (treatment employed to supplement another therapy). Treatment also includes diminishment of the extent of the disease or condition, preventing spread of the disease or condition, delay or slowing the progress of the disease or condition, amelioration or palliation of the disease or condition, and remission (whether partial or total), whether detectable or undetectable. “Ameliorating” or “palliating” a disease or condition means that the extent and/or undesirable manifestations of the disease or condition are lessened and/or time course of the progression is slowed or lengthened, as compared to the extent or time course in the absence of treatment. “Treatment” can also mean prolonging survival as compared to expected survival if not receiving treatment. Subjects (e.g., coral) in need of treatment include those already with the condition or disease, as well as those prone to have the condition or disease or those in which the condition or disease is to be prevented. Desired response or desired results in response to “treating” or “treatment” include effects at the cellular level, tissue level, organismal level, population level, holobiont level, or a combination thereof.

As used herein, the term “reference” may refer to a standard or control condition (e.g., untreated with a test agent or combination of test agents). Alternatively, “reference” may refer to a resource, such as an annotated genome, transcriptome, or the like, that is used to assemble, analyze, and/or interpret data.

### Embodiments

The present disclosure provides non-limiting examples of embodiments of:
a device for detecting environmental stress or an effect thereof in a sample from a marine invertebrate, wherein the device detects a biomarker;
use of a device for detecting environmental stress or an effect thereof in a sample from a marine invertebrate for: monitoring health of a marine invertebrate; detecting environmental stress or an effect thereof in a marine invertebrate; detecting viability, variation, abundance, proliferation, or fecundity of a marine invertebrate; treating a marine invertebrate; identifying a marine invertebrate for propagation or protection; or the like; or a combination of any of the foregoing;
a composition for use as a standard in detecting environmental stress or an effect thereof in a sample from a marine invertebrate, wherein the composition comprises one or more known amounts of one or more biomarkers;
a kit comprising: a device for detecting environmental stress or an effect thereof in a sample from a marine invertebrate, wherein the device detects a biomarker; or a composition for use as a standard in detecting environmental stress or an effect thereof in a sample from a marine invertebrate, wherein the composition comprises one or more known amounts of one or more biomarkers; and
a method for detecting environmental stress or an effect thereof in a sample from a marine invertebrate.

### Environmental Stress and Effects Thereof

The following embodiments describe non-limiting examples of environmental stress and/or an effect thereof in relation to:
a device for detecting environmental stress or an effect thereof in a sample from a marine invertebrate, wherein the device detects a biomarker;
use of a device for detecting environmental stress or an effect thereof in a sample from a marine invertebrate for: monitoring health of a marine invertebrate; detecting environmental stress or an effect thereof in a marine invertebrate; detecting viability, variation, abundance, proliferation, or fecundity of a marine invertebrate; treating a marine invertebrate; identifying a marine invertebrate for propagation or protection; or the like; or a combination of any of the foregoing;
a composition for use as a standard in detecting environmental stress or an effect thereof in a sample from a marine invertebrate, wherein the composition comprises one or more known amounts of one or more biomarkers;
a kit comprising: a device for detecting environmental stress or an effect thereof in a sample from a marine invertebrate, wherein the device detects a biomarker; or a composition for use as a standard in detecting environmental stress or an effect thereof in a sample from a marine invertebrate, wherein the composition comprises one or more known amounts of one or more biomarkers; and
a method for detecting environmental stress or an effect thereof in a sample from a marine invertebrate.

In some embodiments, environmental stress comprises, involves, or is related to heat stress, thermal stress, temperature sensitivity-inducing stress, stress as a function of temperature, and the like. In some embodiments, environmental stress comprises thermal stress. In some embodiments, environmental stress comprises heat stress. In some embodiments, environmental stress comprises temperature sensitivity-inducing stress. In some embodiments, environmental stress comprises stress as a function of temperature. In some embodiments, environmental stress comprises more than one type of environmental stress.

In some embodiments, an effect of environmental stress comprises at least one effect selected from the group comprising: reduced health, increased stress, reduced viability, reduced variation, reduced abundance, reduced proliferation, and reduced fecundity. In some embodiments, an effect of environmental stress comprises reduced health, e.g., a reduction in health of a marine invertebrate or population of marine invertebrates. In some embodiments, an effect of environmental stress comprises increased stress, e.g., increased overall stress in a marine invertebrate or population of marine invertebrates. In some embodiments, an effect of environmental stress comprises reduced viability, e.g., a reduction in a proportion of living to deceased marine invertebrates in a population. In some embodiments, an effect of environmental stress comprises reduced variation, e.g., a reduction in species diversity among a heterogeneous marine invertebrate population. In some embodiments, an effect of environmental stress comprises reduced abundance, e.g., a reduction in number of living marine invertebrates in a population. In some embodiments, an effect of environmental stress comprises reduced proliferation, e.g., a reduction in a rate at which an individual marine invertebrate or a population of marine invertebrates reproduces. In some embodiments, an effect of environmental stress comprises reduced fecundity, e.g., a reduction in a reproductive potential of a marine invertebrate or population of marine invertebrates. In some embodiments, an effect of environmental stress comprises bleaching of a marine invertebrate or population of marine invertebrates. In some embodiments, an effect of environmental stress comprises more than one effect.

### Marine Invertebrates

The following embodiments describe non-limiting examples of a marine invertebrate in relation to:
a device for detecting environmental stress or an effect thereof in a sample from a marine invertebrate, wherein the device detects a biomarker;
use of a device for detecting environmental stress or an effect thereof in a sample from a marine invertebrate for: monitoring health of a marine invertebrate; detecting environmental stress or an effect thereof in a marine invertebrate; detecting viability, variation, abundance, proliferation, or fecundity of a marine invertebrate; treating a marine invertebrate; identifying a marine invertebrate for propagation or protection; or the like; or a combination of any of the foregoing;
a composition for use as a standard in detecting environmental stress or an effect thereof in a sample from a marine invertebrate, wherein the composition comprises one or more known amounts of one or more biomarkers;
a kit comprising: a device for detecting environmental stress or an effect thereof in a sample from a marine invertebrate, wherein the device detects a biomarker; or a composition for use as a standard in detecting environmental stress or an effect thereof in a sample from a marine invertebrate, wherein the composition comprises one or more known amounts of one or more biomarkers; and
a method for detecting environmental stress or an effect thereof in a sample from a marine invertebrate.

In some embodiments, a marine invertebrate comprises a holobiont. As used herein, the term “holobiont” refers to a host species and symbiotic species associated therewith. In some embodiments, the holobiont comprises one or more cnidarian animal hosts, one or more algal symbionts, and one or more organisms belonging to one or more other taxa. In some embodiments, the holobiont comprises a diverse population of algal symbionts, e.g., Symbiodiniaceae. In some embodiments, the holobiont comprises one or more organisms belonging to one or more diverse taxa, e.g., fungi, protists, prokaryotes, and/or viruses. In some embodiments, a marine invertebrate comprises coral or coral nubbins. In some embodiments, a marine invertebrate comprises Montipora capitata (M. capitata) or Pocillopora acuta (P. acuta). In some embodiments, a marine invertebrate comprises a shellfish. In some embodiments, a shellfish comprises a mollusk, a crustacean, or an echinoderm.

In some embodiments, a marine invertebrate comprises one marine invertebrate. In some embodiments, a marine invertebrate comprises two or more marine invertebrates. In some embodiments, a marine invertebrate comprises an individual organism or host organism and its symbionts. In some embodiments, a marine invertebrate comprises a population of organisms and/or host organisms and their symbionts. In some embodiments, a marine invertebrate comprises a homogenous population of organisms and/or host organisms and their symbionts. In some embodiments, a marine invertebrate comprises a heterogeneous population of organisms and/or host organisms and their symbionts.

### Samples

As used herein, the term “sample” refers to a portion of a marine invertebrate. In some embodiments, a sample comprises a portion of an individual organism or host organism and its symbionts. In some embodiments, a sample comprises a portion of a population of marine invertebrates. In some embodiments, a sample comprises a portion of a population of organisms and/or host organisms and their symbionts. In some embodiments, a sample comprises a one or more individual organisms and/or host organisms and their symbionts as a representation of a population of organisms and/or host organisms and their symbionts. In some embodiments, a sample comprises a portion (e.g., a piece or a small piece) of coral. In some embodiments, a sample comprises a coral nubbin. In some embodiments, a sample comprises a mixture of samples (i.e., a pooled sample) from: two or more portions of an individual organism or host organism and its symbionts, two or more individual organisms or host organisms and their symbionts, or both.

In some embodiments, a sample comprises a homogenate derived from a marine invertebrate or a portion thereof. In some embodiments, a sample comprises a concentrate, a lyophilized material, a frozen material, a preserved material, a desiccated material, or the like derived from a marine invertebrate or a portion thereof. In some embodiments, a sample comprises nucleic acids (e.g., DNA or RNA), proteins, metabolites (e.g., dipeptides), microbiota, or the like isolated from a marine invertebrate or a portion thereof. As used herein, the term “isolated” refers to a material that is free to varying degrees from components which normally accompany it as found in its original or natural state. “Isolate” denotes a degree of separation from original source or surroundings. In some embodiments, an isolated material is considered to be substantially free of other components. In some embodiments, a sample comprising nucleic acids (e.g., DNA or RNA), proteins, metabolites (e.g., dipeptides), microbiota, or the like is substantially pure from contaminating substances. In some embodiments, a sample comprises DNA isolated from a marine invertebrate or a portion thereof. In some embodiments, a sample comprises RNA isolated from a marine invertebrate or a portion thereof. In some embodiments, a sample comprises protein isolated from a marine invertebrate or a portion thereof. In some embodiments, a sample comprises metabolites isolated from a marine invertebrate or a portion thereof. In some embodiments, a sample comprises microbiota isolated from a marine invertebrate or a portion thereof.

### Biomarkers

The following embodiments describe non-limiting examples of a biomarker in relation to:
a device for detecting environmental stress or an effect thereof in a sample from a marine invertebrate, wherein the device detects a biomarker;
use of a device for detecting environmental stress or an effect thereof in a sample from a marine invertebrate for: monitoring health of a marine invertebrate; detecting environmental stress or an effect thereof in a marine invertebrate; detecting viability, variation, abundance, proliferation, or fecundity of a marine invertebrate; treating a marine invertebrate; identifying a marine invertebrate for propagation or protection; or the like; or a combination of any of the foregoing;
a composition for use as a standard in detecting environmental stress or an effect thereof in a sample from a marine invertebrate, wherein the composition comprises one or more known amounts of one or more biomarkers;
a kit comprising: a device for detecting environmental stress or an effect thereof in a sample from a marine invertebrate, wherein the device detects a biomarker; or a composition for use as a standard in detecting environmental stress or an effect thereof in a sample from a marine invertebrate, wherein the composition comprises one or more known amounts of one or more biomarkers; and
a method for detecting environmental stress or an effect thereof in a sample from a marine invertebrate.

As used herein, the term “biomarker” refers to a biological (or biologically derived or biologically associated) detectable quality, characteristic, substance, or the like that reflects, indicates, or is associated with a biological process, event, condition, or the like. In some embodiments, a detectable quality, characteristic, substance, or the like is qualitatively detectable. In some embodiments, a detectable quality, characteristic, substance, or the like is quantitatively detectable. In some embodiments, a biological process, event, condition, or the like comprises environmental stress.

In some embodiments, a biomarker, i.e., of environmental stress in a marine invertebrate, is selected from the group comprising: phenylalanine-4-hydroxylase (PAH), glycine N-methyltransferase (GNMT), glyoxylate/hydroxypyruvate reductase (GHR), 2′-5′-oligoadenylate synthetase (OAS1_C), 4-hydroxyphenylpyruvate dioxygenase (HPPD), and homogentisate 1,2-dioxygenase (HGD). In some embodiments, an antibody binds a biomarker. In some embodiments, an antibody binds one or more biomarkers. In some embodiments, an antibody binds phenylalanine-4-hydroxylase, glycine N-methyltransferase, glyoxylate/hydroxypyruvate reductase, 2′-5′-oligoadenylate synthetase, 4-hydroxyphenylpyruvate dioxygenase, or homogentisate 1,2-dioxygenase. In some embodiments, an antibody binds phenylalanine-4-hydroxylase. In some embodiments, an antibody binds glycine N-methyltransferase. In some embodiments, an antibody binds glyoxylate/hydroxypyruvate reductase. In some embodiments, an antibody binds 2′-5′-oligoadenylate synthetase. In some embodiments, an antibody binds 4-hydroxyphenylpyruvate dioxygenase. In some embodiments, an antibody binds homogentisate 1,2-dioxygenase.

In some embodiments, a biomarker comprises a protein, a dipeptide, a metabolite, a transcript (i.e., RNA), an aspect of a microbiome (e.g., 16S-ribosomal RNA), or the like. In some embodiments, a metabolite comprises a dipeptide. In some embodiments, a biomarker comprises more than one protein, dipeptide, metabolite, transcript (i.e., RNA), aspect of a microbiome (e.g., 16S-ribosomal RNA), or the like.

In some embodiments, a biomarker, i.e., a protein, comprises phenylalanine-4-hydroxylase. In some embodiments, a biomarker, i.e., a protein, comprises glycine N-methyltransferase. In some embodiments, a biomarker, i.e., a protein, comprises glyoxylate/hydroxypyruvate reductase. In some embodiments, a biomarker, i.e., a protein, comprises 2′-5′-oligoadenylate synthetase. In some embodiments, a biomarker, i.e., a protein, comprises 4-hydroxyphenylpyruvate dioxygenase. In some embodiments, a biomarker, i.e., a protein, comprises homogentisate 1,2-dioxygenase.

In some embodiments, a biomarker, i.e., a dipeptide, comprises arginine-glutamine (RQ). In some embodiments, a biomarker, i.e., a dipeptide, comprises arginine-alanine (RA). In some embodiments, a biomarker, i.e., a dipeptide, comprises arginine-valine (RV). In some embodiments, a biomarker, i.e., a dipeptide, comprises lysine-glutamine (KQ).

In some embodiments, a biomarker, i.e., a metabolite, comprises ornithine. In some embodiments, a biomarker, i.e., a metabolite, comprises cytidine-5′-diphosphocholine (CDP-choline). In some embodiments, a biomarker, i.e., a metabolite, comprises phosphocholine.

In some embodiments, a biomarker, i.e., a transcript, is related to glycolysis or gluconcogenesis. In some embodiments, a biomarker, i.e., a transcript, is related to the citrate cycle (tricarboxylic acid cycle or TCA cycle). In some embodiments, a biomarker, i.e., a transcript, is related to the pentose phosphate pathway. In some embodiments, a biomarker, i.e., a transcript, is related to amino sugar and nucleotide sugar metabolism. In some embodiments, a biomarker, i.e., a transcript, is related to pyruvate metabolism. In some embodiments, a biomarker, i.e., a transcript, is related to oxidative phosphorylation. In some embodiments, a biomarker, i.e., a transcript, is related to nitrogen metabolism. In some embodiments, a biomarker, i.e., a transcript, is related to fatty acid biosynthesis. In some embodiments, a biomarker, i.e., a transcript, is related to steroid hormone biosynthesis. In some embodiments, a biomarker, i.e., a transcript, is related to glycerolipid metabolism. In some embodiments, a biomarker, i.e., a transcript, is related to glycerophospholipid metabolism. In some embodiments, a biomarker, i.e., a transcript, is related to purine metabolism. In some embodiments, a biomarker, i.e., a transcript, is related to pyrimidine metabolism. In some embodiments, a biomarker, i.e., a transcript, is related to alanine, aspartate, and glutamate metabolism. In some embodiments, a biomarker, i.e., a transcript, is related to glycine, serine, and threonine metabolism. In some embodiments, a biomarker, i.e., a transcript, is related to cysteine and methionine metabolism. In some embodiments, a biomarker, i.e., a transcript, is related to valine, leucine, and isoleucine degradation. In some embodiments, a biomarker, i.e., a transcript, is related to valine, leucine, and isoleucine biosynthesis. In some embodiments, a biomarker, i.e., a transcript, is related to lysine biosynthesis. In some embodiments, a biomarker, i.e., a transcript, is related to lysine degradation. In some embodiments, a biomarker, i.e., a transcript, is related to arginine biosynthesis. In some embodiments, a biomarker, i.e., a transcript, is related to arginine and proline metabolism. In some embodiments, a biomarker, i.e., a transcript, is related to histidine metabolism. In some embodiments, a biomarker, i.e., a transcript, is related to tyrosine metabolism. In some embodiments, a biomarker, i.e., a transcript, is related to phenylalanine metabolism. In some embodiments, a biomarker, i.e., a transcript, is related to tryptophan metabolism. In some embodiments, a biomarker, i.e., a transcript, is related to phenylalanine, tyrosine, and tryptophan biosynthesis.

### Device for Detecting Environmental Stress

In one aspect, the present disclosure provides a device for detecting environmental stress or an effect thereof in a sample from a marine invertebrate, wherein the device detects a biomarker.

In some embodiments, a device comprises a test strip or a lateral flow immunoassay (LFIA). In some embodiments, a device comprises a test strip. In some embodiments, a device comprises a LFIA. In some embodiments, a test strip or LFIA comprises a substrate and a sensing chemistry.

In some embodiments, a sensing chemistry comprises an antibody. In some embodiments, an antibody comprises an antibody, an antibody mimetic, an antigen-binding fragment, or the like. In some embodiments, an antibody binds a biomarker. In some embodiments, a biomarker is selected from the group comprising: phenylalanine-4-hydroxylase, glycine N-methyltransferase, glyoxylate/hydroxypyruvate reductase, 2′-5′-oligoadenylate synthetase, 4-hydroxyphenylpyruvate dioxygenase, and homogentisate 1,2-dioxygenase. In some embodiments, an antibody binds phenylalanine-4-hydroxylase. In some embodiments, an antibody binds glycine N-methyltransferase. In some embodiments, an antibody binds glyoxylate/hydroxypyruvate reductase. In some embodiments, an antibody binds 2′-5′-oligoadenylate synthetase. In some embodiments, an antibody binds 4-hydroxyphenylpyruvate dioxygenase. In some embodiments, an antibody binds homogentisate 1,2-dioxygenase.

In some embodiments, a test strip or LFIA provides a quantitative readout. In some embodiments, a test strip or LFIA provides a qualitative readout. In some embodiments, a test strip or LFIA provides a quantitative readout or a qualitative readout. In some embodiments, a test strip or LFIA provides a quantitative readout and a qualitative readout. In some embodiments, a test strip or LFIA is combined with one or more other devices.

In some embodiments, a biomarker is detected at a level about 2-fold higher or lower in the sample compared to a control. In some embodiments, a biomarker is detected at a level about 2-fold higher (i.e., about twice as much) in the sample compared to a control. In some embodiments, a biomarker is detected at a level about 2-fold lower (i.e., about half as much) in the sample compared to a control. In some embodiments, a biomarker is detected at a fold-change of about 2, 3, 4, 5, 6, 7, 8, 9, 10, or 11.

In some embodiments, a biomarker is detected at a level at least 2-fold higher or lower in the sample compared to a control. In some embodiments, a biomarker is detected at a level at least 2-fold higher (i.e., twice as much or more) in the sample compared to a control. In some embodiments, a biomarker is detected at a level at least 2-fold lower (i.e., half as much or less) in the sample compared to a control. In some embodiments, a biomarker is detected at a fold-change of at least 2 (i.e., a fold change ≥2 or a fold change ≤−2), 3, 4, 5, 6, 7, 8, 9, 10, or 11.

In some embodiments, a biomarker is detected in a sample of a marine invertebrate at a log₂ (fold-change) (log₂FC) of ≥1 or ≤−1 in the sample compared to a control. In some embodiments, a biomarker is detected in a sample of a marine invertebrate at a log₂FC of at least 1 compared to a control. In some embodiments, a biomarker is detected in a sample of a marine invertebrate at a log₂FC of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, or more compared to a control. In some embodiments, a biomarker is detected in a sample of a marine invertebrate at a log₂FC of about 1.29, 1.46, 1.50, 1.60, 2.33, or 3.40 compared to a control. In some embodiments, a biomarker is detected in a sample of a marine invertebrate at a log₂FC of about 1.29 compared to a control. In some embodiments, a biomarker is detected in a sample of a marine invertebrate at a log₂FC of about 1.46 compared to a control. In some embodiments, a biomarker is detected in a sample of a marine invertebrate at a log₂FC of about 1.50 compared to a control. In some embodiments, a biomarker is detected in a sample of a marine invertebrate at a log₂FC of about 1.60 compared to a control. In some embodiments, a biomarker is detected in a sample of a marine invertebrate at a log₂FC of about 2.33 compared to a control. In some embodiments, a biomarker is detected in a sample of a marine invertebrate at a log₂FC of about 3.40 compared to a control. In some embodiments, a biomarker is detected in a sample of a marine invertebrate at a log₂FC of about −1.0, −1.1, −1.2, −1.3, −1.4, −1.5, −1.6, −1.7, −1.8, −1.9, −2.0, −2.1, −2.2, −2.3, −2.4, −2.5, −2.6, −2.7, −2.8, −2.9, −3.0, −3.1, −3.2, −3.3, −3.4, −3.5, −3.6, −3.7, −3.8, −3.9, −4.0, or less (i.e., more negative) compared to a control.

In some embodiments, a biomarker is detected in a sample of a marine invertebrate at a log₂FC of at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, or more compared to a control. In some embodiments, a biomarker is detected in a sample of a marine invertebrate at a log₂FC of at least 1.29, 1.46, 1.50, 1.60, 2.33, or 3.40 compared to a control. In some embodiments, a biomarker is detected in a sample of a marine invertebrate at a log₂FC of at least 1.29 compared to a control. In some embodiments, a biomarker is detected in a sample of a marine invertebrate at a log₂FC of at least 1.46 compared to a control. In some embodiments, a biomarker is detected in a sample of a marine invertebrate at a log₂FC of at least 1.50 compared to a control. In some embodiments, a biomarker is detected in a sample of a marine invertebrate at a log₂FC of at least 1.60 compared to a control. In some embodiments, a biomarker is detected in a sample of a marine invertebrate at a log₂FC of at least 2.33 compared to a control. In some embodiments, a biomarker is detected in a sample of a marine invertebrate at a log₂FC of at least 3.40 compared to a control. In some embodiments, a biomarker is detected in a sample of a marine invertebrate at a log₂FC of at least −1.0, −1.1, −1.2, −1.3, −1.4, −1.5, −1.6, −1.7, −1.8, −1.9, −2.0, −2.1, −2.2, −2.3, −2.4, −2.5, −2.6, −2.7, −2.8, −2.9, −3.0, −3.1, −3.2, −3.3, −3.4, −3.5, −3.6, −3.7, −3.8, −3.9, −4.0, or less (i.e., more negative) compared to a control.

In some embodiments, phenylalanine-4-hydroxylase is detected in a sample of a marine invertebrate at a log₂FC of at least 3.40 compared to a control. In some embodiments, glycine N-methyltransferase is detected in a sample of a marine invertebrate at a log₂FC of at least 2.33 compared to a control. In some embodiments, glyoxylate/hydroxypyruvate reductase is detected in a sample of a marine invertebrate at a log₂FC of at least 1.60 compared to a control. In some embodiments, 2′-5′-oligoadenylate synthetase is detected in a sample of a marine invertebrate at a log₂FC of at least 1.50 compared to a control. In some embodiments, 4-hydroxyphenylpyruvate dioxygenase is detected in a sample of a marine invertebrate at a log₂FC of at least 1.46 compared to a control. In some embodiments, homogentisate 1,2-dioxygenase is detected in a sample of a marine invertebrate at a log₂FC of at least 1.29 compared to a control.

### Uses

In another aspect, the present disclosure provides for use of a device for detecting environmental stress or an effect thereof in a sample from a marine invertebrate for: monitoring health of a marine invertebrate; detecting environmental stress or an effect thereof in a marine invertebrate; detecting viability, variation, abundance, proliferation, or fecundity of a marine invertebrate; treating a marine invertebrate; identifying a marine invertebrate for propagation or protection; or the like; or a combination of any of the foregoing. In some embodiments, use of a device for detecting environmental stress or an effect thereof in a sample from a marine invertebrate is for monitoring health of a marine invertebrate. In some embodiments, use of a device for detecting environmental stress or an effect thereof in a sample from a marine invertebrate is for detecting environmental stress or an effect thereof in a marine invertebrate. In some embodiments, use of a device for detecting environmental stress or an effect thereof in a sample from a marine invertebrate is for detecting viability of a marine invertebrate. In some embodiments, use of a device for detecting environmental stress or an effect thereof in a sample from a marine invertebrate is for detecting variation of a marine invertebrate. In some embodiments, use of a device for detecting environmental stress or an effect thereof in a sample from a marine invertebrate is for detecting abundance of a marine invertebrate. In some embodiments, use of a device for detecting environmental stress or an effect thereof in a sample from a marine invertebrate is for detecting proliferation of a marine invertebrate. In some embodiments, use of a device for detecting environmental stress or an effect thereof in a sample from a marine invertebrate is for detecting fecundity of a marine invertebrate. In some embodiments, use of a device for detecting environmental stress or an effect thereof in a sample from a marine invertebrate is for treating a marine invertebrate. In some embodiments, use of a device for detecting environmental stress or an effect thereof in a sample from a marine invertebrate is for identifying a marine invertebrate for propagation or protection. In some embodiments, use of a device for detecting environmental stress or an effect thereof in a sample from a marine invertebrate is for more than one purpose or intervention.

In some embodiments, treating a marine invertebrate comprises at least one intervention selected from the group comprising: prohibition of fishing, prohibition of swimming, prohibition of boating, and the like. In some embodiments, treating a marine invertebrate comprises prohibition of fishing. In some embodiments, treating a marine invertebrate comprises prohibition of swimming. In some embodiments, treating a marine invertebrate comprises prohibition of boating. In some embodiments, treating a marine invertebrate comprises prohibition of more than one human activity. In some embodiments, treating a marine invertebrate comprises implementation of a human activity. In some embodiments, treating a marine invertebrate comprises implementation of more than one human activity.

### Composition for Use as a Standard

In yet another aspect, the present disclosure provides a composition for use as a standard in detecting environmental stress or an effect thereof in a sample from a marine invertebrate, wherein the composition comprises one or more known amounts of one or more biomarkers.

As used herein, the term “standard” refers to a composition that is useful in determining an amount otherwise unknown. In some embodiments, a standard comprises a known amount, such as an absolute mass or a concentration, that is useful in determining an amount otherwise unknown.

For example, in some embodiments, a composition comprises a known amount, such as a mass (e.g., dry mass) or concentration (e.g., mg/mL, pmol/g, weight concentration (% w/w), volume concentration (% v/v), mass concentration (% w/v)) of a biomarker. When diluted in a known volume and/or weight of solvent, a composition provides a known concentration of a biomarker against which to measure an otherwise unknown concentration of a biomarker in a sample.

In some embodiments, a composition comprises a known amount, such as a concentration (e.g., pmol/g), of a biomarker against which to measure an otherwise unknown concentration of a biomarker in a sample. In some embodiments, a composition comprises a known concentration of a biomarker. In some embodiments, a composition comprises a known concentration of each of two or more biomarkers. In some embodiments, a composition comprises a known amount (e.g., dry mass) of a biomarker, wherein the known amount is to be diluted in a known volume and/or weight of solvent, thereby providing a composition of known concentration. In some embodiments, a composition comprises known amounts (e.g., dry masses) of two or more biomarkers, wherein the known amounts are to be diluted in one or more known volumes and/or weights of solvent, thereby providing a composition of known concentration. In some embodiments, a composition provides a standard curve of values associated with two or more known concentrations of each of one or more biomarkers, wherewith an otherwise unknown concentration of the one or more biomarkers in a sample can be determined.

In some embodiments, a composition comprises a known amount, such as a mass (e.g., dry mass), of a biomarker against which to measure an otherwise unknown mass of a biomarker in a sample. In some embodiments, a composition comprises a known amount of dry mass of a biomarker. In some embodiments, a composition comprises a known amount of dry mass of each of two or more biomarkers.

In some embodiments, a composition comprises one biomarker. In some embodiments, a composition comprises two or more biomarkers. In some embodiments, a biomarker is a protein. In some embodiments, a biomarker is a dipeptide.

In some embodiments, environmental stress comprises thermal stress. In some embodiments, an effect of environmental stress is at least one effect selected from the group comprising: reduced health, increased stress, reduced viability, reduced variation, reduced abundance, reduced proliferation, and reduced fecundity.

In some embodiments, a marine invertebrate comprises a holobiont. In some embodiments, a marine invertebrate comprises coral or coral nubbins.

In some embodiments, at least one known amount of a biomarker is higher than a corresponding amount in the sample or in a control. In some embodiments, at least one known amount of a biomarker is at least ten times (10×) higher than a corresponding amount in the sample or in a control.

In some embodiments, a biomarker comprises: one or more proteins selected from the group comprising phenylalanine-4-hydroxylase, glycine N-methyltransferase, glyoxylate/hydroxypyruvate reductase, 2′-5′-oligoadenylate synthetase, 4-hydroxyphenylpyruvate dioxygenase, and homogentisate 1,2-dioxygenase; one or more dipeptides selected from the group comprising arginine-glutamine, arginine-alanine, arginine-valine, and lysine-glutamine; or a combination of any of the foregoing.

In some embodiments, detecting the environmental stress or an effect thereof comprises: measuring a first amount of a biomarker in a first sample from the marine invertebrate; measuring a second amount of a biomarker in a control or in a second sample from the marine invertebrate; and comparing the first and second amounts of a biomarker, wherein the second amount relative to the first amount indicates that the marine invertebrate is likely or predicted to be affected by environmental stress.

In some embodiments, the first sample and the second sample are from the same individual, colony, population, or holobiont of the marine invertebrate; the first sample and the second sample are from different individuals, colonies, populations, or holobionts of the marine invertebrate; or the control is a standard.

### Kits Comprising a Device or Composition

In yet another aspect, the present disclosure provides a kit comprising: a device for detecting environmental stress or an effect thereof in a sample from a marine invertebrate, wherein the device detects a biomarker; or a composition for use as a standard in detecting environmental stress or an effect thereof in a sample from a marine invertebrate, wherein the composition comprises one or more known amounts of one or more biomarkers.

In some embodiments, environmental stress comprises, involves, or is related to heat stress, thermal stress, temperature sensitivity-inducing stress, stress as a function of temperature, and the like. In some embodiments, environmental stress comprises thermal stress.

In some embodiments, an effect of environmental stress is at least one effect selected from the group comprising: reduced health, increased stress, reduced viability, reduced variation, reduced abundance, reduced proliferation, and reduced fecundity.

In some embodiments, a marine invertebrate comprises a holobiont. In some embodiments, the marine invertebrate comprises coral or coral nubbins.

In some embodiments, a device comprises a test strip or a lateral flow immunoassay (LFIA). In some embodiments, a device comprises a test strip. In some embodiments, a device comprises a LFIA.

In some embodiments, a biomarker is detected at a level at least 2-fold higher or lower in the sample compared to a control.

In some embodiments, a biomarker is selected from the group comprising: phenylalanine-4-hydroxylase, glycine N-methyltransferase, glyoxylate/hydroxypyruvate reductase, 2′-5′-oligoadenylate synthetase, 4-hydroxyphenylpyruvate dioxygenase, and homogentisate 1,2-dioxygenase.

### Method for Detecting Environmental Stress

In some embodiments, the present disclosure provides a method for detecting environmental stress or an effect thereof in a sample from a marine invertebrate. In some embodiments, the method comprises: measuring a first amount of a biomarker in a first sample from the marine invertebrate; measuring a second amount of the biomarker in a control or in a second sample from the marine invertebrate; and comparing the first and second amounts of the biomarker, wherein the second amount relative to the first amount indicates that the marine invertebrate is likely or predicted to be affected by environmental stress.

In some embodiments, the first sample and the second sample are from the same individual, colony, population, or holobiont of the marine invertebrate; the first sample and the second sample are from different individuals, colonies, populations, or holobionts of the marine invertebrate; or the control is a standard, e.g., a composition for use as a standard in detecting environmental stress or an effect thereof in a sample from a marine invertebrate, wherein the composition comprises one or more known amounts of one or more biomarkers.

In some embodiments, a first amount and/or a second amount is an absolute amount. In some embodiments, a first amount and/or a second amount is an absolute amount, such as a mass (e.g., dry mass) or a concentration (e.g., pmol/g). In some embodiments, a first amount and/or a second amount is a mass (e.g., dry mass). In some embodiments, a first amount and/or a second amount is a concentration (e.g., pmol/g). In some embodiments, a first amount and/or a second amount is a relative amount.

In some embodiments, environmental stress comprises, involves, or is related to heat stress, thermal stress, temperature sensitivity-inducing stress, stress as a function of temperature, and the like. In some embodiments, the environmental stress comprises thermal stress.

In some embodiments, the effect of environmental stress is at least one effect selected from the group comprising: reduced health, increased stress, reduced viability, reduced variation, reduced abundance, reduced proliferation, and reduced fecundity.

In some embodiments, the marine invertebrate comprises a holobiont. In some embodiments, the marine invertebrate comprises coral or coral nubbins.

In some embodiments, the biomarker is detected at a level at least 2-fold higher or lower in the sample compared to a control.

In some embodiments, the biomarker is selected from the group comprising: phenylalanine-4-hydroxylase, glycine N-methyltransferase, glyoxylate/hydroxypyruvate reductase, 2′-5′-oligoadenylate synthetase, 4-hydroxyphenylpyruvate dioxygenase, and homogentisate 1,2-dioxygenase.

### Preface to Examples

The devastating loss of coral reefs due to climate change has spurred ‘omics’ research to aid conservation of these valuable, biodiverse ecosystems (Cheung et al., 2021 (the contents of which are herein incorporated by reference in their entirety)). Multi-omics relies on high-throughput approaches such as genomics, transcriptomics, proteomics, and metabolomics to interrogate organismal biology. These methods were developed using data from traditional model organisms, including Arabidopsis, yeast, and Escherichia coli, which often have chromosomal-level genome assemblies and significant knowledge about gene and non-coding region functions, protein-protein interactions (PPI), and complete biochemical pathways based on genetic, bioinformatic, and biochemical data (Jiang et al., 2021 (the contents of which are herein incorporated by reference in their entirety)). Studies of these organisms are also well supported by -omics databases and analysis tools, such as MetaboAnalyst (Xia et al., 2009 (the contents of which are herein incorporated by reference in their entirety)), STRING (Szklarczyk et al., 2019 (the contents of which are herein incorporated by reference in their entirety)), and KEGG (Kanchisa & Goto, 2000 (the contents of which are herein incorporated by reference in their entirety)). These resources allow-omics data relationships to be meaningfully interpreted. Whether multi-omics can be effectively applied to non-model organisms, such as the coral holobiont (whereby individual polyps comprise a cnidarian animal host, a diverse population of large-genome algal symbionts (Symbiodiniaceae), other eukaryotes such as fungi, protists, prokaryotes, and viruses), remains to be determined.

Whereas the coral host of the holobiont inhabits a simple two-tissue body plan (epidermis and gastrodermis, connected by an acellular mesoglea), reefs exist in complex, species-rich, and dynamic marine environments. A useful approach to understand biotic interactions within the coral holobiont is through metabolomics, which is rapidly developing in the coral field. However, the ratio of known to unknown metabolites in corals is still very low when compared to traditional model organisms (Markley et al., 2017 (the contents of which are herein incorporated by reference in their entirety)). The same holds for genomic, transcriptomic, and proteomic data, with many of the genes and proteins identified in corals and their symbionts having unknown functions (Cleves et al., 2020b (the contents of which are herein incorporated by reference in their entirety)), making the interpretation of these-omics data highly challenging.

In recent years, the sea anemone Exaiptasia pallida (also known as Aiptasia) has become a tractable model system for studying holobiont symbioses and stress responses (Baumgarten et al., 2015; Costa et al., 2021; Radecker et al., 2018; Rothig et al., 2016 (the contents of which are herein incorporated by reference in their entirety)). Aiptasia is globally distributed, harbors endosymbiotic Symbiodiniaceae, can be maintained indefinitely in the symbiotic or aposymbiotic (Symbiodiniaceae-free) state, and has a sequenced genome (Baumgarten et al., 2015 (the contents of which are herein incorporated by reference in their entirety)). Because Aiptasia can be propagated sexually and asexually in laboratory tanks, large clonal populations are available for use in high-replicate time-course experiments, and genetic studies (Cleves et al., 2020a (the contents of which are herein incorporated by reference in their entirety)). These characteristics would potentially ameliorate many of the current obstacles in coral-omics data analysis, such as the functional characterization of ‘dark’ (i.e., of unknown function) genes and metabolites, developing metabolic maps specific to cnidarians, and elucidating PPIs. Yet, regardless of the potential of Aiptasia as a Cnidarian model system, this species currently does not have the same resources, background information, or data analysis tools available for multi-omics data analysis and integration as do traditional model organisms. Furthermore, insights from Aiptasia biology cannot always be applied to corals due to the absence of biomineralization in the former, the relatively shorter lifespan (coral colonies can persist for hundreds of years (Kaplan, 2009 (the contents of which are herein incorporated by reference in their entirety))), and a smaller genome size (Baumgarten et al., 2015 (the contents of which are herein incorporated by reference in their entirety)). Thus, understanding the capacity and limitations of -omics techniques applied to the coral holobiont, as well as the cases in which Aiptasia may or may not serve to improve-omics data interpretation, will aid the progress and utility of coral multi-omics research.

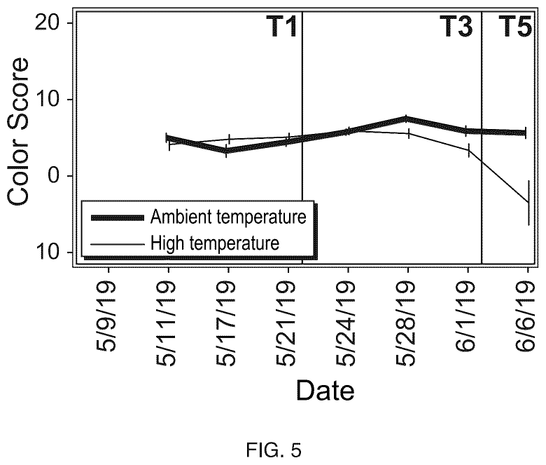

Disclosed herein, novel proteomic and prokaryote microbiome 16S-rRNA amplicon data were analyzed, along with existing transcriptomic and metabolomic data from the stress-resistant Hawaiian coral Montipora capitata (Williams et al., 2021a (the contents of which are herein incorporated by reference in their entirety)). Whereas 16S-rRNA community profiling (e.g., in contrast to prokaryotic metagenomic data) is limited in terms of the questions it can address about changes in the functional ecology of a community, the information gained by this type of analysis provides a useful tool that, in combination with other-omics data, can be used to generate hypotheses for follow-up studies. Profiling of the 16S-rRNA community (presently considered here to be an -omics approach) is also widely used to study the bacterial component of the coral holobiont, therefore understanding how these data can be effectively integrated with other approaches is of high interest. Given these existing data, the present disclosure ascertains how well different layers of multi-omics data can be integrated in M. capitata samples derived from a single experiment (see also Williams et al. 2023). Using the available genome assembly for this coral species as a foundation (Shumaker et al., 2019 (the contents of which are herein incorporated by reference in their entirety)), multiple animal genotypes were subjected to a 5-week thermal stress regime. Control and treatment samples were collected at three time points, which coincide with initial thermal stress, the onset of bleaching, and four days after initial bleaching (FIG. 5) (Williams et al., 2021a (the contents of which are herein incorporated by reference in their entirety)).

The present disclosure finds that transcriptomic and proteomic data broadly capture the thermal stress response of corals, albeit the specific genes identified are often not shared across datasets. The disclosure also finds that whereas the overall magnitude of expression of these datasets are positively correlated, there is significant discordance, which is lessened during stress, in the extent of differential change when comparing control and treatment conditions. In contrast, the metabolite and microbiome data show patterns that likely reflect the complex nature of the holobiont, with these data impacted by homeostatic processes and by fine-scale interactions between the holobiont and its proximate environment. These results provide insights into the different behaviors of multi-omics data and their interpretations when studying complex ecological systems such as corals.

Methods Associated with Examples

### Experimental Design

### Overview of Experimental Design

The methods for M. capitata colony collection, cultivation, and the design of the heat stress experiment are described in Williams et al., 2021a (the contents of which are herein incorporated by reference in their entirety). Briefly, four colonies (genotypes) of M. capitata (designated genotypes MC-206, MC-248, MC-289, and MC-291) were collected from Kãne'ohe Bay, HI, (under Special Administrative Permit (SAP) 2019-60), and fragmented into 30 pieces before being fixed to labeled plugs using hot-glue. The 30 nubbins from each genotype were randomly distributed across tanks that were supplied with a steady flow of water directly from the bay. The temperature of the tanks was controlled by heaters and lights were used to simulate a 12-hour light/12-hour dark cycle. The nubbins were left to acclimate at ambient temperature (˜27° C.) for 5 days before the high-temperature treatment tanks were increased by ˜0.4° C. every 2 days for a total of 9 days, until they were between 30.5-31.0° C. The treatment (hereinafter, high temperature) tanks were held at ˜30.5° C. and the control (hereinafter, ambient temperature) tanks at ˜ 27.5° C. until the end of the experiment, which lasted an additional 16 days. The temperature of 30.5° C. was chosen for thermal stress because it is the expected range for natural warming events in Káne'ohe Bay (Jokiel & Coles, 1990 (the contents of which are herein incorporated by reference in their entirety)). Three nubbins per genotype (n=3 replicates) were collected at five time points (T1-T5) during the experiment, however, only samples from T1 (after temperature ramp-up was complete), T3 (at the onset of bleaching; 13 days after T1), and T5 (on the last day of the experimental period; 17 days after T1) were processed for multi-omics analysis. Bleaching progression was monitored using color scores (Siebeck et al., 2006 (the contents of which are herein incorporated by reference in their entirety)) generated for the ambient and stress treated nubbins at each of the five time points (FIG. 5) (Williams et al., 2021a (the contents of which are herein incorporated by reference in their entirety)). The samples collected at each time point were flash-frozen in liquid N2 and stored at −80° C.; each frozen sample was divided into subsamples which were processed for a different multi-omics method. Three nubbins were also collected from the same coral colonies in Kãne'ohe Bay (hereinafter, field samples), flash frozen in liquid N2, and stored at −80° C. These frozen field samples were processed for multi-omics using the same protocols applied to the time-point samples. Samples from all four colonies/genotypes were used for metabolomics (published in Williams et al., 2021a (the contents of which are herein incorporated by reference in their entirety)) and 16S-rRNA microbiome analysis (disclosed herein); samples from colony MC-289 were used for RNA sequencing (published in Williams et al., 2021b (the contents of which are herein incorporated by reference in their entirety)) and proteomic analysis (disclosed herein).

Consideration with Respect to Experimental Design

Experimental design is integral to the successful utilization of multi-omics data; whereas large samples size is generally seen as a requirement, smaller sample sizes should not be viewed as a weakness in all cases. Although the number of samples included in the analysis of the present disclosure was small, nubbins from a controlled set of coral colonies were prioritized (i.e., a limited set of coral genotypes) to mute the impact of genotype on multi-omics data (Grottoli et al., 2021 (the contents of which are herein incorporated by reference in their entirety)). Furthermore, the unintentional sequencing of two samples with different genotypes demonstrates the effect of genotype on-omics data, particularly proteomic data. Samples from the same genotype show limited variation in the proteome data when compared to the single sample from a different genotype (also observed by Mayfield et al., 2021 (the contents of which are herein incorporated by reference in their entirety)). In contrast, samples from different genotypes often (but not always) have higher than expected variation in the transcriptomic data, but this is masked by the greater overall change in these datasets. These results are consistent with the idea that different-omics datasets have very different dynamics, specifically, proteomic data are under homeostatic constraints and change very little, whereas transcriptomic data are far more impacted by local environmental shifts. Additionally, given that there are practical and regulatory restrictions on the size of samples that can be collected from coral colonies, there was a limit on the number of nubbins which could be generated from each colony, and therefore how many samples from which data could be generated. This is particularly true for multi-omics studies, which require all-omics data to be derived from the same samples (Tarazona et al., 2018 (the contents of which are herein incorporated by reference in their entirety)), therefore nubbins must be large enough to allow for extraction of DNA, RNA, metabolites, and/or proteins. Small, highly controlled experiments, such as presented here with MC-289, allow for the same genotypes to be tracked across the treatments and time points, providing a useful platform to assess the different data types (i.e., free from any genotypic affects).

### Selected Data Analysis Methods

### PCA, PCOA, and PERMANOVA

Principal component analysis (PCA), principal coordinate analysis (PCoA), and permutational multivariate analysis of variance (PERMANOVA) were performed on the normalized metabolomic, transcriptomic (cumulative TPM>100), proteomic, and 16S (prokaryotic ribosomal RNA) microbiome datasets. PCA was done using the ‘prcomp’ function (center=FALSE, scale=FALSE) from the R statistical functions package stats v4.1.2. PERMANOVA tests were conducted on Bray-Curtis dissimilarity matrices using the ‘adonis2’ (permutations =999, method=‘bray’; using replicate tank as the strata) and ‘vegdist’ (method=‘bray’) functions in the vegan v2.6-2 ecology R package (Oksanen et al., 2020 (the contents of which are herein incorporated by reference in their entirety)). Only 190 permutations were used for the metabolomics PERMANOVA as this was the maximum value possible given the smaller number of samples in the dataset. When analyzing just the M. capitata genotype MC-289 samples, the PERMANOVA formula “TimePoint*Treatment” was used; when analyzing all genotypes, the formula “TimePoint*Treatment*Genotype” was used. It should be noted that the most significant p-value produced by this analysis is p=0.001. PCoA was performed on the Bray-Curtis dissimilarity matrix using ‘wcmdscale’ function (k=2, eig=TRUE, add=“cailliez”) from vegan v2.6-2 (Oksanen et al., 2020 (the contents of which are herein incorporated by reference in their entirety)).

### Sparse PLS-DA

The machine-learning method, partial least squares-discriminant analysis (PLS-DA), was performed on the normalized metabolomic, transcriptomic (cumulative TPM>100), and proteomic datasets using the ‘splsda’ function (ncomp=6, scale=FALSE, near.zero. var=TRUE) from the multivariate methods R package mixOmics v6.18.1 (Rohart et al., 2017). For the 16S microbiome dataset, the ‘perf’ function was used to evaluate the performance of PLS-DA using repeated k-fold cross-validation (validation=“Mfold”, nrepeat=50). The ‘tune’ function was then applied to determine the number of variables (1-1000) to select on each component for sparse PLS-DA (dist=‘max.dist’, measure=“BER”, nrepeat=50), before the ‘splsda’ function was run with the tuned number of components and variables per component.

### Proteome and Transcriptome Methods

### Proteomic Data

Proteomic data were generated for M. capitata genotype MC-289 from two out of the three replicate nubbins per time point (T1, T3, and T5) per condition (including field samples). The proteins were extracted using a protocol adapted from Stuhr et al., 2018 (the contents of which are herein incorporated by reference in their entirety). The lysis buffer comprised 50 mM Tris-HCl (pH 7.8), 150 mM NaCl, 1% sodium dodecyl sulfate (SDS), and complete, Mini EDTA-free Tablets (protease inhibitor). One gram of each sample was ground in a mortar on ice with 100 μl of lysis buffer. The sample was then transferred to a 2 mL Eppendorf tube, with an additional 50 μl of lysis buffer used to wash the mortar, for a total lysate volume of 150 μl. Each sample was vortexed for 1 min, stored on ice for 30 min, and clarified by centrifugation at 10,000 rcf for 10 minutes (4° C.). Protein concentrations were measured using the Pierce 660-nm Protein Assay. Thereafter, 40 μg of each sample was run on an SDS-polyacrylamide gel electrophoresis (PAGE) gel, with slices collected and incubated at 60° C. for 30 minutes in 10 mM Dithiothreitol (DTT). After cooling to room temperature, 20 mM iodoacetamide was added to the gel slices before they were kept in the dark for 1 hour to block free cysteine. The samples were digested using trypsin at a concentration of 1:50 (weight: weight, trypsin: sample) before being incubated at 37° C. overnight. The digested peptides were dried under vacuum and washed with 50% acetonitrile to PH neutral. The digested peptides were labeled with Tandem Mass Tag 6-plex (TMT6plex) (Thermo Fisher Scientific, Waltham, MA, USA; Lot #: UF288619) following the manufacturer's protocol, before being pooled together at a 1:1 ratio. The pooled samples were dried and desalted with solid phase extraction cartridge (SPEC) Pt C18 (Agilent Technologies, Santa Clara, CA, USA, Catalog #: A57203) before fractionation using an Agilent 1100 series machine. The samples were solubilized in 250 μl of 20 mM ammonium (pH 10) and injected onto an XBRIDGE® column (Waters Corporation, Milford, MA, USA; C18 3.5 μm 2.1 ×150 mm) using a linear gradient of 1% buffer B/min from 2-45% of buffer B (buffer B: 20 mM ammonium in 90% acetonitrile, pH 10). Ultraviolet (UV) absorbance at 214 nm was monitored while fractions were collected. Each fraction was desalted (Rappsilber et al., 2007 (the contents of which are herein incorporated by reference in their entirety)) and analyzed by liquid chromatography-tandem mass spectrometry (LC-MS/MS).

Nano-LC-MS/MS was performed using a DIONEX® rapid-separation liquid chromatography system interfaced with an ORBITRAP® Eclipse mass spectrometer (Thermo Fisher Scientific). Selected desalted fractions 28-45 were loaded onto an Acclaim PepMap 100 trap column (75 μm×2 cm, Thermo Fisher Scientific) and washed with 0.1% trifluoroacetic acid for 5 minutes with a flow rate of 5 μl/min. The trap was brought in-line with the nano analytical column (nanoEase M/Z peptide BEH C18, 130 Å, 1.7 μm, 75 μm×20 cm, Waters Corporation) with a flow rate of 300 nL/min using a multistep gradient: 4% to 15% of 0.16% formic acid and 80% acetonitrile in 20 minutes, then 15%-25% of the same buffer in 40 minutes, followed by 25%-50% of the buffer in 30 minutes. The scan sequence began with a first stage (MS¹) spectrum (ORBITRAP® analysis, resolution 120,000, scan range from 350-1600 Th, automatic gain control (AGC) target 1E6, maximum injection time 100 ms). For synchronous precursor selection for three-stage mass spectrometry (SPS3), MS/MS analysis consisted of collision-induced dissociation (CID), quadrupole ion trap analysis, automatic gain control (AGC) 2E4, normalized collision energy (NCE) 35, maximum injection time 55 ms, and isolation window at 0.7 atomic mass units (amu). Following acquisition of each second-stage (MS²) spectrum, a third-stage (MS³) spectrum was collected in which 10 MS² fragment ions were captured in the MS³ precursor population using isolation waveforms with multiple frequency notches. MS³ precursors were fragmented by higher-energy collisional dissociation (HCD) and analyzed using the ORBITRAP® (NCE 55, AGC 1.5E5, maximum injection time 150 ms, resolution was 50,000 at 400 Th scan range 100-500). The whole cycle was repeated for 3 seconds before repeating from an MS¹ spectrum. Dynamic exclusion of 1 repeat and duration of 60 sec was used to reduce the repeat sampling of peptides. LC-MS/MS data were analyzed with Proteome Discoverer 2.4 (Thermo Fisher Scientific) with the sequence search engine run against the protein sequences of the genes predicted in the published M. capitata genome (Shumaker et al., 2019 (the contents of which are herein incorporated by reference in their entirety)) and a database that consisted of common lab contaminants. The MS mass tolerance was set at ±10 ppm and MS/MS mass tolerance was set at ±0.4 Da for the proteome. Protein tandem mass tag (TMTpro) on C- and N-terminus of peptides and carbamidomethyl on cysteine (CAM) were set as static modifications. Methionine oxidation, protein N-terminal acetylation, protein N-terminal methionine loss, or protein N-terminal methionine loss plus acetylation were set as dynamic modifications for proteome data. Peptide-identification algorithm Percolator (Käll et al., 2007 and The et al., 2016 (the contents of which are herein incorporated by reference in their entirety)) was used for results validation. A concatenated reverse database was used for the target-decoy strategy.

For reporter ion quantification, the reporter abundance was set to use the signal/noise ratio (S/N) only if all spectrum files had S/N values, otherwise, intensities were used instead of S/N values. The ‘quant’ value was corrected for isotopic impurity of reporter ions. The co-isolation threshold was set at 50%. The average reporter S/N threshold was set to 10 and the SPS mass matches percent threshold was set to 65%. The protein abundance of each channel was calculated using summed S/N of all unique and razor peptides. Finally, the abundance was further normalized to a summed abundance value for each channel over all peptides identified within a file. Only peptide sequences from genes predicted in the M. capitata genome were used in the present disclosure. Proteins with a false discovery rate (FDR)<0.01 were considered “high” confidence, and proteins with a FDR ≥0.01 but <0.05 were considered “medium” confidence. Differentially expressed proteins (DEPs; log₂FC >1, p-value <0.05) were identified between the ambient and high temperature treatments for each time point using the stats v4.1.2 and mixOmics v6.18.1 R packages (Rohart et al., 2017 (the contents of which are herein incorporated by reference in their entirety)). Adjusted p-values were not used for this analysis due to the low number of replicates per condition.

### Transcriptomic Data

RNA-seq data was generated for M. capitata genotype MC-289 across the three analyzed time points, two treatment conditions, and field colony (n=3 replicates each). The methods for cDNA library preparation, sequencing, and data analysis are detailed in Williams et al., 2021b (the contents of which are herein incorporated by reference in their entirety). Briefly, a Qiagen ALLPREP® DNA/RNA/miRNA Universal Kit (Qiagen, Germantown, MD, USA) was used to extract RNA from the (crushed) frozen samples; a TRUSEQ® RNA Sample Preparation Kit v2 (Illumina, San Diego, CA, USA) was used to generate strand specific cDNA libraries that were sequenced on a NOVASEQ® machine (Illumina) (2×150 bp flow cell). This protocol included a poly-A selection step, which enriched for transcripts from eukaryotic cells and depleted those from the prokaryotic microbiome. RNA-seq reads were trimmed for low quality bases and adapters using Trimmomatic v0.38 (Bolger et al., 2014 (the contents of which are herein incorporated by reference in their entirety)); read pairs where both mates survived trimming were used to quantify the expression levels of the genes predicted in the M. capitata genome (Shumaker et al., 2019 (the contents of which are herein incorporated by reference in their entirety)) using Salmon v1.10 (Patro et al., 2017 (the contents of which are herein incorporated by reference in their entirety)). Differentially expressed genes (DEGs; log₂FC >1, adjusted p-value <0.05) were identified between the ambient and high temperature treatments at each time point by the DESeq2 v1.34.0 R package (Love et al., 2014 (the contents of which are herein incorporated by reference in their entirety)) using the aligned read counts produced by Salmon. The Transcripts Per Million (TPM)-normalized expression values produced by Salmon were used for all downstream visualization and ordination analyses. Transcripts with a cumulative TPM>100 (i.e., >100 TPM summed across all samples) were used for the ordination and statistical analysis.

### Gene Functional Annotation

Functional assignment of the M. capitata proteins was done using functional annotation tool eggNOG-mapper (v2.1.6;--pfam_realign denovo; database release 2021 Dec. 9) (Cantalapiedra et al., 2021; Huerta-Cepas et al., 2019 (the contents of which are herein incorporated by reference in their entirety)) and a DIAMOND protein aligner search (v2.0.15; blastp--ultra-sensitive--max-target-seqs 1000) (Buchfink et al., 2021 (the contents of which are herein incorporated by reference in their entirety)) against the National Center for Biotechnology Information (NCBI) non-redundant (nr) database (release 2022_07). eggNOG-mapper was also used to assign KEGG orthologous numbers (KO numbers).

### Proportion of Shared SNPs Between Transcriptome Samples

The proportion of single nucleotide polymorphisms (SNPs) shared between each pairwise combination of transcriptome samples was used to confirm that they were all derived from the same colony (genotype). Each sample was aligned against the M. capitata reference genome (Shumaker et al., 2019 (the contents of which are herein incorporated by reference in their entirety)) using transcript alignment tool STAR (v2.7.8a;--sjdbOverhang 149--twopassMode Basic) (Dobin et al., 2013 (the contents of which are herein incorporated by reference in their entirety)). Read-group information was extracted from the read names using read-group inference tool rgsam (v0.1; github.com/djhshih/rgsam;--qnformat illumina-1.8) and added to the aligned reads using genome analysis toolkit gatk ‘FastqToSam’ and gatk ‘MergeBamAlignment’ (--INCLUDE_SECONDARY_ALIGNMENTS false--VALIDATION_STRINGENCY SILENT). Duplicate reads were removed using gatk ‘MarkDuplicates’ (--VALIDATION_STRINGENCY SILENT) before reads that spanned intron-exon boundaries were split using gatk ‘SplitNCigarReads’ (default). Haplotypes were called using gatk ‘HaplotypeCaller’ (--dont-use-soft-clipped-bases-ERC GVCF), with the resulting genomic variant call format (GVCF) files (one per sample) combined using gatk ‘CombineGVCFs’ before being jointly genotyped using gatk ‘GenotypeGVCFs’ (-stand-call-conf 30) (Poplin et al., 2018 (the contents of which are herein incorporated by reference in their entirety)). The resulting variant were filtered for indels, sites with low average reads coverage across all samples, and sites without called genotypes across all samples using variant call format (VCF) package VCFtools (v0.1.17;--remove-indels--min-meanDP 10--max-missing 1.0) (Danecek et al., 2011 (the contents of which are herein incorporated by reference in their entirety)). The “vcf_clone_detect.py” script (from github.com/pimbongaerts/radseq; retrieved Jun. 12, 2021) was used with the filtered variants to compute the number of SNPs shared between each pair of samples.

### Protein Abundance-Transcript Expression Level Correlation Methods

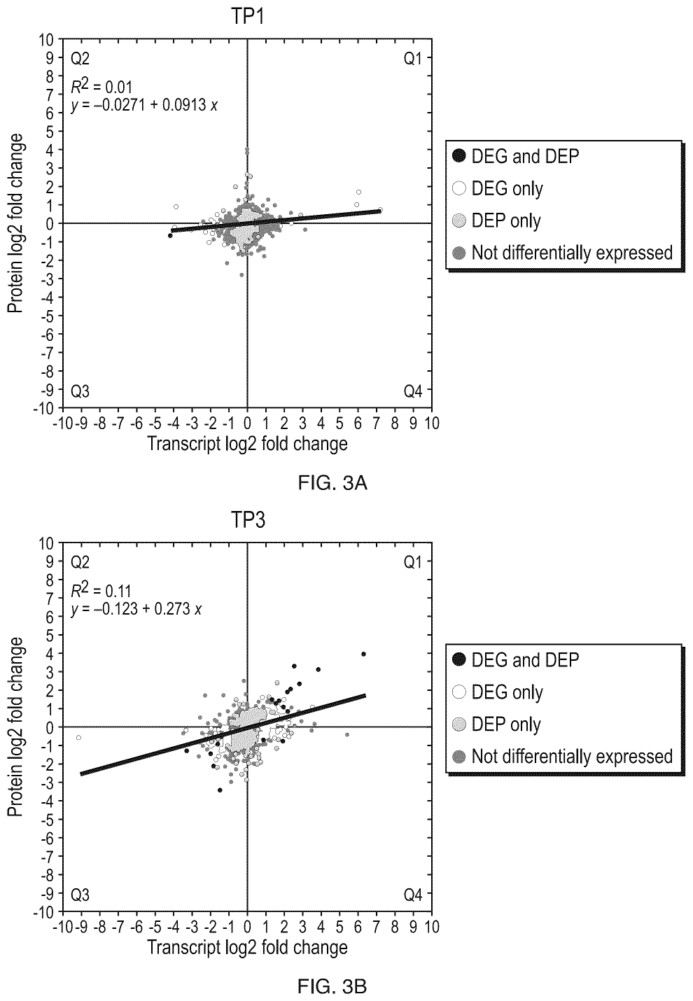

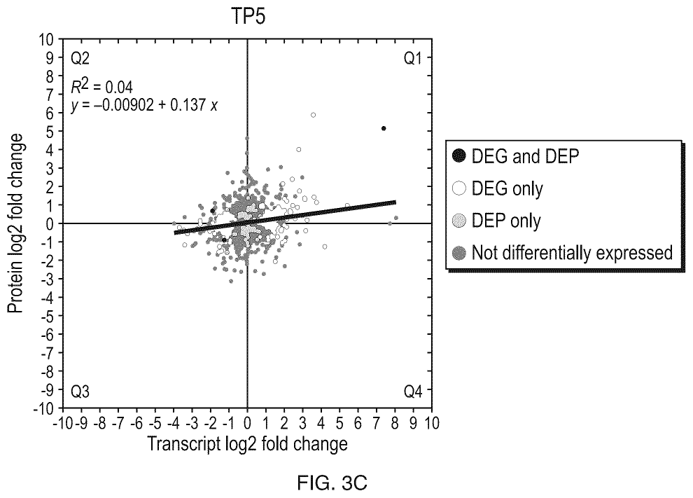

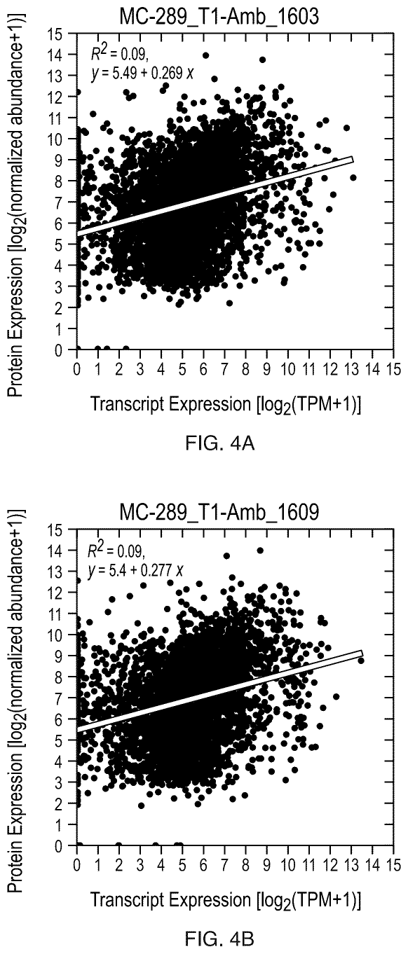

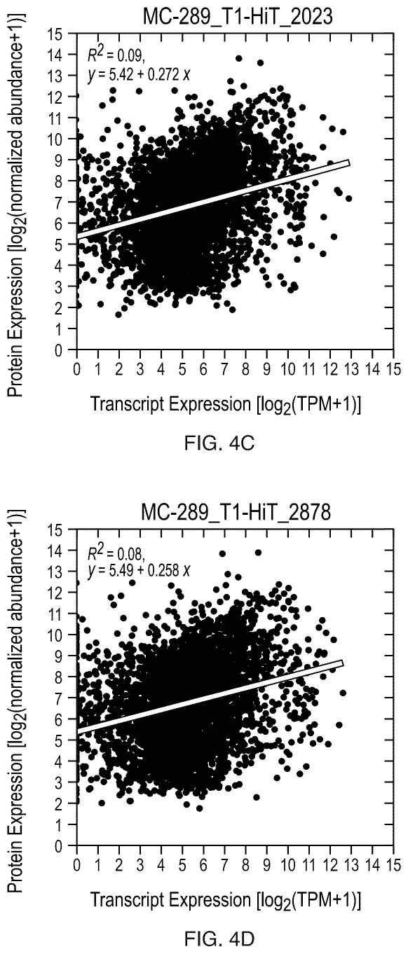

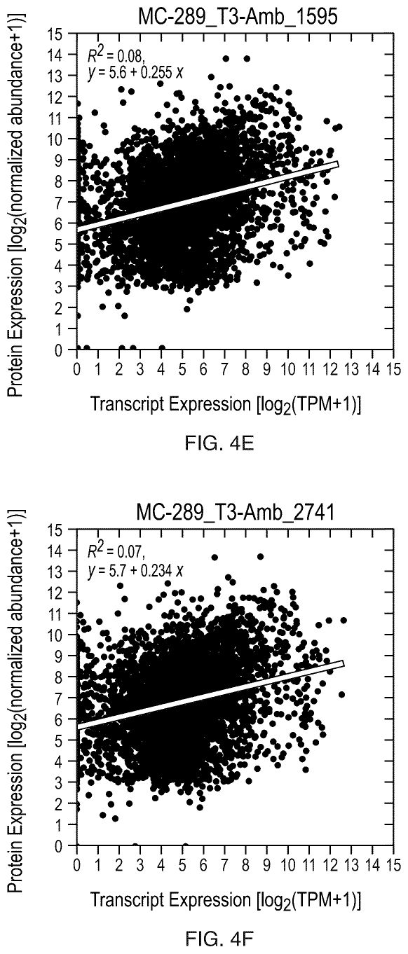

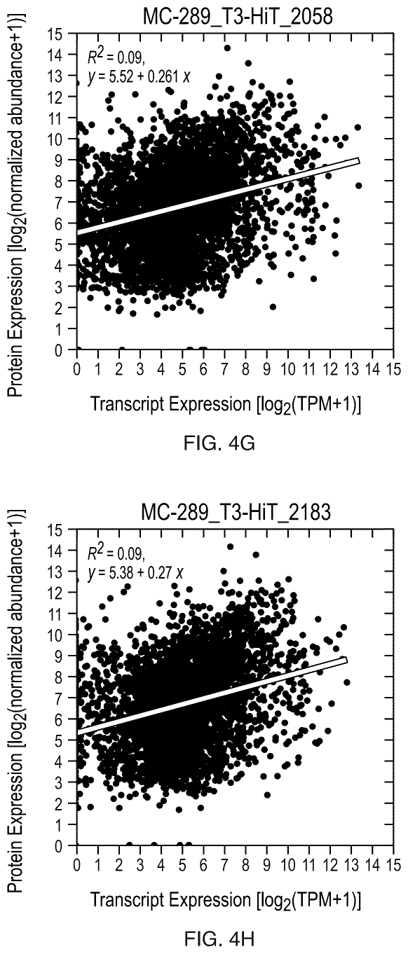

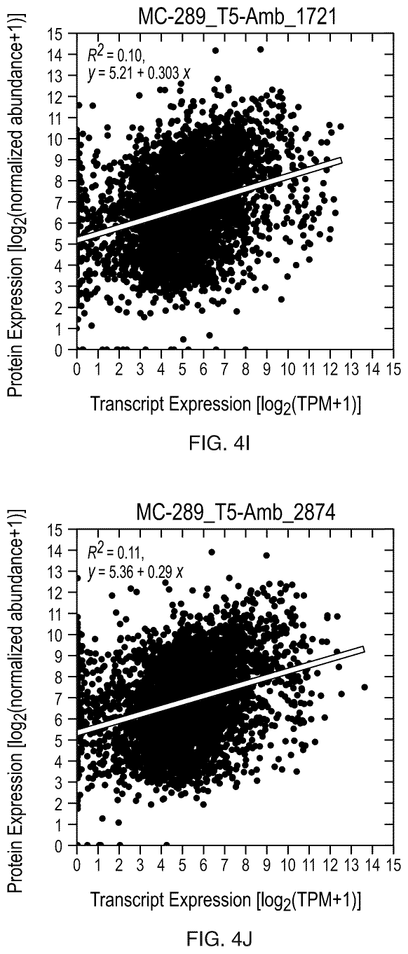

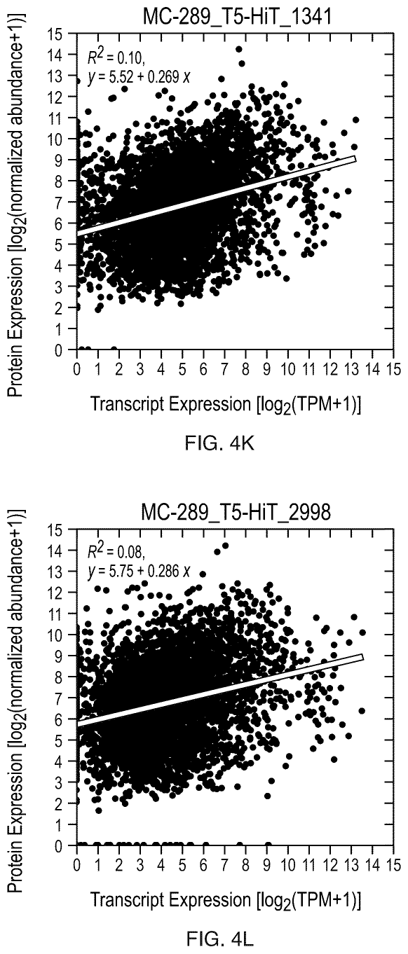

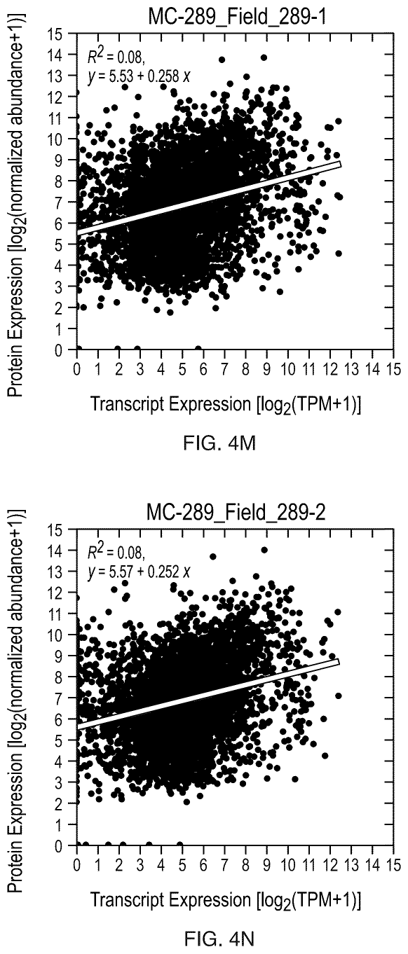

The correlation between gene expression and protein abundance was assessed using the samples from M. capitata genotype MC-289. Only genes (n=4036) which were detected in the proteome data of at least one sample were used in this analysis. The transcripts per kilobase million (TPM)-normalized gene expression and abundance-normalized protein abundance values were scaled using a log₂ transformation (with an offset of 1 to prevent infinite log values) before being plotted (FIGS. 4A-4N). The magnitude and directionality of the stress response of the genes detected in the proteome data was assessed using the log₂ (fold-change) (log₂FC) values computed during differential transcriptome and proteome expression analysis (FIGS. 3A-3C).

### Polar Metabolomic Data Methods

### Polar Metabolomic Data

Polar metabolite data were generated for each of the four genotypes (MC-206, MC-248, MC-289, MC-291) across the three analyzed time points (T1, T3, T5), two treatment conditions (ambient (Amb) or high (HiT) temperature), and field colonies (n=3 replicates each). The methods used for polar metabolite extraction and analysis are described in Williams et al., 2021a (the contents of which are herein incorporated by reference in their entirety). Briefly, metabolites were extracted from each sample with a 40:40:20 (v/v/v) extraction buffer (MeOH: acetonitrile:H₂O+Formic Acid) and followed a protocol optimized for the extraction of water-soluble polar metabolites; the resulting metabolite extracts were separated into phases using hydrophilic interaction liquid chromatography (performed on a Vanquish Horizon ultra-high performance liquid chromatography (UHPLC) system, Thermo Scientific, Waltham, MA, USA). A Thermo Fisher Scientific Q EXACTIVER Plus mass spectrometer was used for the full-scan MS analysis and to generate the MS² spectra. The resulting metabolite data was analyzed using LS-MS data-processing tool ElMaven (Agrawal et al., 2019 (the contents of which are herein incorporated by reference in their entirety)). Peaks that had ion counts above 50,000 (before normalization) were retained. The metabolite profiles for all samples were aligned using Ordered Bijective Interpolated Warping (OBI-Warp) (Prince & Marcotte 2006 (the contents of which are herein incorporated by reference in their entirety)). Metabolites in the resulting list were filtered, retaining only those with 48 “good peaks” (as defined/called by OBI-Warp) and a ‘max Quality’ score of 0.8. The metabolite intensities were normalized using the frozen weights of each sample. These filtered and normalized peaks were used for downstream analysis and to generate total metabolite counts.

### Dipeptide Quantification

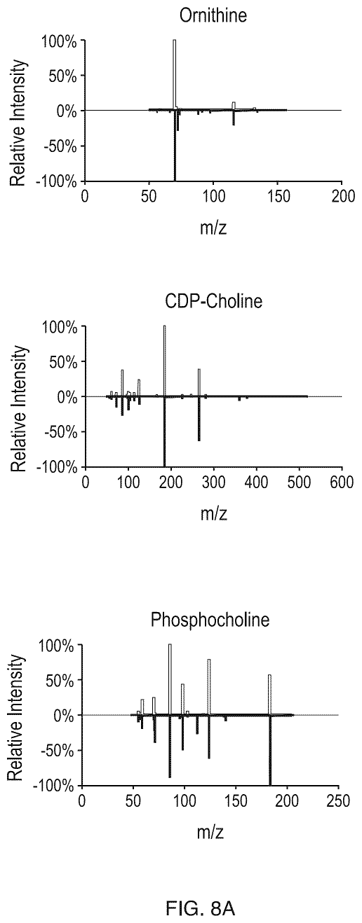

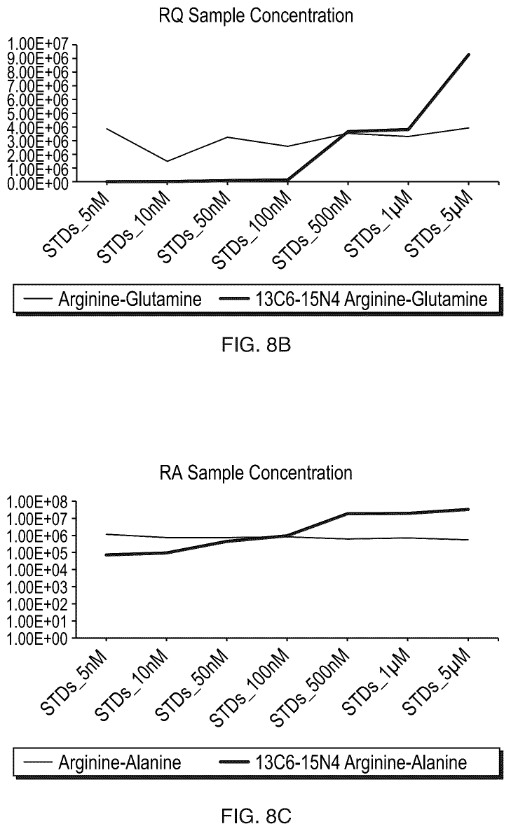

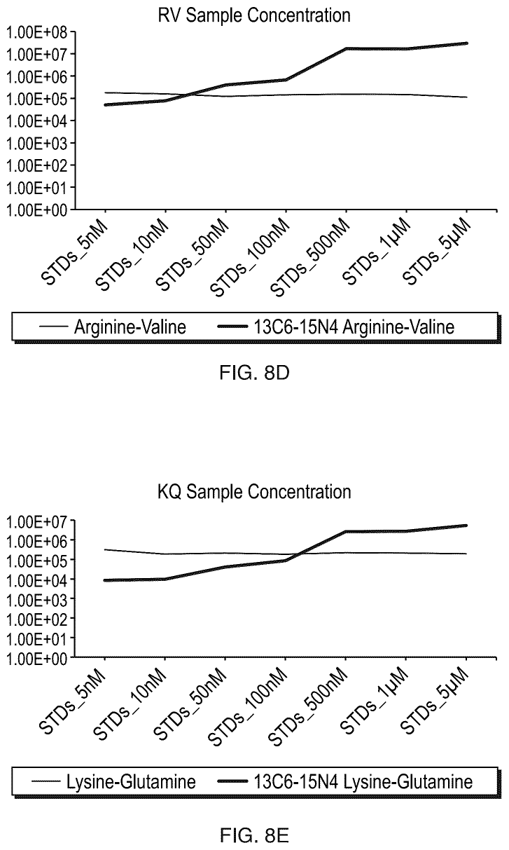

Dipeptide metabolite calls made previously using MS¹ full scans from coral samples were verified using parallel reaction monitoring (PRM) second stage mass spectrum (MS²) spectral patterns from the same samples. These spectra were then compared to the high quality MS² spectra derived from pure chemical standards of the analogous metabolites. The spectral fragments from the samples were then assessed for their correlation to the signal of the parental masses. Fragments with low correlation were removed. The trimmed spectral patterns were normalized to the highest signal intensity and subsequently compared to the spectral patterns of the chemical standards, also normalized, for qualitative relatedness (FIG. 8A). A master mix of the stable isotope labeled dipeptide internal standards (IS) were made at uniform concentrations. The IS were then spiked into the pooled sample so that the concentration in the sample would be as follows: 5 nM, 10 nM, 50 nM, 100 nM, 500 nM, 1 μM, and 5 μM. These samples were run under positive polarity and the signal of the endogenous dipeptide level was compared to that of the labeled IS. The point of intersection of the traces identifies the concentration of the endogenous dipeptide level in the samples (FIGS. 8B-8E).

Using the result of the initial concentration curve of the labeled samples, a master mix of the stable isotope labeled dipeptide internal standards (IS) were remade at a more physiologically relevant concentration (arginine-glutamine (RQ), 500 nM; arginine-alanine (RA) and lysine-glutamine (KQ), 100 nM; arginine-valine (RV), 10 nM). A master mix of all the standards at a 10× higher concentration than desired in the samples was prepared and then diluted 1:10 in the sample vial. Quantification experiments included an ambient temperature control at the same time point (8 weeks) and corresponding samples of the Pocillopora acuta (P. acuta) species.

Microbiome 16S rRNA Methods

Microbiome V3-V4 hypervariable region 16S-ribosomal RNA (rRNA) sequencing data were generated for each of the four genotypes (MC-206, MC-248, MC-289, MC-291) across the three analyzed time points (T1, T3, T5), two treatment conditions (ambient (Amb) or high (HiT) temperature), and for the field colonies (n=3 replicates each). The cells in each sample were lysed using liquid nitrogen and mechanical grinding. Total DNA was isolated using a Qiagen ALLPREP® DNA/RNA/miRNA Universal Kit, following the manufacturer's instructions. The 16S-rRNA amplicon sequencing libraries were prepared as per Illumina's instructions (Illumina, 2013), using primers designed for the V3 and V4 hypervariable region, the NEXTERA® XT library preparation kit (Illumina), and dual indexes (i7 and i5). The libraries were pooled together and a 20% PhiX control library spike-in was added. Quality control was performed using a QUBIT® fluorometer (Invitrogen, Waltham, MA, USA) and an Agilent BIOANALYZER®, with the target library length being ˜600 bp. Libraries were sequenced by Genewiz (South Plainfield, NJ, USA) on an Illumina MiSeq machine (2×300 bp flow cell). Raw reads were trimmed for quality and removal of primer sequence using Cutadapt (Martin, 2011). Quality trimming and filtering, denoising, merging and chimera removal, and amplicon sequence variant [ASV] feature table construction were carried out using the QIIME 2 2021.4 plug-in (Bolyen et al., 2019 (the contents of which are herein incorporated by reference in their entirety)) for sample inference tool DADA2 (Callahan et al., 2016 (the contents of which are herein incorporated by reference in their entirety)). Taxonomic assignment was carried out with QIIME2 against the SILVA 16S-rRNA database (release 138) (Quast et al., 2013 (the contents of which are herein incorporated by reference in their entirety)). The initial ASV feature table derived from the trimmed reads was filtered, removing ASV that were: (1) too short (<390 bases); (2) did not have unambiguous taxonomic assignments to at least the phylum-level; (3) had taxonomic assignments of “Archaea”, “Chloroplast” or “Mitochondria”; and (4) had a frequency of <20 reads across all 83 samples. The <20 reads cutoff was chosen based on similar filtering approaches deployed in other studies (Doering et al., 2021 (the contents of which are herein incorporated by reference in their entirety)). These studies, which often consisted of <30 samples, used cutoffs of <10 reads; therefore, to account for the larger number of samples in the analysis, a cutoff of 20 reads was chosen. Per-sample ASV counts were rarefied prior to α- and β-diversity analysis. Shapiro-Wilk tests of Shannon and Simpson α-diversity metrics show that the data are non-normal (p-value <0.01; Table 5). Analyses of α- and β-diversity were carried out in R using high-throughput phylogenetic sequence data analysis package phyloseq (McMurdie & Holmes, 2013 (the contents of which are herein incorporated by reference in their entirety)), statistical functions package stats, and ecology package vegan (Oksanen et al., 2020 (the contents of which are herein incorporated by reference in their entirety)). To visualize β-diversity, samples were rarefied to 39,902 reads per sample (chosen by rarefaction analysis to minimize loss of data) using the ‘rarefy_even_depth’ function in the phyloseq R package (McMurdie & Holmes, 2013 (the contents of which are herein incorporated by reference in their entirety)), after which the Bray-Curtis distances between samples were calculated using the ‘distance’ function.

The psych v2.2.3 R package (Revelle, 2022 (the contents of which are herein incorporated by reference in their entirety)) was used to determine whether significant correlations exist between the 16S-rRNA amplicon and metabolite data. Amplicon count data were agglomerated by taxon at multiple taxonomic ranks—from phylum to genus—for testing using the ‘tax_glom’ function within the phyloseq package. Pairwise correlations using the Spearman method, as well as adjustment of p-values using the Benjamini-Hochberg method, were performed on both raw and normalized (by relative abundance) quantifications using the ‘corr.test’ function. Correlations were retained for further analysis if the associated adjusted p-value was less than 0.05. Furthermore, because Spearman rank correlation analyses are sensitive to low values, only taxa with greater than 200 observations across all samples were considered. Correlations were visualized with correlation plots and histograms, generated using the chart function in the PerformanceAnalytics v2.0.4 R package (Brian G. Peterson et al., 2022 (the contents of which are herein incorporated by reference in their entirety)).

### Conclusion to Findings Presented in Examples

In summary, transcriptomic and proteomic data are weakly positively correlated and provide useful (albeit, often conflicting) insights into coral biology (National Academies of Sciences & Medicine, 2019 (the contents of which are herein incorporated by reference in their entirety)). Metabolomics data, which assesses intermediates and end products of cellular regulatory processes, suffers from limited knowledge about the diversity of cnidarian metabolites and complex turnover processes (i.e., production vs. utilization). This aspect makes the results presented in this disclosure more challenging to interpret and integrate with other-omics data, although stress markers which demonstrate consistent correlation with stress (e.g., proteins and dipeptides) have been identified. The usefulness of the M. capitata coral microbiome amplicon data is less obvious and will require coral specific databases and other types of -omics analysis (Keller-Costa et al., 2021 (the contents of which are herein incorporated by reference in their entirety)) to provide the needed insights.

The present disclosure leads to three major conclusions about coral multi-omics data. Firstly, it is critical to constrain experiments with respect to genotype and treatment conditions to minimize genetic or stochastic variation in-omics data. This applies particularly to the metabolomic and microbiome analyses, because these data show a more complex pattern of variation. Secondly, there is an urgent need for high-quality reference genomes for all members of the holobiont to facilitate analysis of meta-transcriptome and meta-genome data to elucidate biotic interactions. Thirdly, these experiments need to be extended to multiple coral species and may vary in how informative the -omics layers will be about fundamental processes due to differences in the underlying genetic structure, holobiont composition, and local adaptation of lineages.

### EXEMPLIFICATION

### Example 1. Proteome and Transcriptome Data

### Proteomic Data Method

Proteomic data were generated for M. capitata genotype MC-289 from two out of the three replicate nubbins per time point (T1, T3, and T5) per condition (including field samples). The proteins were extracted using a protocol adapted from Stuhr et al., 2018 (the contents of which are herein incorporated by reference in their entirety). The lysis buffer comprised 50 mM Tris-HCl (pH 7.8), 150 mM NaCl, 1% SDS, and complete, Mini EDTA-free Tablets. One gram of each sample was ground in a mortar on ice with 100 μl of lysis buffer. The sample was then transferred to a 2 mL Eppendorf tube, with an additional 50 μl of lysis buffer used to wash the mortar, for a total lysate volume of 150 μl. Each sample was vortexed for 1 min, stored on ice for 30 min, and clarified by centrifugation at 10,000 ref for 10 minutes (4° C.). Protein concentrations were measured using the Pierce 660-nm Protein Assay. Thereafter, 40 μg of each sample was run on an SDS-PAGE gel, with slices collected and incubated at 60° C. for 30 minutes in 10 mM Dithiothreitol (DTT). After cooling to room temperature, 20 mM iodoacetamide was added to the gel slices before they were kept in the dark for 1 hour to block free cysteine. The samples were digested using trypsin at a concentration of 1:50 (weight: weight, trypsin: sample) before being incubated at 37° C. overnight. The digested peptides were dried under vacuum and washed with 50% acetonitrile to PH neutral. The digested peptides were labeled with Tandem Mass Tag 6-plex (TMT6plex) (Thermo Fisher Scientific, Waltham, MA, USA; Lot #: UF288619) following the manufacturer's protocol, before being pooled together at a 1:1 ratio. The pooled samples were dried and desalted with solid phase extraction cartridge (SPEC) Pt C18 (Agilent Technologies, Santa Clara, CA, USA, Catalog #: A57203) before fractionation using an Agilent 1100 series machine. The samples were solubilized in 250 μl of 20 mM ammonium (pH 10) and injected onto an XBRIDGE® column (Waters Corporation, Milford, MA, USA; C18 3.5 μm 2.1×150 mm) using a linear gradient of 1% buffer B/min from 2-45% of buffer B (buffer B: 20 mM ammonium in 90% acetonitrile, pH 10). Ultraviolet (UV) absorbance at 214 nm was monitored while fractions were collected. Each fraction was desalted (Rappsilber et al., 2007 (the contents of which are herein incorporated by reference in their entirety)) and analyzed by liquid chromatography-tandem mass spectrometry (LC-MS/MS).

Nano-LC-MS/MS was performed using a DIONEX® rapid-separation liquid chromatography system interfaced with an ORBITRAP® Eclipse mass spectrometer (Thermo Fisher Scientific). Selected desalted fractions 28-45 were loaded onto an Acclaim PepMap 100 trap column (75 μm×2 cm, Thermo Fisher Scientific) and washed with 0.1% trifluoroacetic acid for 5 minutes with a flow rate of 5 μl/min. The trap was brought in-line with the nano analytical column (nanoEase M/Z peptide BEH C18, 130 Å, 1.7 μm, 75 μm×20 cm, Waters Corporation) with a flow rate of 300 nL/min using a multistep gradient: 4% to 15% of 0.16% formic acid and 80% acetonitrile in 20 minutes, then 15%-25% of the same buffer in 40 minutes, followed by 25%-50% of the buffer in 30 minutes. The scan sequence began with a first stage (MS¹) spectrum (ORBITRAP® analysis, resolution 120,000, scan range from 350-1600 Th, automatic gain control (AGC) target 1E6, maximum injection time 100 ms). For synchronous precursor selection for three-stage mass spectrometry (SPS3), MS/MS analysis consisted of collision-induced dissociation (CID), quadrupole ion trap analysis, automatic gain control (AGC) 2E4, normalized collision energy (NCE) 35, maximum injection time 55 ms, and isolation window at 0.7 atomic mass units (amu). Following acquisition of each second-stage (MS²) spectrum, a third-stage (MS³) spectrum was collected in which 10 MS² fragment ions were captured in the MS³ precursor population using isolation waveforms with multiple frequency notches. MS³ precursors were fragmented by higher-energy collisional dissociation (HCD) and analyzed using the ORBITRAP® (NCE 55, AGC 1.5E5, maximum injection time 150 ms, resolution was 50,000 at 400 Th scan range 100-500). The whole cycle was repeated for 3 seconds before repeating from an MS¹ spectrum. Dynamic exclusion of 1 repeat and duration of 60 sec was used to reduce the repeat sampling of peptides. LC-MS/MS data were analyzed with Proteome Discoverer 2.4 (Thermo Fisher Scientific) with the sequence search engine run against the protein sequences of the genes predicted in the published M. capitata genome (Shumaker et al., 2019 (the contents of which are herein incorporated by reference in their entirety)) and a database that consisted of common lab contaminants. The MS mass tolerance was set at +10 ppm and MS/MS mass tolerance was set at +0.4 Da for the proteome. Protein tandem mass tag (TMTpro) on C- and N-terminus of peptides and carbamidomethyl on cysteine (CAM) were set as static modifications. Methionine oxidation, protein N-terminal acetylation, protein N-terminal methionine loss, or protein N-terminal methionine loss plus acetylation were set as dynamic modifications for proteome data. Peptide-identification algorithm Percolator (Käll et al., 2007 and The et al., 2016 (the contents of which are herein incorporated by reference in their entirety)) was used for results validation. A concatenated reverse database was used for the target-decoy strategy.

For reporter ion quantification, the reporter abundance was set to use the signal/noise ratio (S/N) only if all spectrum files had S/N values, otherwise, intensities were used instead of S/N values. The ‘quant’ value was corrected for isotopic impurity of reporter ions. The co-isolation threshold was set at 50%. The average reporter S/N threshold was set to 10 and the SPS mass matches percent threshold was set to 65%. The protein abundance of each channel was calculated using summed S/N of all unique and razor peptides. Finally, the abundance was further normalized to a summed abundance value for each channel over all peptides identified within a file. Only peptide sequences from genes predicted in the M. capitata genome were used in the present disclosure. Proteins with a false discovery rate (FDR)<0.01 were considered “high” confidence, and proteins with a FDR≥0.01 but <0.05 were considered “medium” confidence. Differentially expressed proteins (DEPs; fold-change [FC]>1, p-value <0.05) were identified between the ambient and high temperature treatments for each time point using the stats v4.1.2 and mixOmics v6.18.1 R packages (Rohart et al., 2017 (the contents of which are herein incorporated by reference in their entirety)). Adjusted p-values were not used for this analysis due to the low number of replicates per condition.

### Transcriptomic Data Method

RNA-seq data was generated for M. capitata genotype MC-289 across the three analyzed time points, two treatment conditions, and field colony (n=3 replicates each). The methods for cDNA library preparation, sequencing, and data analysis are detailed in Williams et al., 2021b (the contents of which are herein incorporated by reference in their entirety). Briefly, a Qiagen ALLPREP® DNA/RNA/miRNA Universal Kit (Qiagen, Germantown, MD, USA) was used to extract RNA from the (crushed) frozen samples; a TRUSEQ® RNA Sample Preparation Kit v2 (Illumina, San Diego, CA, USA) was used to generate strand specific cDNA libraries that were sequenced on a NOVASEQ® machine (Illumina) (2×150 bp flow cell). This protocol included a poly-A selection step, which enriched for transcripts from eukaryotic cells and depleted those from the prokaryotic microbiome. RNA-seq reads were trimmed for low quality bases and adapters using Trimmomatic v0.38 (Bolger et al., 2014 (the contents of which are herein incorporated by reference in their entirety)); read pairs where both mates survived trimming were used to quantify the expression levels of the genes predicted in the M. capitata genome (Shumaker et al., 2019 (the contents of which are herein incorporated by reference in their entirety)) using Salmon v1.10 (Patro et al., 2017 (the contents of which are herein incorporated by reference in their entirety)). Differentially expressed genes (DEGs; FC >1, adjusted p-value <0.05) were identified between the ambient and high temperature treatments at each time point by the DESeq2 v1.34.0 R package (Love et al., 2014 (the contents of which are herein incorporated by reference in their entirety)) using the aligned read counts produced by Salmon. The Transcripts Per Million (TPM) normalized expression values produced by Salmon were used for all downstream visualization and ordination analyses. Transcripts with a cumulative TPM>100 (i.e., >100 TPM summed across all samples) were used for the ordination and statistical analysis.

### Gene Functional Annotation

Functional assignment of the M. capitata proteins was done using functional annotation tool eggNOG-mapper (v2.1.6; --pfam_realign denovo; database release 2021 Dec. 9) (Cantalapiedra et al., 2021; Huerta-Cepas et al., 2019 (the contents of which are herein incorporated by reference in their entirety)) and a DIAMOND protein aligner search (v2.0.15; blastp--ultra-sensitive--max-target-seqs 1000) (Buchfink et al., 2021 (the contents of which are herein incorporated by reference in their entirety)) against the National Center for Biotechnology Information (NCBI) non-redundant (nr) database (release 2022_07). eggNOG-mapper was also used to assign KEGG orthologous numbers (KO numbers).

### Proportion of Shared SNPs Between Transcriptome Samples

The proportion of single nucleotide polymorphisms (SNPs) shared between each pairwise combination of transcriptome samples was used to confirm that they were all derived from the same colony (genotype). Each sample was aligned against the M. capitata reference genome (Shumaker et al., 2019 (the contents of which are herein incorporated by reference in their entirety)) using transcript alignment tool STAR (v2.7.8a;--sjdbOverhang 149--twopassMode Basic) (Dobin et al., 2013 (the contents of which are herein incorporated by reference in their entirety)). Read-group information was extracted from the read names using read-group inference tool rgsam (v0.1; github.com/djhshih/rgsam; --qnformat illumina-1.8) and added to the aligned reads using genome analysis toolkit gatk ‘FastqToSam’ and gatk ‘MergeBamAlignment’ (--INCLUDE_SECONDARY_ALIGNMENTS false--VALIDATION_STRINGENCY SILENT). Duplicate reads were removed using gatk ‘MarkDuplicates’ (--VALIDATION_STRINGENCY SILENT) before reads that spanned intron-exon boundaries were split using gatk ‘SplitNCigarReads’ (default). Haplotypes were called using gatk ‘HaplotypeCaller’ (--dont-use-soft-clipped-bases-ERC GVCF), with the resulting genomic variant call format (GVCF) files (one per sample) combined using gatk ‘CombineGVCFs’ before being jointly genotyped using gatk ‘GenotypeGVCFs’ (-stand-call-conf 30) (Poplin et al., 2018 (the contents of which are herein incorporated by reference in their entirety)). The resulting variant were filtered for indels, sites with low average reads coverage across all samples, and sites without called genotypes across all samples using variant call format (VCF) package VCFtools (v0.1.17;--remove-indels--min-meanDP 10--max-missing 1.0) (Danecek et al., 2011 (the contents of which are herein incorporated by reference in their entirety)). The “vcf_clone_detect.py” script (from github.com/pimbongaerts/radseq; retrieved Jun. 12, 2021) was used with the filtered variants to compute the number of SNPs shared between each pair of samples.

### Results

There were 4036 M. capitata proteins which had peptides identified in at least one of the proteomic samples (3882 [96.18%] were high confidence identifications). Of these proteins, 2760 (68.38%) had KEGG orthologous (KO) numbers assigned, with 414 (15%) of these belonging to at least one of the major biochemical pathways presented in Table 1. In comparison, out of the 63,227 predicted proteins in the M. capitata genome, 18,684 (29.55%) have annotated KO numbers and 1925 (10.3%) belonged to at least one major biochemical pathway.

TABLE 1
Major KEGG pathways used to filter proteins
identified in the proteomic data

| Pathway Name | Pathway ID |
|---|---|
| Glycolysis/Gluconeogenesis | ko00010 |
| Citrate cycle (tricarboxylic acid (TCA) cycle) | ko00020 |
| Pentose phosphate pathway | ko00030 |
| Amino sugar and nucleotide sugar metabolism | ko00520 |
| Pyruvate metabolism | ko00620 |
| Oxidative phosphorylation | ko00190 |
| Nitrogen metabolism | ko00910 |
| Fatty acid biosynthesis | ko00061 |
| Steroid hormone biosynthesis | ko00140 |
| Glycerolipid metabolism | ko00561 |
| Glycerophospholipid metabolism | ko00564 |
| Purine metabolism | ko00230 |
| Pyrimidine metabolism | ko00240 |
| Alanine, aspartate, and glutamate metabolism | ko00250 |
| Glycine, serine, and threonine metabolism | ko00260 |
| Cysteine and methionine metabolism | ko00270 |
| Valine, leucine, and isoleucine degradation | ko00280 |
| Valine, leucine, and isoleucine biosynthesis | ko00290 |
| Lysine biosynthesis | ko00300 |
| Lysine degradation | ko00310 |
| Arginine biosynthesis | ko00220 |
| Arginine and proline metabolism | ko00330 |
| Histidine metabolism | ko00340 |
| Tyrosine metabolism | ko00350 |
| Phenylalanine metabolism | ko00360 |
| Tryptophan metabolism | ko00380 |
| Phenylalanine, tyrosine, and tryptophan biosynthesis | ko00400 |

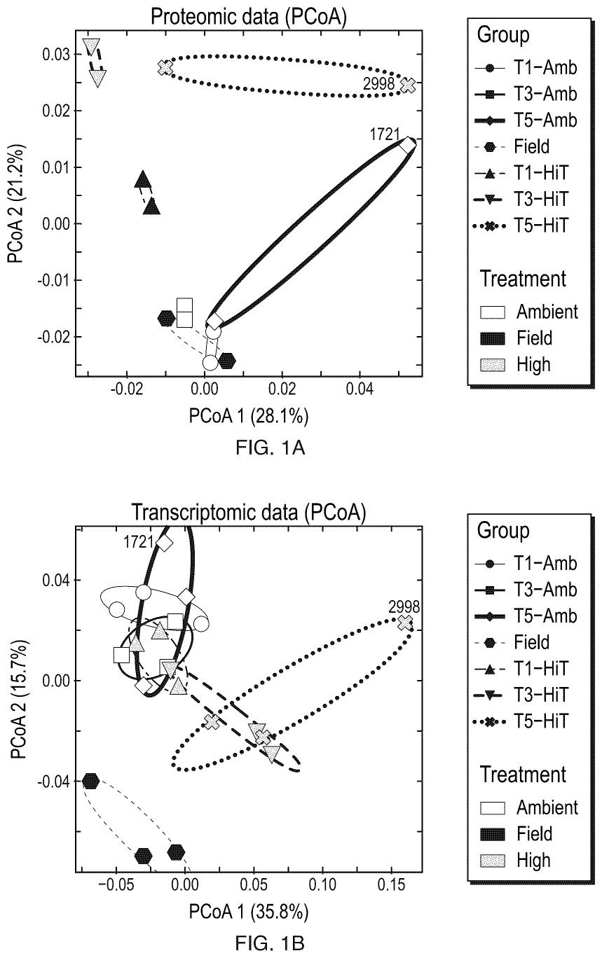

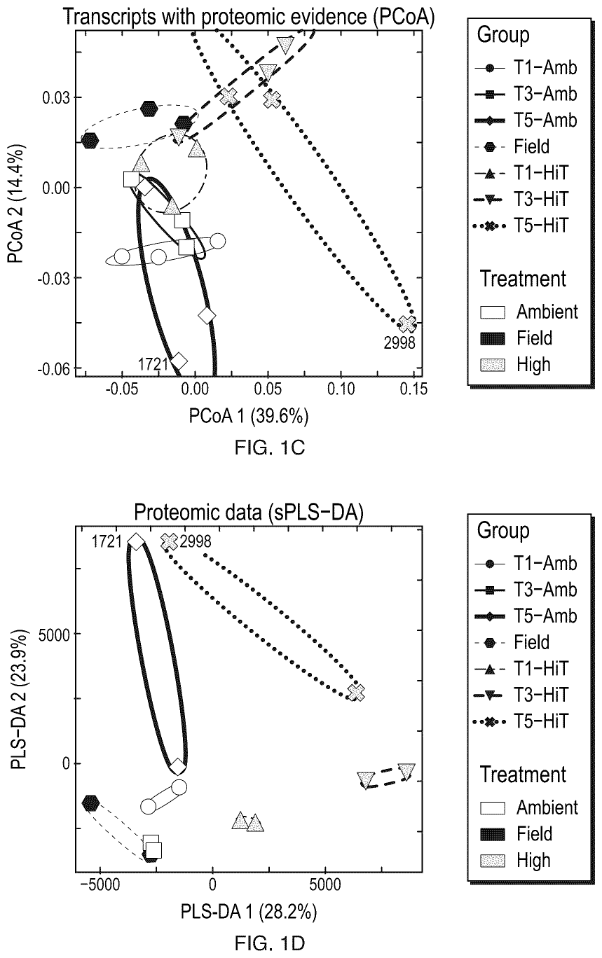

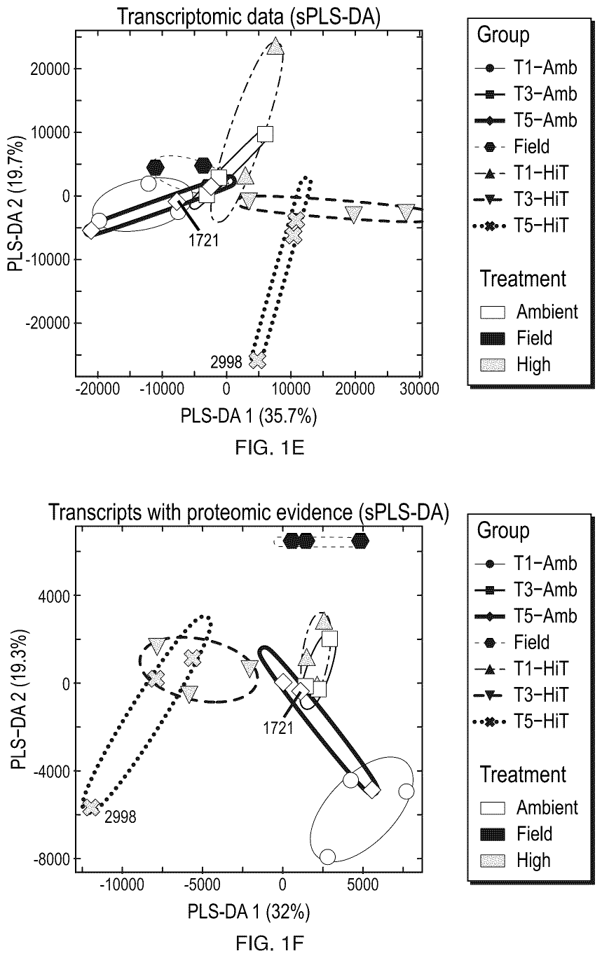

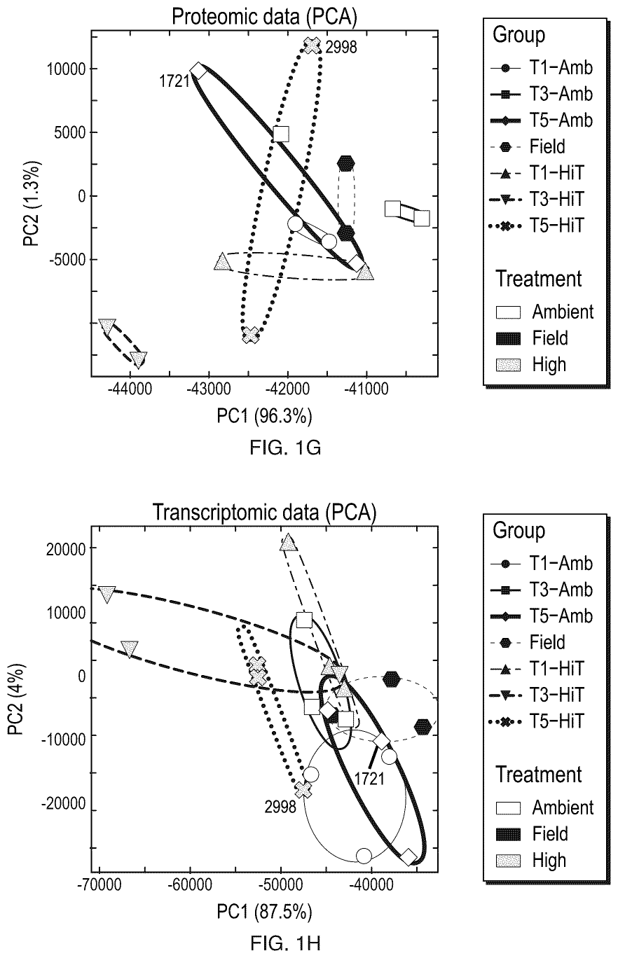

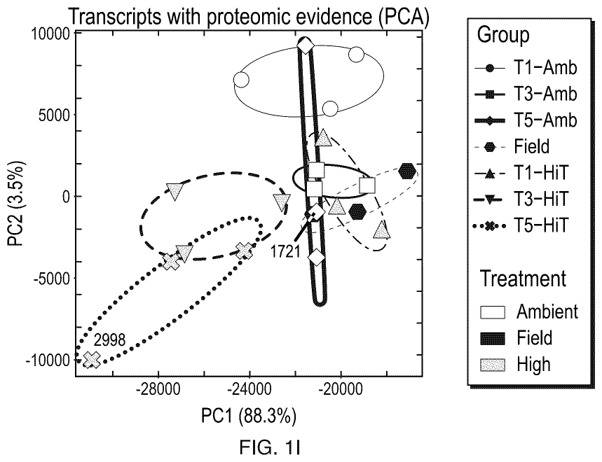

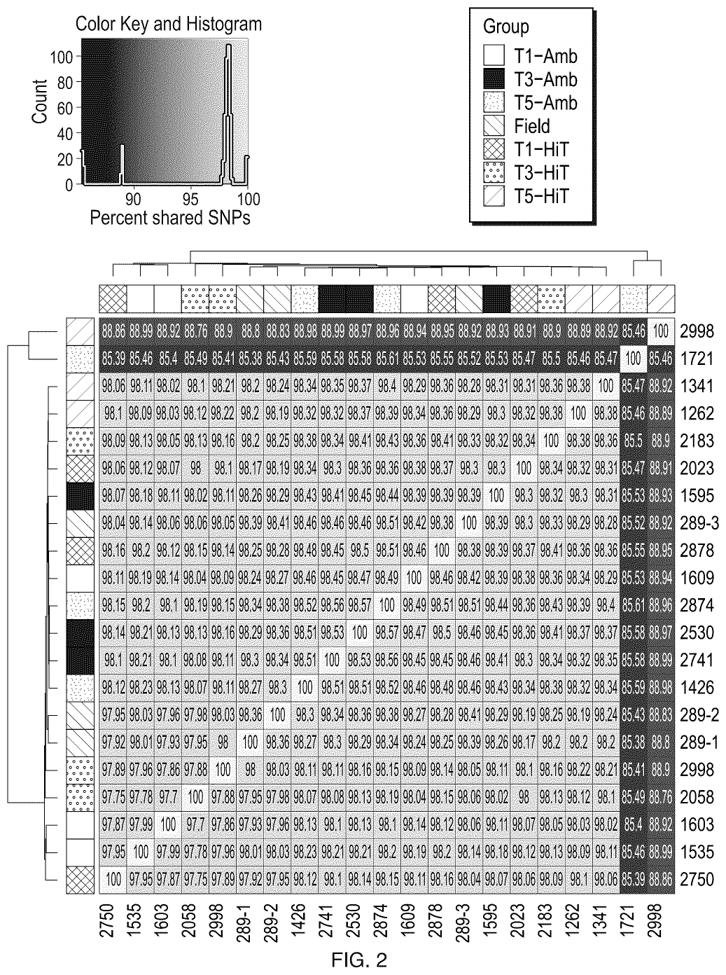

The principal coordinate analysis (PCoA) plots of the proteomic (FIG. 1A) and transcriptomic (FIG. 1B) data from a single M. capitata genotype (MC-289) demonstrate that samples group well by time point and treatment. The field samples tend to group closely with the ambient samples because this was not a period of bleaching in Kãne'ohe Bay, O'ahu where the experiments were carried out. This result is supported by sparse partial least squares-discriminant analysis (sPLS-DA) (FIGS. 1D and 1E) and principal component analysis (PCA) (FIGS. 1G and 1H) plots, which also show the samples in each dataset grouping by time point and treatment. Generally, samples from the same group are positioned close together across the different analysis plots, particularly in the proteomic dataset, although, there are some outliers (i.e., MC-289_T5-HIT_2998 and MC-289_T5-Amb_1721 in FIGS. 1A and 1B). Genotyping showed that all but two (MC-289_T5-HiT_2998 and MC-289_T5-Amb_1721) of the transcriptome samples have high (˜98%) proportions of shared SNPs, confirming that they are all from the same genotype. The two samples share <90% of their SNPs with each other and with the other samples, suggesting that they are from different genotypes (FIG. 2, Table 2). This is potentially due to a mix-up in sample labeling. Inclusion of these samples in downstream analysis is unlikely to greatly affect the interpretations due to the statistical approaches used that take sample variance into account, and because the present disclosure focuses discussion on the samples from time point 1 (TP1 or T1) and time point 3 (TP3 or T3), which are all confirmed to be from the same genotype.

TABLE 2
Proportion of shared SNPs between all pairwise combinations
of the MC-289 (corresponds with FIG. 2 heatmap)

| | | | Data column | | | | | | |
|---|---|---|---|---|---|---|---|---|---|
| | | | 1 | 2 | 3 | 4 | 5 | 6 | 7 |
| | | | Time point | | | | | | |
| | | | T1 | T1 | T1 | T3 | T3 | | |
| | | | Condition | | | | | | |
| | | | HiT | Amb | Amb | HiT | HiT | Field | Field |
| | | | Sample | | | | | | |
| | | | 2750 | 1535 | 1603 | 2058 | 2998 | 289-1 | 289-2 |
| T5 | HiT | 2998 | 88.86 | 88.99 | 88.92 | 88.76 | 88.9 | 88.8 | 88.83 |
| T5 | HiT | 1721 | 85.39 | 85.46 | 85.4 | 85.49 | 85.41 | 85.38 | 85.43 |
| T5 | Amb | 1341 | 98.06 | 98.11 | 98.02 | 98.1 | 98.21 | 98.2 | 98.24 |
| T5 | HiT | 1262 | 98.1 | 98.09 | 98.03 | 98.12 | 98.22 | 98.2 | 98.19 |
| T3 | HiT | 2183 | 98.09 | 98.13 | 98.05 | 98.13 | 98.16 | 98.2 | 98.25 |
| T1 | HiT | 2023 | 98.06 | 98.12 | 98.07 | 98 | 98.1 | 98.17 | 98.19 |
| T3 | Amb | 1595 | 98.07 | 98.18 | 98.11 | 98.02 | 98.11 | 98.26 | 98.29 |
| | Field | 289-3 | 98.04 | 98.14 | 98.06 | 98.06 | 98.05 | 98.39 | 98.41 |
| T1 | HiT | 2878 | 98.16 | 98.2 | 98.12 | 98.15 | 98.14 | 98.25 | 98.28 |
| T1 | Amb | 1609 | 98.11 | 98.19 | 98.14 | 98.04 | 98.09 | 98.24 | 98.27 |
| T5 | Amb | 2874 | 98.15 | 98.2 | 98.1 | 98.19 | 98.15 | 98.34 | 98.38 |
| T3 | Amb | 2530 | 98.14 | 98.21 | 98.13 | 98.13 | 98.16 | 98.29 | 98.36 |
| T3 | Amb | 2741 | 98.1 | 98.21 | 98.1 | 98.08 | 98.11 | 98.3 | 98.34 |
| T5 | Amb | 1426 | 98.12 | 98.23 | 98.13 | 98.07 | 98.11 | 98.27 | 98.3 |
| | Field | 289-2 | 97.95 | 98.03 | 97.96 | 97.98 | 98.03 | 98.36 | 100 |
| | Field | 289-1 | 97.92 | 98.01 | 97.93 | 97.95 | 98 | 100 | 98.36 |
| T3 | HiT | 2998 | 97.89 | 97.96 | 97.86 | 97.88 | 100 | 98 | 98.03 |
| T3 | HiT | 2058 | 97.75 | 97.78 | 97.7 | 100 | 97.88 | 97.95 | 97.98 |
| T1 | Amb | 1603 | 97.87 | 97.99 | 100 | 97.7 | 97.86 | 97.93 | 97.96 |
| T1 | Amb | 1535 | 97.95 | 100 | 97.99 | 97.78 | 97.96 | 98.01 | 98.03 |
| T1 | HiT | 2750 | 100 | 97.95 | 97.87 | 97.75 | 97.89 | 97.92 | 97.95 |
| | | | Data column | | | | | | |
| | | | 8 | 9 | 10 | 11 | 12 | 13 | 14 |
| | | | Time point | | | | | | |
| | | | T5 | T3 | T3 | T5 | T1 | T1 | |
| | | | Condition | | | | | | |
| | | | Amb | Amb | Amb | Amb | Amb | HiT | Field |
| | | | Sample | | | | | | |
| | | | 1426 | 2741 | 2530 | 2874 | 1609 | 2878 | 289-3 |
| T5 | HiT | 2998 | 88.98 | 88.99 | 88.97 | 88.96 | 88.94 | 88.95 | 88.92 |
| T5 | HiT | 1721 | 85.59 | 85.58 | 85.58 | 85.61 | 85.53 | 85.55 | 85.52 |
| T5 | Amb | 1341 | 98.34 | 98.35 | 98.37 | 98.4 | 98.29 | 98.36 | 98.28 |
| T5 | HiT | 1262 | 98.32 | 98.32 | 98.37 | 98.39 | 98.34 | 98.36 | 98.29 |
| T3 | HiT | 2183 | 98.38 | 98.34 | 98.41 | 98.43 | 98.36 | 98.41 | 98.33 |
| T1 | HiT | 2023 | 98.34 | 98.3 | 98.36 | 98.36 | 98.38 | 98.37 | 98.3 |
| T3 | Amb | 1595 | 98.43 | 98.41 | 98.45 | 98.44 | 98.39 | 98.39 | 98.39 |
| | Field | 289-3 | 98.46 | 98.46 | 98.46 | 98.51 | 98.42 | 98.38 | 100 |
| T1 | HiT | 2878 | 98.48 | 98.45 | 98.5 | 98.51 | 98.46 | 100 | 98.38 |
| T1 | Amb | 1609 | 98.46 | 98.45 | 98.47 | 98.49 | 100 | 98.46 | 98.42 |
| T5 | Amb | 2874 | 98.52 | 98.56 | 98.57 | 100 | 98.49 | 98.51 | 98.51 |
| T3 | Amb | 2530 | 98.51 | 98.53 | 100 | 98.57 | 98.47 | 98.5 | 98.46 |
| T3 | Amb | 2741 | 98.51 | 100 | 98.53 | 98.56 | 98.45 | 98.45 | 98.46 |
| T5 | Amb | 1426 | 100 | 98.51 | 98.51 | 98.52 | 98.46 | 98.48 | 98.46 |
| | Field | 289-2 | 98.3 | 98.34 | 98.36 | 98.38 | 98.27 | 98.28 | 98.41 |
| | Field | 289-1 | 98.27 | 98.3 | 98.29 | 98.34 | 98.24 | 98.25 | 98.39 |
| T3 | HiT | 2998 | 98.11 | 98.11 | 98.16 | 98.15 | 98.09 | 98.14 | 98.05 |
| T3 | HiT | 2058 | 98.07 | 98.08 | 98.13 | 98.19 | 98.04 | 98.15 | 98.06 |
| T1 | Amb | 1603 | 98.13 | 98.1 | 98.13 | 98.1 | 98.14 | 98.12 | 98.06 |
| T1 | Amb | 1535 | 98.23 | 98.21 | 98.21 | 98.2 | 98.19 | 98.2 | 98.14 |
| T1 | HiT | 2750 | 98.12 | 98.1 | 98.14 | 98.15 | 98.11 | 98.16 | 98.04 |
| | | | Data column | | | | | | |
| | | | 15 | 16 | 17 | 18 | 19 | 20 | 21 |
| | | | Time point | | | | | | |
| | | | T3 | T1 | T3 | T5 | T5 | T5 | T5 |
| | | | Condition | | | | | | |
| | | | Amb | HiT | HiT | HiT | HiT | Amb | HiT |
| | | | Sample | | | | | | |
| | | | 1595 | 2023 | 2183 | 1262 | 1341 | 1721 | 2998 |
| T5 | HiT | 2998 | 88.93 | 88.91 | 88.9 | 88.89 | 88.92 | 85.46 | 100 |
| T5 | HiT | 1721 | 85.53 | 85.47 | 85.5 | 85.46 | 85.47 | 100 | 85.46 |
| T5 | Amb | 1341 | 98.31 | 98.31 | 98.36 | 98.38 | 100 | 85.47 | 88.92 |
| T5 | HiT | 1262 | 98.3 | 98.32 | 98.38 | 100 | 98.38 | 85.46 | 88.89 |
| T3 | HiT | 2183 | 98.32 | 98.34 | 100 | 98.38 | 98.36 | 85.5 | 88.9 |
| T1 | HiT | 2023 | 98.3 | 100 | 98.34 | 98.32 | 98.31 | 85.47 | 88.91 |
| T3 | Amb | 1595 | 100 | 98.3 | 98.32 | 98.3 | 98.31 | 85.53 | 88.93 |
| | Field | 289-3 | 98.39 | 98.3 | 98.33 | 98.29 | 98.28 | 85.52 | 88.92 |
| T1 | HiT | 2878 | 98.39 | 98.37 | 98.41 | 98.36 | 98.36 | 85.55 | 88.95 |
| T1 | Amb | 1609 | 98.39 | 98.38 | 98.36 | 98.34 | 98.29 | 85.53 | 88.94 |
| T5 | Amb | 2874 | 98.44 | 98.36 | 98.43 | 98.39 | 98.4 | 85.61 | 88.96 |
| T3 | Amb | 2530 | 98.45 | 98.36 | 98.41 | 98.37 | 98.37 | 85.58 | 88.97 |
| T3 | Amb | 2741 | 98.41 | 98.3 | 98.34 | 98.32 | 98.35 | 85.58 | 88.99 |
| T5 | Amb | 1426 | 98.43 | 98.34 | 98.38 | 98.32 | 98.34 | 85.59 | 88.98 |
| | Field | 289-2 | 98.29 | 98.19 | 98.25 | 98.19 | 98.24 | 85.43 | 88.83 |
| | Field | 289-1 | 98.26 | 98.17 | 98.2 | 98.2 | 98.2 | 85.38 | 88.8 |
| T3 | HiT | 2998 | 98.11 | 98.1 | 98.16 | 98.22 | 98.21 | 85.41 | 88.9 |
| T3 | HiT | 2058 | 98.02 | 98 | 98.13 | 98.12 | 98.1 | 85.49 | 88.76 |
| T1 | Amb | 1603 | 98.11 | 98.07 | 98.05 | 98.03 | 98.02 | 85.4 | 88.92 |
| T1 | Amb | 1535 | 98.18 | 98.12 | 98.13 | 98.09 | 98.11 | 85.46 | 88.99 |
| T1 | HiT | 2750 | 98.07 | 98.06 | 98.09 | 98.1 | 98.06 | 85.39 | 88.86 |

When these ordination methods (PCoA, sPLS-DA, and PCA) were applied to only transcripts from genes with proteomic evidence (FIGS. 1C, 1F, and 1I), they all showed that the samples group well by time point and treatment and are roughly congruent with the relative positioning of the samples in the full proteomic and transcriptomic datasets (although the plots are more similar to the latter than the former). For example, in the proteome ordination plots (FIGS. 1A, 1D, and 1G), there is relatively little variation (excluding the mislabeled samples) along each axis between the replicates from each group compared to the variation between groups. In contrast, the transcriptome (FIGS. 1B, 1E, 1H) and transcripts with proteomic evidence (FIGS. 1C, 1F, 1I) ordination plots show greater relative variation between samples from the same group and lower variation between groups, with samples from the same condition (e.g., ambient) often overlapping on both axes. This suggests that whereas the same general stress response pattern is present in both datasets, evidenced by the same relative relationship between the replicates and groups, there are numerous differences. These results provide evidence that the dynamics of the stress response in each-omics data layer differ, even for the same set of genes. The PERMANOVA results (Table 3) show that none of the factors assessed contribute significantly to the variance between groups; however, time point does tend to have the lowest significance across the datasets, which is congruent with the strong association between the field, ambient (all time points), and TP1 high temperature treated (HiT) samples, and the more distant association between the TP3 and time point 5 (TP5) high temperature treated samples in the ordination results. Note that only 190 permutations could be performed with these datasets due to the limited number of samples.

TABLE 3
Results from the PERMANOVA tests run on the transcriptomic,
proteomic, and metabolomic datasets

| Data | Genotype | Variable | R² | F | PR(>F) |
|---|---|---|---|---|---|
| Proteomic | MC-289 | Time point | 0.3226 | 1.9920 | 0.104712042 |
| | | Treatment | 0.1663 | 3.0803 | 0.146596859 |
| | | Time point × | 0.1333 | 1.2350 | 0.151832461 |
| | | Treatment | | | |
| Transcriptomic | | Time point | 0.2886 | 2.8343 | 0.057 |
| | | Treatment | 0.1482 | 4.3658 | 0.104 |
| | | Time point × | 0.0880 | 1.2963 | 0.256 |
| | | Treatment | | | |
| Transcripts with | | Time point | 0.2790 | 2.7330 | 0.06 |
| proteomic evidence | | Treatment | 0.1554 | 4.5661 | 0.112 |
| | | Time point × | 0.0892 | 1.3099 | 0.259 |
| | | Treatment | | | |
| Polar Metabolic | | Time point | 0.3035 | 2.5775 | 0.005* |
| | | Treatment | 0.1159 | 2.9519 | 0.051 |
| | | Time point × | 0.0703 | 0.8949 | 0.708 |
| | | Treatment | | | |
| Polar | all four genotypes | Time point | 0.2991 | 9.4029 | 0.001* |
| Metabolic | (MC-206, MC-248, | Treatment | 0.0123 | 1.5408 | 0.207 |
| | MC-289, MC-291) | Genotype | 0.0804 | 3.3690 | 0.001* |
| | | Time point × | 0.0151 | 0.9476 | 0.52 |
| | | Treatment | | | |
| | | Time point × | 0.0671 | 1.0549 | 0.353 |
| | | Genotype | | | |
| | | Treatment × | 0.0372 | 1.5593 | 0.072 |
| | | Genotype | | | |
| | | Time point × | 0.0434 | 0.9095 | 0.685 |
| | | Treatment × | | | |
| | | Genotype | | | |

*significant result (p-value < 0.05)
R², coefficient of determination; F, F-value; PR(>F), p-value associated with F-statistic

### Protein Biomarkers of Thermal Stress

The protein biomarkers in Table 4 were directly measured in coral samples using proteomics; their presence and concentration can also be inferred using antibody-based assays. The log₂ (fold-change) values are shown when comparing control vs. thermally stressed corals. The last two proteins in the list, GTP-binding nuclear protein Ran and Growth factor receptor bound protein 10 (GRB10), are controls to assess the methods being used and are not stress markers.

TABLE 4
List of stress-related proteins for assessing coral health.

| Type | Proteins | Log₂(fold-change) |
|---|---|---|
| Stress | Phenylalanine-4- | 3.4 |
| biomarker | hydroxylase | |
| Stress | glycine N- | 2.33 |
| biomarker | methyltransferase | |
| Stress | glyoxylate/hydroxypyruvate | 1.6 |
| biomarker | reductase | |
| Stress | 2′-5′-oligoadenylate | 1.5 |
| biomarker | synthetase | |
| Stress | 4-hydroxyphenylpyruvate | 1.46 |
| biomarker | dioxygenase | |
| Stress | Homogentisate 1,2- | 1.29 |
| biomarker | dioxygenase | |
| Control | GTP-binding nuclear | 0.24 |
| | protein Ran | |
| Control | Growth factor receptor | −0.58 |
| | bound protein 10 (GRB10) | |

### Example 2. Protein Abundance-Transcript Expression Level Correlation

### Correlation Between Gene Expression and Protein Abundance

The correlation between gene expression and protein abundance was assessed using the samples from M. capitata genotype MC-289. Only genes (n=4036) which were detected in the proteome data of at least one sample were used in this analysis. The transcripts per kilobase million (TPM)-normalized gene expression and abundance-normalized protein abundance values were scaled using a log₂ transformation (with an offset of 1 to prevent infinite log values) before being plotted (FIGS. 4A-4N). The magnitude and directionality of the stress response of the genes detected in the proteome data was assessed using the log₂ (fold-change) (log₂FC) values computed during differential transcriptome and proteome expression analysis (FIGS. 3A-3C).

### Results

For genes with proteomic evidence, the log₂ (fold-change) (log₂FC) abundance differences between the protein and associated transcript, between the ambient and high temperature treated samples at each time point, were quantified (FIGS. 3A-3C). Each plot in FIGS. 3A-3C is divided into four quadrants (Q1-Q4), with genes in Q1 having positive protein and transcript log₂FC values, Q2 having positive protein and negative transcript log₂FC values, Q3 having negative protein and transcript log₂FC values, and Q4 having negative protein and positive transcript log₂FC values. A trend line fitted to the data from each time point shows that at time point 1 (TP1) there is little correlation (R²=0.01) between the genes transcriptome and proteome log₂FC values. This correlation increases at time point 3 (TP3) (R²=0.11) and to a lesser extent time point 5 (TP5) (R²=0.04), with there being more variation in the magnitude of log₂FC values at TP3 and TP5 compared to TP1. Additionally, while the genes at TP1 form a roughly circular cloud centered around zero transcript and protein log₂FC, there is a more pronounced spread at TP3, and to a lesser degree TP5, of genes through Q1 and Q3 (i.e., the quadrants where transcript and protein log₂FC directionality correlate). Normalized protein and transcript expression levels of proteins identified in the proteomic data were plotted for each combination of time point and treatment (FIGS. 4A-4N). There was a positive but weak correlation (R²=0.07−0.11) between the normalized protein and transcript expression levels of proteins identified in the proteomic data across all samples, regardless of treatment or time point.

A list of 138 genes associated with thermal stress in corals was compiled and used to further assess the correlation between transcript and protein expression (Cleves et al., 2020a; Kenkel et al., 2011; Palmer & Traylor-Knowles, 2012; Williams et al., 2021b; Zoccola et al., 2017 (the contents of which are herein incorporated by reference in their entirety)). Whereas this gene list is enriched for thermal stress-response genes, many general stress-response genes are also included in the target set. Only 55 of the stress-response genes have significant changes in either transcript or protein expression between the ambient and high temperature treated samples at any of the time points. TP3 and TP5 had more stress-response genes that are differentially expressed in the transcriptome (TP1=5/138; TP3=20/138; TP5=16/138); these time points showed a change in the color score of the coral nubbins (FIG. 5). That is, the high temperature samples showed signs of thermal stress at these time points. TP3 also showed more stress-response genes that are differentially expressed in the proteome (TP1=8/138; TP3=29/138; TP5=6/138). There are only nine stress-response genes that are both a differentially expressed gene (DEG) and differentially expressed protein (DEP), and all of them are at TP3. The present disclosure focuses on the expression profile of genes at TP3, because it was the time point at which the corals showed signs of bleaching both in terms of their physiological and transcriptomic responses (Williams et al., 2021b (the contents of which are herein incorporated by reference in their entirety)), and TP1, because it showed little signs of bleaching and represents are more stable homeostatic state. Additionally, the samples from TP1 and TP3 were confirmed to be from the same genotype, which is not the case for TP5, which likely explains the lack of differentially expressed proteins (DEPs) at this time point. At TP1, the majority of stress-response genes (80/138 [58%]) had transcript and protein log₂FC values that were in the same direction (i.e., both positive or both negative). At TP3, even more of the stress-response genes (92/138 [66.7%]) had transcript and protein log₂FC values that were in the same direction. In the total proteome dataset, at TP1 2013/4036 (49.88%) and at TP3 2250/4036 (55.75%) genes had log₂FC values that were in the same direction.

### Discordance Between Coral Animal Transcript and Protein Abundance

Analyses conducted using a single M. capitata genotype shows that the expression patterns of validated coral animal proteins and transcripts are strongly influenced by the time point and treatment at which the samples were collected (FIGS. 1A-1I). Whereas there are no factors that have significant effects on the proteomic and transcriptomic datasets (Table 3), time point did have the lowest significance values compared to treatment (also observed with the 16S microbiome data). There is a strong association between the field, ambient (all time points), and TP1 high temperature treated samples in the ordination plots (FIGS. 1A-1I). There is relatively little variation between samples from the same treatment group in the proteomic data compared to the transcriptomic data. This increased variation in gene expression at the transcript level could result from environmental and temporal (i.e., treatment) factors acting on the large number of genes detected in this dataset, as well as the dynamic feedback between transcripts and proteins and inherent “noise” in the expression of genes in the genome (Raser & O'Shea, 2005; Struhl, 2007 (the contents of which are herein incorporated by reference in their entirety)). In contrast, environmental factors have a less significant impact on the proteomic data because many of the proteins detected in this dataset are critical for cellular function (supported by their higher rate of KO number assignment) and large changes in their abundance would likely be fatal to the organism. This suggests that transcriptome data capture the corals' immediate response to stress, whereas proteome data capture the longer-term response of the animal and are less impacted by gene expression variation.

Interestingly, when the same approaches (i.e., principal coordinate analysis (PCoA), partial least squares-discriminant analysis (PLS-DA), and PCA) are applied to the data from transcripts with proteomic evidence, the a) relative positioning of the different sample groups and b) level of variation between samples within each group are highly similar to the full transcriptomic data set (FIGS. 1A-1I), though some clear differences are apparent (such as the positioning of the T1-Amb and Field samples in FIG. 1F). This suggests that while the proteomic and transcriptomic datasets are both informative about the coral thermal stress response, they are differently impacted by external factors and have different expression dynamics that lead to a disconnect between the observed stress response of the same gene in both datasets (FIGS. 3A-3C). Furthermore, despite the fact that transcripts and proteins from the same gene exhibit a weak but positive correlation in terms of the magnitude of their accumulation (FIGS. 4A-4N), which is consistent with previous observations (Vogel & Marcotte, 2012 (the contents of which are herein incorporated by reference in their entirety)), the log₂FC of their normalized expression levels between ambient and high temperature conditions for each time point differ significantly (FIGS. 3A-3C). The change in the distribution of genes along both axes over the time points, specifically the spread of genes in Q1 and Q3 at TP3 compared to TP1, suggests that the link between protein and transcript log FC may be stronger under stress, although there are still a significant number of genes with conflicting log₂FC values (i.e., those in Q2 and Q4). That is, when the organism is not under severe stress (i.e., TP1), the system is at homeostasis in both the ambient and high temperature samples with stable rates of protein and transcript degradation and synthesis. Under these conditions, the effects of micro-environmental (i.e., specific to each sample) and expression noise will have greater effects, leading to the weaker correlation between the log₂FC of the two datasets. When the organism is under stress, protein degradation (observed in Aiptasia under thermal stress (Oakley et al., 2017 (the contents of which are herein incorporated by reference in their entirety))) and differential regulation of stress-related genes moves the system out of homeostasis, increasing the differences between the ambient and high temperature treatment groups. The increased differential expression of proteins under these conditions has a corresponding effect in the transcriptome (e.g., transcript expression increases to accommodate increased protein syntheses, which is a result of increased protein degradation), which results in the increased correlation between the log₂FC of the two datasets. This is apparent by the differences between TP1 and TP3, specifically the number of DEPs and differentially expressed genes (DEGs), the log₂FC magnitudes, and the distribution of points in Q1 and Q3. Further, the increase in the number of genes with log₂FC values with shared directionality (i.e., both positive or both negative in the transcriptomic and proteomic datasets) does increase at TP3 (from 52.75% to 59.3%) across all genes with proteomic evidence, with this effect even more pronounced in just the selected stress-response genes (from 58% to 67.4%).

These results demonstrate that, in M. capitata, protein presence and abundance do not necessarily correlate to transcript expression, even for genes shown to be related to thermal stress. However, this effect maybe highly dependent on the conditions under which the organism is exposed, with stress likely to result in stronger correlations. There are many well-described processes that can lead to discordance between the proteome and transcriptome, for example, the shorter half-life of mRNA when compared to the encoded protein, particularly if the mRNA is modified or translationally enhanced (this might explain the genes in Q2). Post-transcriptional regulation (RNA silencing via micro RNA (miRNA), increased transcript turnover, or transcriptional regulators), increased protein turnover (protein degradation, either deliberate or caused by misfolding), and protein buffering could also explain this discordance (and could explain the genes in Q4) (Liu et al., 2016; Vogel & Marcotte, 2012 (the contents of which are herein incorporated by reference in their entirety)). This discordance is well characterized in model organisms (Buccitelli & Selbach, 2020; Cox et al., 2005; Hack, 2004; Koussounadis et al., 2015 (the contents of which are herein incorporated by reference in their entirety)). These results therefore demonstrate the utility of these-omics datasets but underline why both transcript and protein abundance data are needed to gain a more meaningful understanding of coral biology.

The present experimental design does not allow for the exploration of the lag between changes in the expression of a transcript and the corresponding change in protein abundance because the timescale of this study was days to weeks, which is typical of coral stress experiments. To explore this issue, transcript and protein abundances samples would need to be collected multiple times per hour. Regardless, the present disclosure demonstrates that at any given time, transcript abundance cannot be assumed to serve as an accurate proxy for protein abundance. Gene expression patterns can of course be used as biomarkers if they show a strong correlation with stress; however, proteomics or protein-specific assays are required to ascertain the true abundance of proteins. These-omics data layers have well-developed and extensive tools and resources available, further enhancing their usefulness when applied to non-model systems.

### Example 3. Polar Metabolomic Data

### Polar Metabolomic Data Method

Polar metabolite data were generated for each of the four genotypes (MC-206, MC-248, MC-289, MC-291) across the three analyzed time points (T1, T3, T5), two treatment conditions (ambient (Amb) or high (HiT) temperature), and field colonies (n=3 replicates each). The methods used for polar metabolite extraction and analysis are described in Williams et al., 2021a (the contents of which are herein incorporated by reference in their entirety). Briefly, metabolites were extracted from each sample with a 40:40:20 (v/v/v) extraction buffer (MeOH: acetonitrile:H₂O+Formic Acid) and followed a protocol optimized for the extraction of water-soluble polar metabolites; the resulting metabolite extracts were separated into phases using hydrophilic interaction liquid chromatography (performed on a Vanquish Horizon ultra-high performance liquid chromatography (UHPLC) system, Thermo Scientific, Waltham, MA, USA). A Thermo Fisher Scientific Q EXACTIVE® Plus mass spectrometer was used for the full-scan MS analysis and to generate the MS² spectra. The resulting metabolite data was analyzed using LS-MS data-processing tool ElMaven (Agrawal et al., 2019 (the contents of which are herein incorporated by reference in their entirety)). Peaks that had ion counts above 50,000 (before normalization) were retained. The metabolite profiles for all samples were aligned using Ordered Bijective Interpolated Warping (OBI-Warp) (Prince & Marcotte 2006 (the contents of which are herein incorporated by reference in their entirety)). Metabolites in the resulting list were filtered, retaining only those with 48 good peaks and a ‘maxQuality’ score of 0.8. The metabolite intensities were normalized using the frozen weights of each sample. These filtered and normalized peaks were used for downstream analysis and to generate total metabolite counts.

### Dipeptide Quantification

Dipeptide metabolite calls made previously using MS¹ full scans from coral samples were verified using parallel reaction monitoring (PRM) second stage mass spectrum (MS²) spectral patterns from the same samples. These spectra were then compared to the high quality MS² spectra derived from pure chemical standards of the analogous metabolites. The spectral fragments from the samples were then assessed for their correlation to the signal of the parental masses. Fragments with low correlation were removed. The trimmed spectral patterns were normalized to the highest signal intensity and subsequently compared to the spectral pattens of the chemical standards, also normalized, for qualitative relatedness (FIG. 8A). A master mix of the stable isotope labeled dipeptide internal standards (IS) were made at uniform concentrations. The IS were then spiked into the pooled sample so that the concentration in the sample would be as follows: 5 nM, 10 nM, 50 nM, 100 nM, 500 nM, 1 μM, and 5 μM. These samples were run under positive polarity and the signal of the endogenous dipeptide level was compared to that of the labeled IS. The point of intersection of the traces identifies the concentration of the endogenous dipeptide level in the samples (FIGS. 8B-8E).

Using the result of the initial concentration curve of the labeled samples, a master mix of the stable isotope labeled dipeptide internal standards (IS) were remade at a more physiologically relevant concentration (arginine-glutamine (RQ), 500 nM; arginine-alanine (RA) and lysine-glutamine (KQ), 100 nM; arginine-valine (RV), 10 nM). A master mix of all the standards at a 10× higher concentration than desired in the samples was prepared and then diluted 1:10 in the sample vial. Quantification experiments included an ambient temperature control at the same time point (8 weeks) and corresponding samples of the Pocillopora acuta (P. acuta) species.

### Results

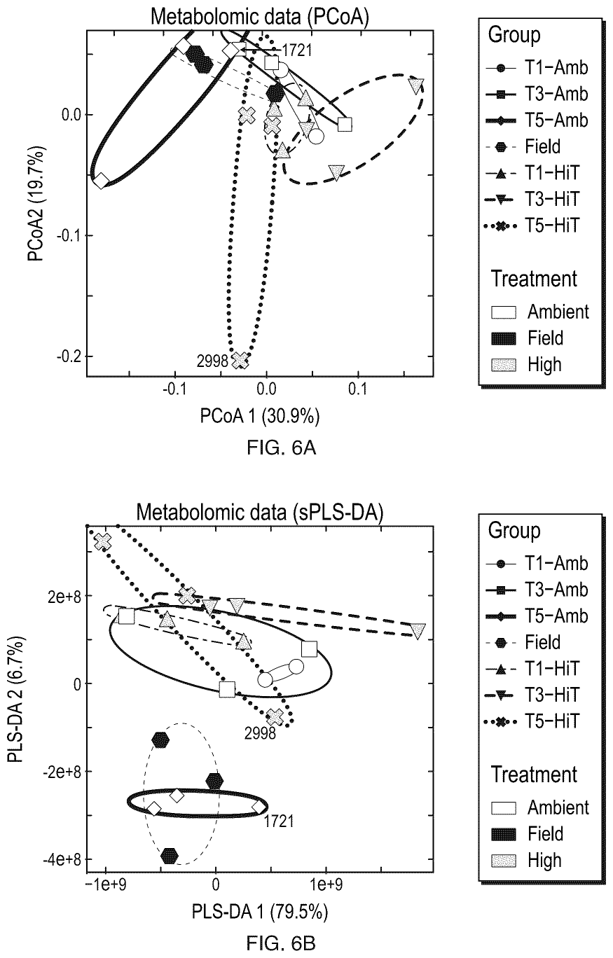

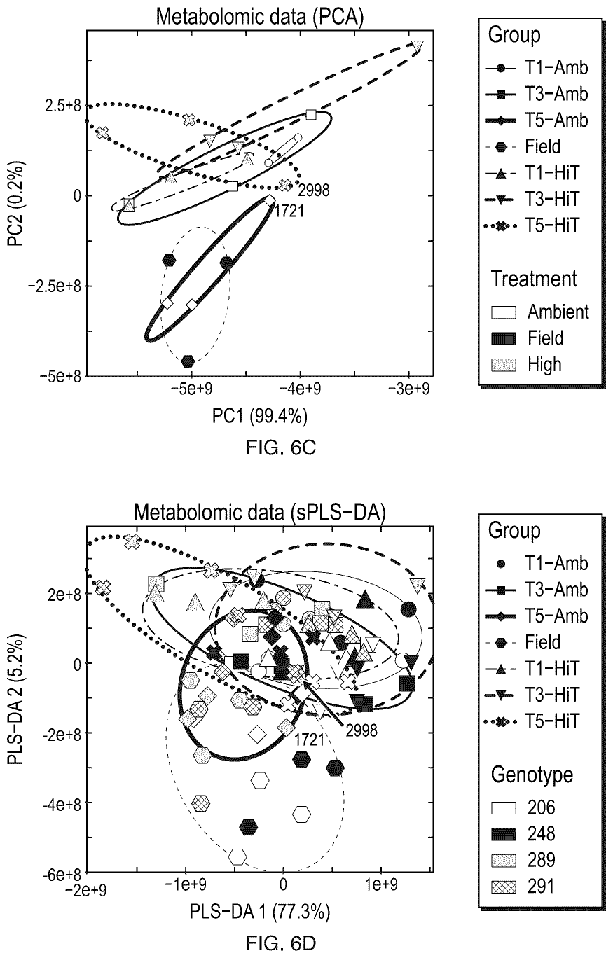

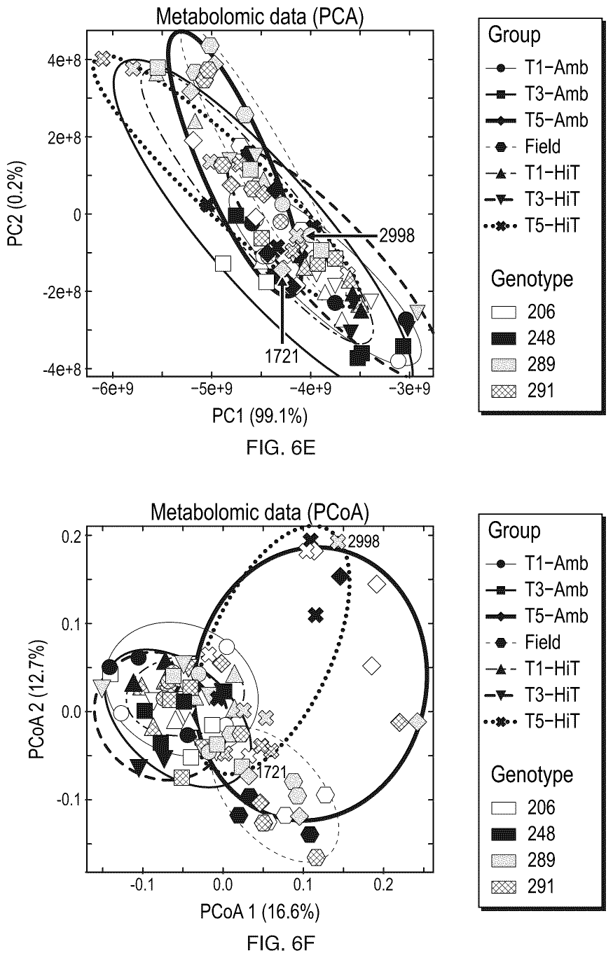

The polar metabolomic dataset generated using positive ionization contained 12,055 peak features. Both supervised (sparse partial least squares-discriminant analysis (sPLS-DA); FIG. 6B) and unsupervised (principal coordinate analysis (PCoA) and principal component analysis (PCA); FIGS. 6A and 6C, respectively) methods, when applied to the filtered metabolite features for a single M. capitata genotype (MC-289) show that samples group by time point and treatment, but without the clear separation (driven by a given factor) between groups observed in the proteomic and transcriptomic ordination plots (i.e., the groups largely overlap; FIGS. 1A-1I). Combining the metabolomic data from all four genotypes (MC-206, MC-248, MC-289, MC-291) into a single analysis does not change this result (FIGS. 6D-6F), with no separation observed between samples from the different genotypes. The permutational multivariate analysis of variance (PERMANOVA) results show that, like for the transcriptomic and proteomic datasets, time point contributed significantly (p-value <0.05) to variation in the metabolomic data, with treatment almost reaching significance. Genotype is a significant factor when data from all four M. capitata genotypes are analyzed; however, none of the interaction terms involving genotype or time point were significant (Table 3).

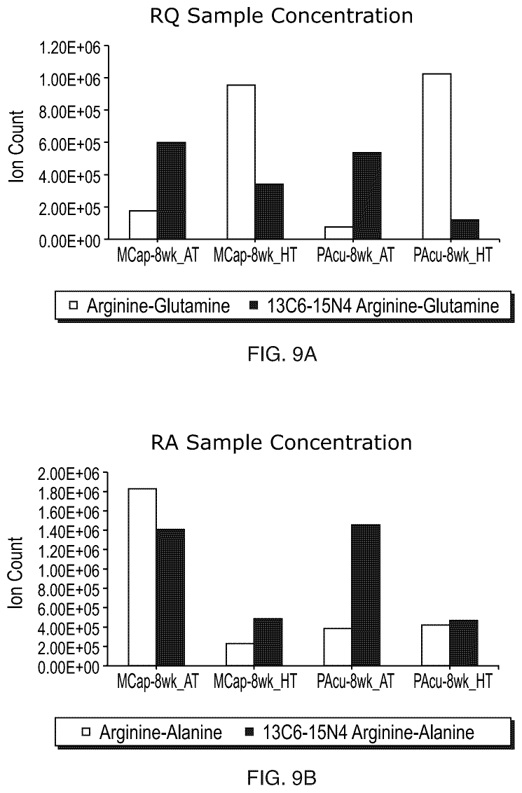

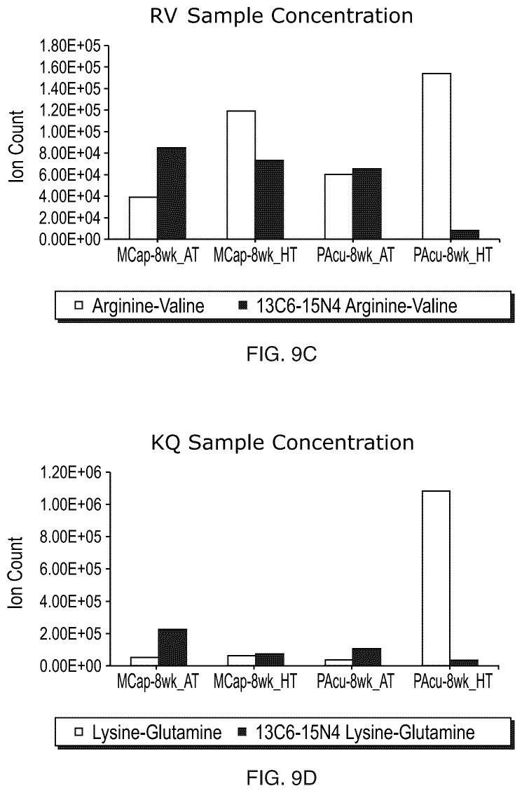

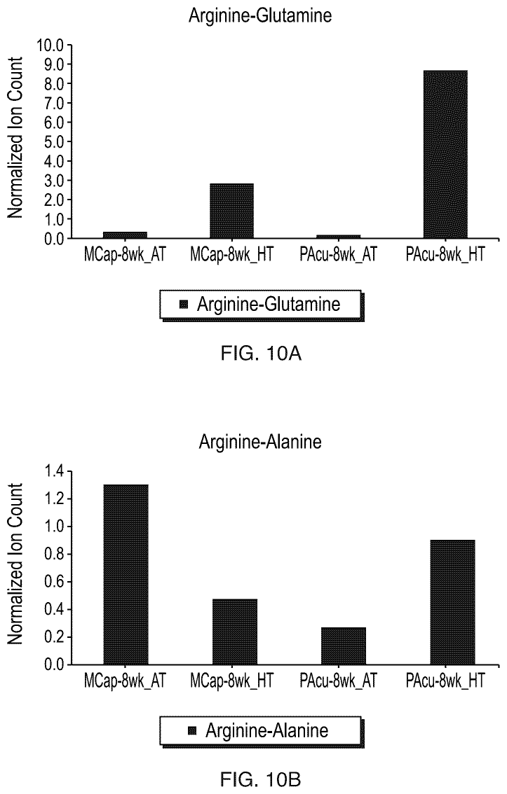

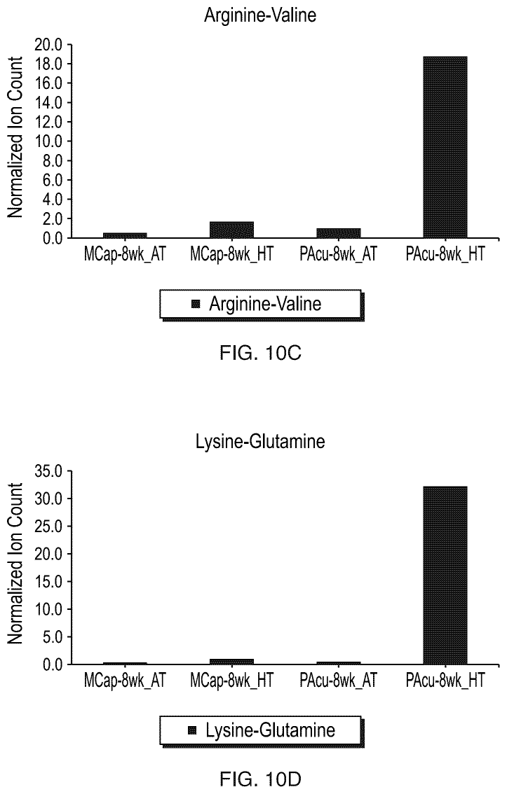

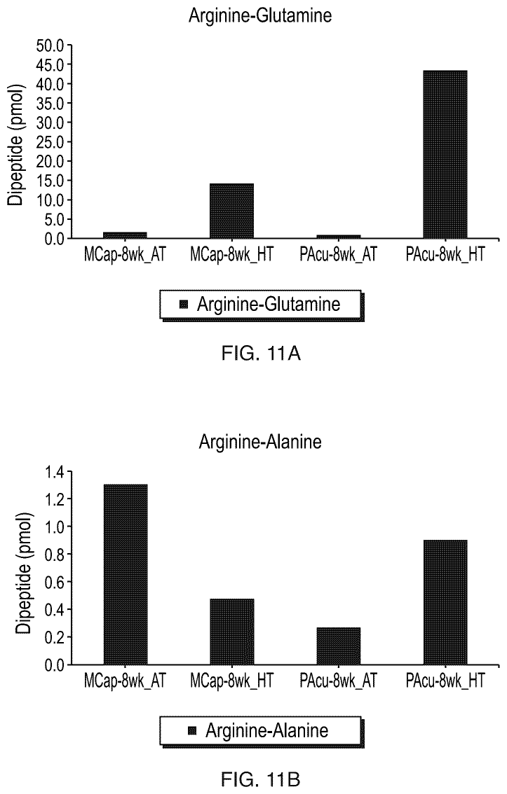

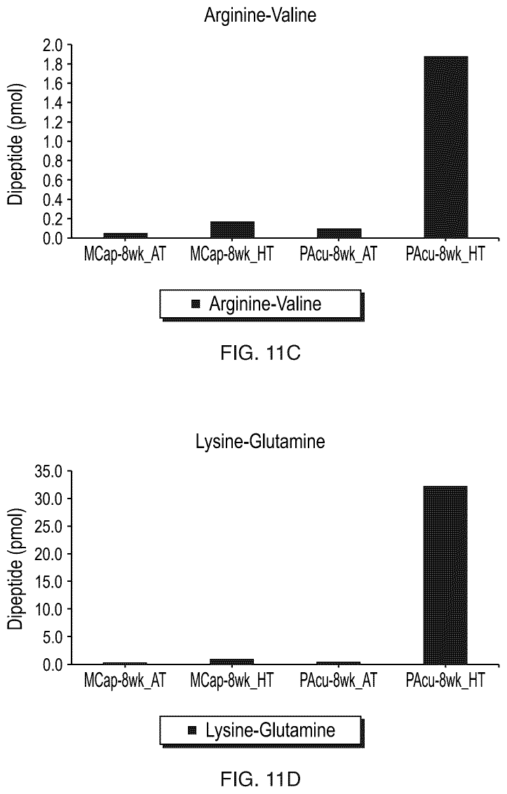

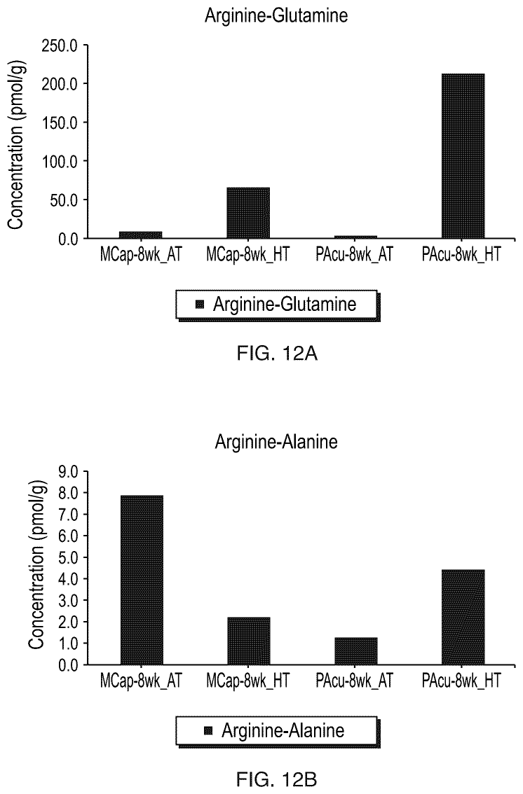

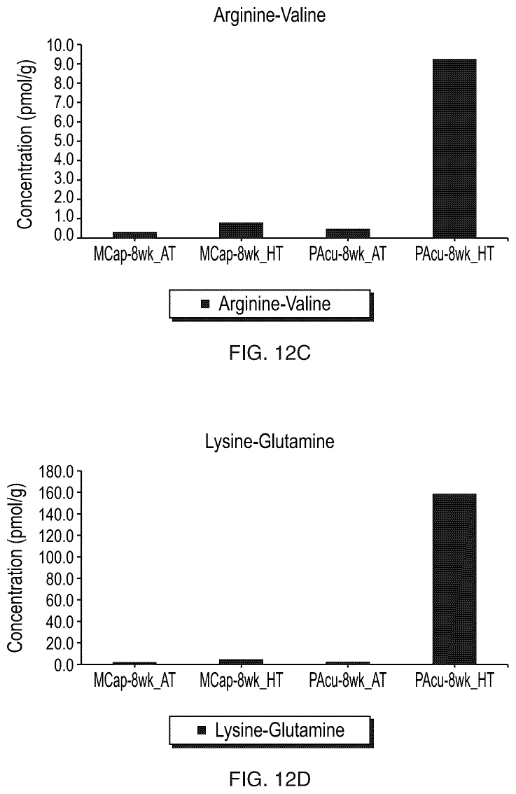

The congruence between the sample and labeled standard trace in FIG. 8A demonstrate that initial identifications using MS¹ full scan were accurate, including for the dipeptides RQ, RA, RV, and KQ (spectra not shown). The standard curve in FIGS. 8B-8E demonstrate that isotopically labeled dipeptides can be used to quantify the absolute concentration (FIGS. 9A-12D) of dipeptides in samples derived from corals.

### Polar Metabolite Levels are Affected by Multiple Experimental Factors

Although the polar metabolomic samples did group by time point and treatment in the supervised (partial least squares-discriminant analysis (PLS-DA)) and unsupervised (PCoA and PCA) ordination plots, the groups often overlap in the single genotype (MC-289) data set (FIGS. 6A-6C), and even more so when combining samples from all four genotypes (MC-206, MC-248, MC-289, MC-291) (FIGS. 6D-6F). These results suggest that total metabolomic data, which includes compounds produced by all members of the coral holobiont, rather than only reflecting the effects of the major factors (i.e., time point and treatment) on the coral host, is significantly influenced by multiple aspects of the holobiont environment, including the complex interactions between the holobiont, host genotype, and the external environment, as well as stochastic and homeostatic processes acting on the holobiont. For example, metabolites such as nucleic acids, organic acids, and organooxygen compounds change very little when corals are exposed to thermal stress (Wilson & Matchinsky, 2021 (the contents of which are herein incorporated by reference in their entirety)). This may be an outcome of metabolic homeostasis in the holobiont, driving the regulation of the levels of these important compounds. In other words, significant changes in the abundance of these metabolites are likely to be deleterious (or even fatal) to the coral, and even under severe stress, their levels may not change significantly. In contrast, previous studies of metabolite data have shown that the accumulation of specific dipeptides (and other metabolites) is significantly correlated with increasing exposure to thermal stress in M. capitata and Pocillopora acuta, regardless of animal genotype (Williams et al., 2021a (the contents of which are herein incorporated by reference in their entirety)). The role of amino acids in stress signaling is well known in animal systems (Hu & Guo, 2020 (the contents of which are herein incorporated by reference in their entirety)).

The present disclosure focuses on small polar molecules which change rapidly in response to metabolic activity and exchange between the organism and its environment (Lu et al., 2017 (the contents of which are herein incorporated by reference in their entirety)). However, other extraction and analysis protocols, which target primary metabolites, such as lipids and fatty acids, can also provide complementary information about the health of the holobiont (particularly given that the algal symbionts may use lipids to transfer energy from photosynthesis to the host (Imbs et al., 2014 (the contents of which are herein incorporated by reference in their entirety))). It should be noted that coral metabolomic data are challenging to interpret for a number of reasons: 1) the presence of many “dark” metabolites limits the utility of untargeted data (Garg, 2021; Williams et al., 2021a (the contents of which are herein incorporated by reference in their entirety)) (i.e., only a few hundred coral metabolites out of tens of thousands, if not hundreds of thousands, that are detected can be identified using available databases (Markley et al., 2017 (the contents of which are herein incorporated by reference in their entirety)); 2) the widely differing metabolite turnover rates necessitates a large number of sample replicates from the same colony to gain statistical significance; and 3) the inability to determine which holobiont component produces each metabolite. Aiptasia may offer an important avenue for addressing some of these problems because it can be maintained in aposymbiotic and symbiotic forms, allowing for the holobiont manipulation required to explore the production and use of specific metabolites.

### Example 4. Microbiome 16S rRNA Data

### Microbiome Data

Microbiome V3-V4 hypervariable region 16S-ribosomal RNA (rRNA) sequencing data were generated for each of the four genotypes (MC-206, MC-248, MC-289, MC-291) across the three analyzed time points (T1, T3, T5), two treatment conditions (ambient (Amb) or high (HiT) temperature), and for the field colonies (n=3 replicates each). The cells in each sample were lysed using liquid nitrogen and mechanical grinding. Total DNA was isolated using a Qiagen ALLPREP® DNA/RNA/miRNA Universal Kit, following the manufacturer's instructions. The 16S-rRNA amplicon sequencing libraries were prepared as per Illumina's instructions (Illumina, 2013), using primers designed for the V3 and V4 hypervariable region, the NEXTERA® XT library preparation kit (Illumina), and dual indexes (i7 and i5). The libraries were pooled together and a 20% PhiX control library spike-in was added. Quality control was performed using a QUBIT® fluorometer (Invitrogen, Waltham, MA, USA) and an Agilent BIOANALYZER®, with the target library length being ˜600 bp. Libraries were sequenced by Genewiz (South Plainfield, NJ, USA) on an Illumina MiSeq machine (2×300 bp flow cell). Raw reads were trimmed for quality and removal of primer sequence using Cutadapt (Martin, 2011). Quality trimming and filtering, denoising, merging and chimera removal, and amplicon sequence variant [ASV] feature table construction were carried out using the QIIME 2 2021.4 plug-in (Bolyen et al., 2019 (the contents of which are herein incorporated by reference in their entirety)) for sample inference tool DADA2 (Callahan et al., 2016 (the contents of which are herein incorporated by reference in their entirety)). Taxonomic assignment was carried out with QIIME2 against the SILVA 16S-rRNA database (release 138) (Quast et al., 2013 (the contents of which are herein incorporated by reference in their entirety)). The initial ASV feature table derived from the trimmed reads was filtered, removing ASV that were: (1) too short (<390 bases); (2) did not have unambiguous taxonomic assignments to at least the phylum-level; (3) had taxonomic assignments of “Archaea”, “Chloroplast” or “Mitochondria”; and (4) had a frequency of <20 reads across all 83 samples. The <20 reads cutoff was chosen based on similar filtering approaches deployed in other studies (Doering et al., 2021 (the contents of which are herein incorporated by reference in their entirety)). These studies, which often consisted of <30 samples, used cutoffs of <10 reads; therefore, to account for the larger number of samples in the analysis, a cutoff of 20 reads was chosen. Per-sample ASV counts were rarefied prior to α- and β-diversity analysis. Shapiro-Wilk tests of Shannon and Simpson α-diversity metrics show that the data are non-normal (p-value <0.01; Table 5). Analyses of α- and β-diversity were carried out in R using high-throughput phylogenetic sequence data analysis package phyloseq (McMurdie & Holmes, 2013 (the contents of which are herein incorporated by reference in their entirety)), statistical functions package stats, and ecology package vegan (Oksanen et al., 2020 (the contents of which are herein incorporated by reference in their entirety)). To visualize β-diversity, samples were rarefied to 39,902 reads per sample (chosen by rarefaction analysis to minimize loss of data) using the ‘rarefy_even_depth’ function in the phyloseq R package (McMurdie & Holmes, 2013 (the contents of which are herein incorporated by reference in their entirety)), after which the Bray-Curtis distances between samples were calculated using the ‘distance’ function.

The psych v2.2.3 R package (Revelle, 2022 (the contents of which are herein incorporated by reference in their entirety)) was used to determine whether significant correlations exist between the 16S-rRNA amplicon and metabolite data. Amplicon count data were agglomerated by taxon at multiple taxonomic ranks—from phylum to genus—for testing using the ‘tax_glom’ function within the phyloseq package. Pairwise correlations using the Spearman method, as well as adjustment of p-values using the Benjamini-Hochberg method, were performed on both raw and normalized (by relative abundance) quantifications using the ‘corr.test’ function. Correlations were retained for further analysis if the associated adjusted p-value was less than 0.05. Furthermore, because Spearman rank correlation analyses are sensitive to low values, only taxa with greater than 200 observations across all samples were considered. Correlations were visualized with correlation plots and histograms, generated using the chart function in the PerformanceAnalytics v2.0.4 R package (Brian G. Peterson et al., 2022 (the contents of which are herein incorporated by reference in their entirety)).

### Results

A total of 12,432 amplicon sequence variants (ASVs) were produced from microbiome 16S rRNA sequencing data. Shapiro-Wilk tests of Shannon and Simpson alpha-diversity (α-diversity, i.e., diversity within a sample) metrics show that the data are non-normal (p-value <0.01; Table 5).

TABLE 5
Results from Shapiro-Wilk normality
test on alpha-diversity metrics
Shapiro-Wilk Normality Test

| alpha-diversity index | p-value |
|---|---|
| Observed | 0.000001232* |
| Shannon | 0.006359* |
| Simpson | 0.00000006574* |

*significant result (p-value < 0.05)

The Kruskal-Wallis rank sum tests (Table 6) and pairwise comparisons using Wilcoxon rank sum tests (Table 7) revealed only time point as having any significant impact on α-diversity metrics (p-values=0.00242 and 0.005086 for Shannon and Simpson metrics, respectively).

TABLE 6
Results from Kruskal-Wallis rank
sum tests on alpha-diversity metrics
Kruskal-Wallis one-way ANOVA

| alpha-diversity index | variable | p-value |
|---|---|---|
| Observed | Genotype | 0.9376 |
| Shannon | Genotype | 0.7342 |
| Simpson | Genotype | 0.738 |
| Observed | Treatment | 0.1283 |
| Shannon | Treatment | 0.08573 |
| Simpson | Treatment | 0.1132 |
| Observed | Time point | 0.1872 |
| Shannon | Time point | 0.00242* |
| Simpson | Time point | 0.005086* |
| Observed | Treatment × Time point | 0.07024 |
| Shannon | Treatment × Time point | 0.02371* |
| Simpson | Treatment × Time point | 0.03133* |

*significant result (p-value < 0.05)

TABLE 7
Results from pairwise Wilcoxon rank sum test on alpha-diversity metrics
Pairwise Wilcoxon rank sum test

| alpha- | | | |
|---|---|---|---|
| diversity index | pair | variable | p-value |
| Observed | MC-206 × MC-248 | Genotype | 0.97 |
| | MC-206 × MC-289 | | 0.97 |
| | MC-206 × MC-291 | | 0.97 |
| | MC-248 × MC-289 | | 0.97 |
| | MC-248 × MC-291 | | 0.97 |
| | MC-289 × MC-291 | | 0.97 |
| Shannon | MC-206 × MC-248 | | 0.88 |
| | MC-206 × MC-289 | | 0.96 |
| | MC-206 × MC-291 | | 0.96 |
| | MC-248 × MC-289 | | 0.96 |
| | MC-248 × MC-291 | | 0.88 |
| | MC-289 × MC-291 | | 0.96 |
| Simpson | MC-206 × MC-248 | | 0.94 |
| | MC-206 × MC-289 | | 0.94 |
| | MC-206 × MC-291 | | 0.94 |
| | MC-248 × MC-289 | | 0.94 |
| | MC-248 × MC-291 | | 0.94 |
| | MC-289 × MC-291 | | 0.94 |
| Observed | Ambient × Field | Treatment | 0.11 |
| | Ambient × High | | 0.82 |
| | Field × High | | 0.11 |
| Shannon | Ambient × Field | | 0.068 |
| | Ambient × High | | 1 |
| | Field × High | | 0.068 |
| Simpson | Ambient × Field | | 0.1 |
| | Ambient × High | | 0.93 |
| | Field × High | | 0.12 |
| Observed | Field × TP1 | Time point | 0.329 |
| | Field × TP3 | | 0.048* |
| | Field × TP5 | | 0.108 |
| | TP3 × TP1 | | 0.048* |
| | TP3 × TP5 | | 0.25 |
| | TP5 × TP1 | | 0.237 |
| Shannon | Field × TP1 | | 0.4 |
| | Field × TP3 | | 0.1 |
| | Field × TP5 | | 0.28 |
| | TP3 × TP1 | | 0.14 |
| | TP3 × TP5 | | 0.28 |
| | TP5 × TP1 | | 0.4 |
| Simpson | Field × TP1 | | 0.383 |
| | Field × TP3 | | 0.015* |
| | Field × TP5 | | 0.122 |
| | TP3 × TP1 | | 0.015* |
| | TP3 × TP5 | | 0.228 |
| | TP5 × TP1 | | 0.122 |
| Observed | TP3-Amb × TP5- | Treatment × | 0.97 |
| | Amb | Time point | |
| | T3-Amb × T1-Amb | | 0.63 |
| | T3-Amb × T3-HiT | | 0.42 |
| | T3-Amb × T5-HiT | | 0.76 |
| | T3-Amb × T1-HiT | | 0.42 |
| | T5-Amb × T1-Amb | | 0.57 |
| | T5-Amb × T5-HiT | | 0.86 |
| | T5-Amb × T1-HiT | | 0.42 |
| | T1-Amb × T1-HiT | | 0.74 |
| | Field × T3-Amb | | 0.29 |
| | Field × T5-Amb | | 0.39 |
| | Field × T1-Amb | | 0.42 |
| | Field × T3-HiT | | 0.2 |
| | Field × T5-HiT | | 0.29 |
| | Field × T1-HiT | | 0.76 |
| | T3-HiT × T5-Amb | | 0.42 |
| | T3-HiT × T1-Amb | | 0.29 |
| | T3-HiT × T5-HiT | | 0.42 |
| | T3-HiT × T1-HiT | | 0.2 |
| | T5-HiT × T1-Amb | | 0.82 |
| | T5-HiT × T1-HiT | | 0.42 |
| Shannon | T3-Amb × T5-Amb | | 0.52 |
| | T3-Amb × T1-Amb | | 0.27 |
| | T3-Amb × T3-HiT | | 0.74 |
| | T3-Amb × T5-HiT | | 0.65 |
| | T3-Amb × T1-HiT | | 0.11 |
| | T5-Amb × T1-Amb | | 0.72 |
| | T5-Amb × T5-HiT | | 0.7 |
| | T5-Amb × T1-HiT | | 0.27 |
| | T1-Amb × T1-HiT | | 0.65 |
| | Field × T3-Amb | | 0.11 |
| | Field × T5-Amb | | 0.27 |
| | Field × T1-Amb | | 0.51 |
| | Field × T3-HiT | | 0.11 |
| | Field × T5-HiT | | 0.18 |
| | Field × T1-HiT | | 0.73 |
| | T3-HiT × T5-Amb | | 0.44 |
| | T3-HiT × T1-Amb | | 0.24 |
| | T3-HiT × T5-HiT | | 0.52 |
| | T3-HiT × T1-HiT | | 0.12 |
| | T5-HiT × T1-Amb | | 0.53 |
| | T5-HiT × T1-HiT | | 0.27 |
| Simpson | T3-Amb × T5-Amb | | 0.554 |
| | T3-Amb × T1-Amb | | 0.277 |
| | T3-Amb × T3-HiT | | 0.976 |
| | T3-Amb × T5-HiT | | 0.554 |
| | T3-Amb × T1-HiT | | 0.073 |
| | T5-Amb × T1-Amb | | 0.554 |
| | T5-Amb × T5-HiT | | 0.666 |
| | T5-Amb × T1-HiT | | 0.368 |
| | T1-Amb × T1-HiT | | 0.66 |
| | Field × T3-Amb | | 0.073 |
| | Field × T5-Amb | | 0.3 |
| | Field × T1-Amb | | 0.554 |
| | Field × T3-HiT | | 0.176 |
| | Field × T5-HiT | | 0.277 |
| | Field × T1-HiT | | 0.729 |
| | T3-HiT × T5-Amb | | 0.554 |
| | T3-HiT × T1-Amb | | 0.3 |
| | T3-HiT × T5-HiT | | 0.66 |
| | T3-HiT × T1-HiT | | 0.176 |
| | T5-HiT × T1-Amb | | 0.549 |
| | T5-HiT × T1-HiT | | 0.3 |

*significant result (p-value < 0.05)

Similarly, when investigating beta-diversity (β-diversity, i.e., diversity between samples), time point was the only significant factor found in analysis of similarity (ANOSIM) tests (Table 8) and the most significant factor in permutational multivariate analysis of variance (PERMANOVA) tests (Table 9) conducted on Bray-Curtis distance matrices (p-value=0.01 and 0.009, respectively).

TABLE 8
Results from analysis of similarity
(ANOSIM) run on beta-diversity metrics
Analysis of Similarity (ANOSIM; 9999 permutations)

| Dissimilarity | Variable | R | Significance |
|---|---|---|---|
| Bray-Curtis | Genotype | 0.01552 | 0.2206 |
| Bray-Curtis | Treatment | 0.0312 | 0.1618 |
| Bray-Curtis | Time point | 0.07033 | 0.01* |

*significant result (p-value < 0.05)

TABLE 9
Results from the statistical tests run on the beta-diversity metric
16S microbiome data PERMANOVA

| Genotype | Variable | R² | F | PR(>F) |
|---|---|---|---|---|
| MC-289 | Time point | 0.27722 | 2.0823 | 0.009* |
| | Treatment | 0.04851 | 1.0932 | 0.045* |
| | Time point × | 0.09736 | 1.097 | 0.32 |
| | Treatment | | | |
| all four genotypes | Time point | 0.06243 | 1.845 | 0.034* |
| (MC-206, MC-248, | Treatment | 0.01422 | 1.2608 | 0.009* |
| MC-289, MC-291) | Genotype | 0.08149 | 2.4082 | 0.001* |
| | Time point × | 0.02736 | 1.2129 | 0.156 |
| | Treatment | | | |
| | Time point × | 0.09001 | 0.8867 | 0.855 |
| | Genotype | | | |
| | Treatment × | 0.03306 | 0.9769 | 0.531 |
| | Genotype | | | |
| | Time point × | 0.08234 | 1.2167 | 0.05 |
| | Treatment × | | | |
| | Genotype | | | |

*significant result (p-value < 0.05)

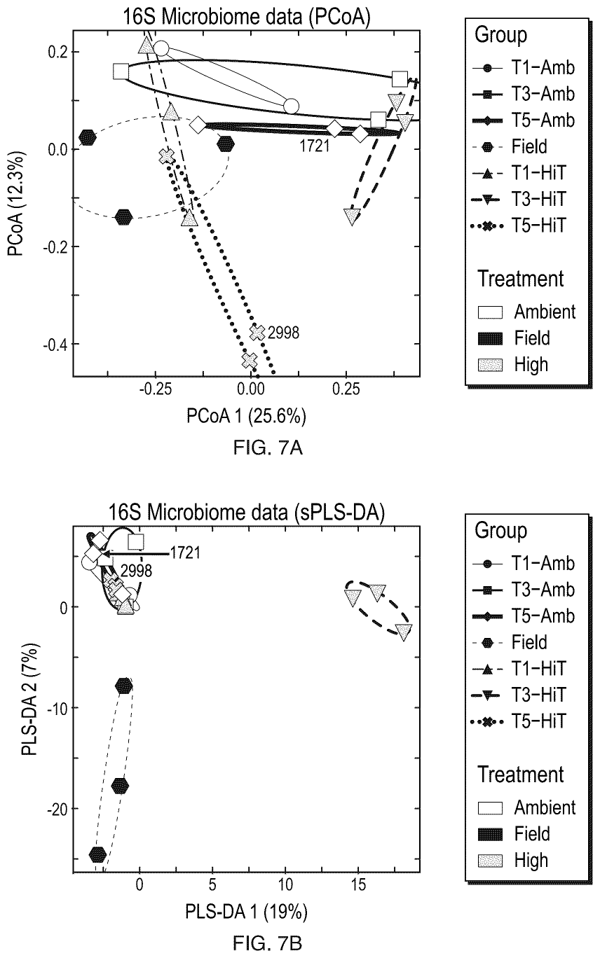

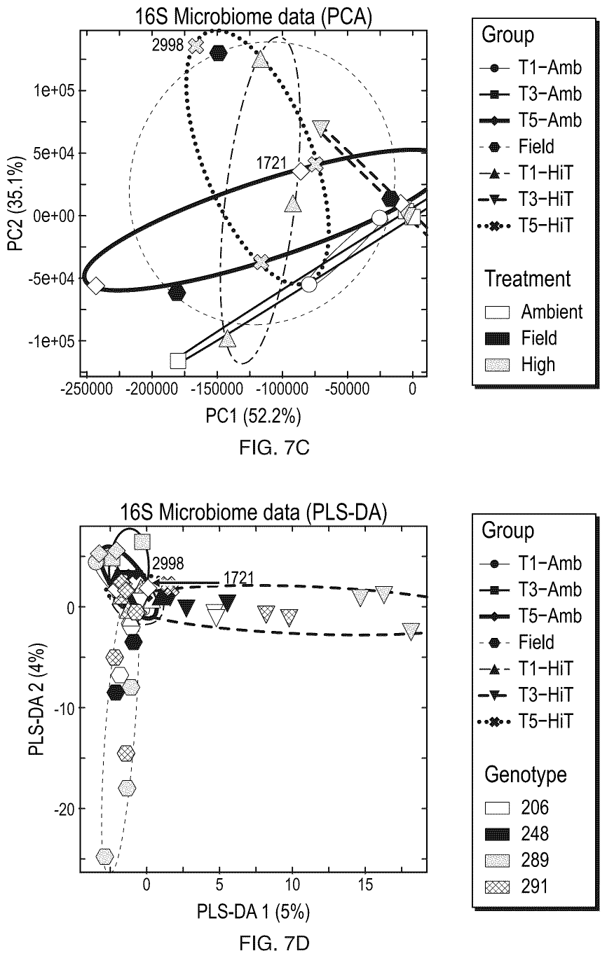

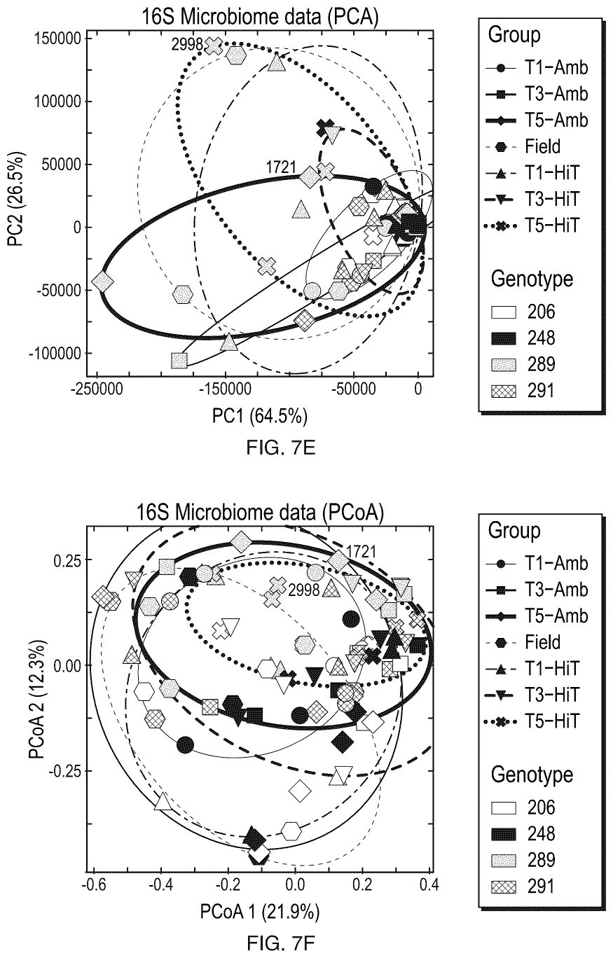

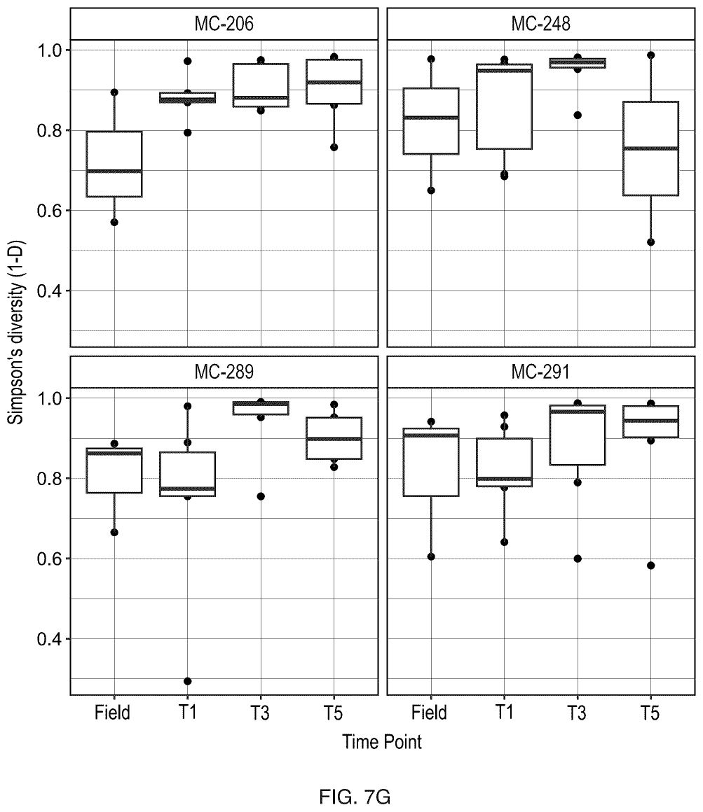

Partial least squares-discriminant analysis (PLS-DA), principal component analysis (PCA), and principal coordinate analysis (PCoA) further demonstrate that bacterial community compositions of the samples are quite dissimilar, even among replicate samples from the same treatment, time point, and genotype (FIGS. 7A-7F). In addition, there is an increase in the a-diversity of the samples from each of the four genotypes over time until TP3 (FIG. 7G). This trend is also reflected in the pairwise Wilcoxon tests between the Field vs. TP3 and TP1 vs. TP3 time points, which have significant (p-value <0.05) associations for the Observed and Simpson's metrics.

### What Are the Factors That Drive Shifts in the Microbiome Profile?

The holobiont microbiome amplicon data show little association with treatment (FIGS. 7A-7G) but do show a significant (p-value <0.05) change in α- and β-diversity metrics over the course of the study (Tables 5-9). This result is likely not explained by a prokaryotic composition that reflects vastly different habitats of origin because these M. capitata colonies were collected from the same reef and the difference in the composition of the genotypes was not statistically significant (Tables 5-9). Therefore, it is likely that change in the microbiome composition of samples over the experiment reflects multiple factors, including gradual acclimation of the samples to the indoor tank environment that used water drawn from Kãne'ohe Bay. In addition, the constant turnover (shedding) of the coral mucus layer every few hours and selection for holobiont fitness may have homogenized the microbiome community in different colonies. Despite significant variability in the composition of the prokaryotic microbiome between different coral species, between species across a broad geographic range, and even across a single coral colony (Botte et al., 2022 (the contents of which are herein incorporated by reference in their entirety)), these prokaryotes undoubtedly play a central role in coral biology and the holobiont response to stress (Hernandez-Agreda et al., 2017; Rosenberg et al., 2007 (the contents of which are herein incorporated by reference in their entirety)).

### Example 5. Correlation Between the Microbiome and the Metabolome Datasets

### Results

At the phylum, class, and order levels, the only significant correlation (r=0.70785488; p-value=5.503×10⁻⁸, 1.276×10⁻⁷, 3.389×10⁻⁷, respectively) that was found is between Marinimicrobia (SAR406 clade) and compound 10951 (m/z=456.11676, rt=1.613358; Table 10). At the family level significant correlations were found again between Marinimicrobia and compound 10951 (r=0.70785488, p-value=5.82×10⁻⁷). Weaker but significant correlations were also found between compound 9802 (m/z=200.961411, rt=5.271) and family LWQ8 (Patescibacteria phylum; r=0.567811104, p-value=0.044185845), as well as between compound 11436 (m/z=596.334717, rt=6.351) and family Coleofasciculaceae (cyanobacteria; r=0.565262299, p-value=0.044185845).

TABLE 10
Table showing Spearman correlation coefficients for those bacterial
families for which significant correlations with metabolites were
found. Adjusted p-values for significant correlations are provided
in bold within parenthesis after the correlation coefficient.

| | groupID | groupID | groupID |
|---|---|---|---|
| Family | 9802 | 10951 | 11436 |
| Coleofasciculaceae | −0.074 | −0.084 | 0.565 (0.044) |
| LWQ8 | 0.568 (0.044) | 0.137 | −0.076 |
| Marinimicrobia | 0.008 | 0.708 | 0.193 |
| (SAR406 clade) | | (5.82 × 10⁻⁷) | |

### The Diversity of Microbial Species Makes it Challenging to Integrate

The algal symbionts were not considered when analyzing both the transcriptomic and proteomic data due to the lack of reference genomes for these diverged taxa and because there are different combinations of species present in the samples, making it challenging to reconstruct the gene inventory from the available RNA-seq data (Stephen et al., 2021 (the contents of which are herein incorporated by reference in their entirety)). Similarly, microbial-associated proteomics data were not included due to the challenges associated with compiling a metaproteomic dataset from reference genomes generated from unrelated environments. Traditional liquid chromatography-tandem mass spectrometry (LC-MS/MS) approaches were established to measure single species with high quality databases, such as reference genomes (Johnson et al., 2020 (the contents of which are herein incorporated by reference in their entirety)). It was too large and highly redundant to create a database of microbial proteins using algal transcripts constructed from the transcriptome data and reference genomes from species closely related to those present in the 16S-rRNA data. Although microbial and symbiont metaproteomic analysis is vital for elucidating holobiont physiological response and ability to adapt to stress, the variability in species composition across samples makes it difficult to develop robust markers of holobiont health. Therefore, the present disclosure advocates for a focus on data that can be unambiguously targeted from the coral to provide a more robust platform for assessing coral stress response and development of markers of coral health.

Correlation analysis between the microbiome and metabolomic data returned very few candidate associations. Given the high variability observed in the microbiome and metabolome, this highlights the challenges associated with integrating these datasets in complex holobiont systems. It is also noteworthy that the identified associations were between metabolites with unknown structures and groups of bacteria that are poorly characterized with only very basic, general characteristics described: Coleofasciculaceae (cyanobacteria) are photosynthetic and may contribute to energy production in the coral holobiont, Patescibacteria form symbiotic associations with other organisms in the environment (Lemos et al., 2019 (the contents of which are herein incorporated by reference in their entirety)), and Marinibacteria are thought to participate in sulfur cycling (Wright et al., 2014 (the contents of which are herein incorporated by reference in their entirety)) and syntrophic degradation of amino acids (Nobu et al., 2015 (the contents of which are herein incorporated by reference in their entirety)). Without knowledge of metabolite structure and function or the ecological role of each bacterial strain identified by this analysis, it is difficult to draw biologically meaningful conclusions from this analysis. This further highlights the challenges and areas where additional resources are required for coral multi-omics analysis. Additionally, given that metabolite levels are affected by the proteins encoded by the bacterial species, and not the species themselves per se, future studies should focus on studying the shifts in bacterial protein inventory between samples rather than taxonomic profiles.

### REFERENCES

Agrawal, S., Kumar, S., Sehgal, R., George, S., Gupta, R., Poddar, S., . . . Pathak, S. (2019). El-MAVEN: A Fast, Robust, and User-Friendly Mass Spectrometry Data Processing Engine for Metabolomics. Methods Mol Biol, 1978, 301-321. Doi: 10.1007/978-1-4939-9236-2_19
Baumgarten, S., Simakov, O., Esherick, L. Y., Liew, Y. J., Lehnert, E. M., Michell, C. T., . . . Voolstra, C. R. (2015). The genome of Aiptasia, a sea anemone model for coral symbiosis. Proc Natl Acad Sci U SA, 112 (38), 11893-11898. Doi: 10.1073/pnas.1513318112
Bhattacharya, D., Agrawal, S., Aranda, M., Baumgarten, S., Belcaid, M., Drake, J. L., . . . Falkowski, P. G. (2016). Comparative genomics explains the evolutionary success of reef-forming corals. Elife, 5. Doi: 10.7554/eLife.13288
Bolger, A. M., Lohse, M., & Usadel, B. (2014). Trimmomatic: A flexible trimmer for Illumina sequence data. Bioinformatics, 30 (15), 2114-2120. Doi: 10.1093/bioinformatics/btu170
Bolyen, E., Rideout, J. R., Dillon, M. R., Bokulich, N. A., Abnet, C. C., Al-Ghalith, G. A., . . . Caporaso, J. G. (2019). Reproducible, interactive, scalable and extensible microbiome data science using QIIME 2. Nat Biotechnol, 37 (8), 852-857. Doi: 10.1038/s41587-019-0209-9
Botte, E. S., Cantin, N. E., Mocellin, V. J. L., O'Brien, P. A., Rocker, M. M., Frade, P. R., & Webster, N. S. (2022). Reef location has a greater impact than coral bleaching severity on the microbiome of Pocillopora acuta. Coral Reefs, 41 (1), 63-79. Doi: 10.1007/s00338-021-02201-y
Brian G. Peterson, Peter Carl, Kris Boudt, Ross Bennett, Joshua Ulrich, Eric Zivot, . . . Shea, J. M. (2022). PerformanceAnalytics: Econometric tools for performance and risk analysis.
Buccitelli, C., & Selbach, M. (2020). mRNAs, proteins and the emerging principles of gene expression control. Nature Reviews Genetics, 21 (10), 630-644. Doi: 10.1038/s41576-020-0258-4
Buchfink, B., Reuter, K., & Drost, H. G. (2021). Sensitive protein alignments at tree-of-life scale using DIAMOND. Nature Methods, 18 (4), 366-+. Doi: 10.1038/s41592-021-01101-x
Callahan, B. J., McMurdie, P. J., Rosen, M. J., Han, A. W., Johnson, A. J., & Holmes,
S. P. (2016). DADA2: High-resolution sample inference from Illumina amplicon data. Nat Methods, 13 (7), 581-583. Doi: 10.1038/nmeth.3869
Cantalapiedra, C. P., Hernandez-Plaza, A., Letunic, I., Bork, P., & Huerta-Cepas, J. (2021). eggnog-mapper v2: Functional annotation, orthology assignments, and domain prediction at the metagenomic scale. Mol Biol Evol, 38 (12), 5825-5829. Doi: 10.1093/molbev/msab293
Cheung, M. W. M., Hock, K., Skirving, W., & Mumby, P. J. (2021). Cumulative bleaching undermines systemic resilience of the Great Barrier Reef. Curr Biol. Doi: 10.1016/j.cub.2021.09.078
Cleves, P. A., Krediet, C. J., Lehnert, E. M., Onishi, M., & Pringle, J. R. (2020a). Insights into coral bleaching under heat stress from analysis of gene expression in a sea anemone model system. Proceedings of the National Academy of Sciences of the United States of America, 117 (46), 28906-28917. Doi: 10.1073/pnas.2015737117
Cleves, P. A., Shumaker, A., Lee, J., Putnam, H. M., & Bhattacharya, D. (2020b). Unknown to Known: Advancing Knowledge of Coral Gene Function. Trends Genet, 36 (2), 93-104. Doi: 10.1016/j.tig.2019.11.001
Costa, R. M., Cardenas, A., Loussert-Fonta, C., Toullec, G., Meibom, A., & Voolstra, C. R. (2021). Surface topography, bacterial carrying capacity, and the prospect of microbiome manipulation in the sea anemone coral model Aiptasia. Frontiers in Microbiology, 12. Doi: 10.3389/fmicb.2021.637834
Cox, B., Kislinger, T., & Emili, A. (2005). Integrating gene and protein expression data: pattern analysis and profile mining. Methods, 35 (3), 303-314. Doi: 10.1016/j.ymeth.2004.08.021
Danecek, P., Auton, A., Abecasis, G., Albers, C. A., Banks, E., DePristo, M. A., . . . Genomes Project Analysis, G. (2011). The variant call format and VCFtools. Bioinformatics, 27 (15), 2156-2158. Doi: 10.1093/bioinformatics/btr330
Dobin, A., Davis, C. A., Schlesinger, F., Drenkow, J., Zaleski, C., Jha, S., . . . Gingeras, T. R. (2013). STAR: Ultrafast universal RNA-seq aligner. Bioinformatics, 29 (1), 15-21. Doi: 10.1093/bioinformatics/bts635
Doering, T., Wall, M., Putchim, L., Rattanawongwan, T., Schroeder, R., Hentschel, U., & Roik, A. (2021). Towards enhancing coral heat tolerance: a “microbiome transplantation” treatment using inoculations of homogenized coral tissues. Microbiome, 9 (1), 102. Doi: 10.1186/s40168-021-01053-6
Garg, N. (2021). Metabolomics in functional interrogation of individual holobiont members. mSystems, 6 (4), e0084121. Doi: 10.1128/mSystems.00841-21
Grottoli, A. G., Toonen, R. J., van Woesik, R., Thurber, R. V., Warner, M. E., McLachlan, R. H., . . . Wu, H. C. (2021). Increasing comparability among coral bleaching experiments. Ecological Applications, 31 (4). Doi: 10.1002/eap.2262
Hack, C. J. (2004). Integrated transcriptome and proteome data: the challenges ahead. Brief Funct Genomic Proteomic, 3 (3), 212-219. Doi: 10.1093/bfgp/3.3.212
Hernandez-Agreda, A., Gates, R. D., & Ainsworth, T. D. (2017). Defining the core microbiome in corals' microbial soup. Trends Microbiol, 25 (2), 125-140. Doi: 10.1016/j.tim.2016.11.003
Hu, X., & Guo, F. (2020). Amino acid sensing in metabolic homeostasis and health. Endocr Rev. doi: 10.1210/endrev/bnaa026
Huerta-Cepas, J., Szklarczyk, D., Heller, D., Hernandez-Plaza, A., Forslund, S. K., Cook, H., . . . Bork, P. (2019). eggnog 5.0: a hierarchical, functionally and phylogenetically annotated orthology resource based on 5090 organisms and 2502 viruses. Nucleic Acids Res, 47 (D1), D309-D314. Doi: 10.1093/nar/gkyl085
Illumina. (2013). Amplicon, P.C.R., Clean-Up, P.C.R. and Index, P. C. R., 2013. 16s metagenomic sequencing library preparation (Part #15044223 Rev. B).
Imbs, A. B., Yakovleva, I. M., Dautova, T. N., Bui, L. H., & Jones, P. (2014). Diversity of fatty acid composition of symbiotic dinoflagellates in corals: Evidence for the transfer of host PUFAs to the symbionts. Phytochemistry, 101, 76-82. Doi: 10.1016/j.phytochem.2014.02.012
Jiang, L., Yoshida, T., Stiegert, S., Jing, Y., Alseekh, S., Lenhard, M., . . . Fernie, A.
R. (2021). Multi-omics approach reveals the contribution of KLU to leaf longevity and drought tolerance. Plant Physiol, 185 (2), 352-368. Doi: 10.1093/plphys/kiaa034
Johnson, R. S., Searle, B. C., Nunn, B. L., Gilmore, J. M., Phillips, M., Amemiya, C. T., . . . MacCoss, M. J. (2020). Assessing protein sequence database suitability using De Novo sequencing. Molecular & Cellular Proteomics, 19 (1), 198-208. Doi: 10.1074/mcp.TIR119.001752
Jokiel, P. L., & Coles, S. L. (1990). Response of Hawaiian and Other Indo-Pacific Reef Corals to Elevated-Temperature. Coral Reefs, 8 (4), 155-162. Doi: Doi 10.1007/Bf00265006
Käll, L., Canterbury, J. D., Weston, J., Noble, W. S., MacCoss, M. J. (2007). Semi-supervised learning for peptide identification from shotgun proteomics datasets. Nat Methods, 4 (11), 923-5. Doi: 10.1038/nmeth1113
Kanehisa, M., & Goto, S. (2000). KEGG: kyoto encyclopedia of genes and genomes. Nucleic Acids Res, 28 (1), 27-30. Doi: 10.1093/nar/28.1.27
Kaplan, M. (2009). Coral may live for thousands of years. Nature. Doi: 10.1038/news.2009.185
Keller-Costa, T., Lago-Leston, A., Saraiva, J. P., Toscan, R., Silva, S. G., Goncalves, J., . . . Costa, R. (2021). Metagenomic insights into the taxonomy, function, and dysbiosis of prokaryotic communities in octocorals. Microbiome, 9 (1), 72. Doi: 10.1186/s40168-021-01031-y
Kenkel, C. D., Aglyamova, G., Alamaru, A., Bhagooli, R., Capper, R., Cunning, R., . . . Matz, M. V. (2011). Development of gene expression markers of acute heat-light stress in reef-building corals of the genus Porites. PloS One, 6 (10). Doi: 10.1371/journal.pone.0026914
Koussounadis, A., Langdon, S. P., Um, I. H., Harrison, D. J., & Smith, V. A. (2015). Relationship between differentially expressed mRNA and mRNA-protein correlations in a xenograft model system. Sci Rep, 5, 10775. Doi: 10.1038/srep10775
Lemos, L. N., Medeiros, J. D., Dini-Andreote, F., Fernandes, G. R., Varani, A. M., Oliveira, G., & Pylro, V. S. (2019). Genomic signatures and co-occurrence patterns of the ultra-small Saccharimonadia (phylum CPR/Patescibacteria) suggest a symbiotic lifestyle. Molecular Ecology, 28 (18), 4259-4271. Doi: 10.1111/mec. 15208
Liu, Y. S., Beyer, A., & Aebersold, R. (2016). On the dependency of cellular protein levels on mRNA abundance. Cell, 165 (3), 535-550. Doi: 10.1016/j.cell.2016.03.014
Love, M. I., Huber, W., & Anders, S. (2014). Moderated estimation of fold change and dispersion for RNA-seq data with DESeq2. Genome Biol, 15 (12), 550. Doi: 10.1186/s13059-014-0550-8
Lu, W. Y., Su, X. Y., Klein, M. S., Lewis, I. A., Fiehn, O., & Rabinowitz, J. D. (2017). Metabolite measurement: Pitfalls to avoid and practices to follow. Annual Review of Biochemistry, Vol 86, 86, 277-304. Doi: 10.1146/annurev-biochem-061516-044952
Markley, J. L., Bruschweiler, R., Edison, A. S., Eghbalnia, H. R., Powers, R., Raftery, D., & Wishart, D. S. (2017). The future of NMR-based metabolomics. Curr Opin Biotechnol, 43, 34-40. Doi: 10.1016/j.copbio.2016.08.001
Martin, M. (2011). Cutadapt removes adapter sequences from high-throughput sequencing reads. EMBnet.journal, 17 (1), 3. Doi: 10.14806/ej.17.1.200
Mayfield, A. B., Aguilar, C., Kolodziej, G., Enochs, I. C., & Manzello, D. P. (2021). Shotgun proteomic analysis of thermally challenged reef corals. Frontiers in Marine Science, 8. Doi: 10.3389/fmars.2021.660153
McMurdie, P. J., & Holmes, S. (2013). Phyloseq: an R package for reproducible interactive analysis and graphics of microbiome census data. PloS One, 8 (4), e61217. Doi: 10.1371/journal.pone.0061217
National Academies of Sciences, E., & Medicine. (2019). A research review of interventions to increase the persistence and resilience of coral reefs. Washington, DC: The National Academies Press.
Nobu, M. K., Narihiro, T., Rinke, C., Kamagata, Y., Tringe, S. G., Woyke, T., & Liu, W. T. (2015). Microbial dark matter ecogenomics reveals complex synergistic networks in a methanogenic bioreactor. ISME J, 9 (8), 1710-1722. Doi: 10.1038/ismej.2014.256
Oakley, C. A., Durand, E., Wilkinson, S. P., Peng, L., Weis, V. M., Grossman, A. R., & Davy, S. K. (2017). Thermal shock induces host proteostasis disruption and endoplasmic reticulum stress in the model symbiotic cnidarian Aiptasia. J Proteome Res, 16 (6), 2121-2134. Doi: 10.1021/acs.jproteome.6b00797
Oksanen, J., Blanchet, F. G., Friendly, M., Kindt, R., Legendre, P., McGlinn, D., . . . Solymos, P. (2020). Vegan: Community Ecology Package. R package version 2.5-6. 2019. In.
Palmer, C. V., & Traylor-Knowles, N. (2012). Towards an integrated network of coral immune mechanisms. Proceedings of the Royal Society B-Biological Sciences, 279 (1745), 4106-4114. Doi: 10.1098/rspb.2012.1477
Patro, R., Duggal, G., Love, M. I., Irizarry, R. A., & Kingsford, C. (2017). Salmon provides fast and bias-aware quantification of transcript expression. Nat Methods, 14 (4), 417-419. Doi: 10.1038/nmeth.4197
Poplin, R., Ruano-Rubio, V., DePristo, M. A., Fennell, T. J., Carneiro, M. O., Van der Auwera, G. A., . . . Banks, E. (2018). Scaling accurate genetic variant discovery to tens of thousands of samples. bioRxiv, 201178. Doi: 10.1101/201178
Prince, J. T. and Marcotte, E. M. (2006) Chromatographic Alignment of ESI-LC-MS Proteomics Data Sets by Ordered Bijective Interpolated Warping. Analytical Chemistry 78 (17), 6140-6152. DOI: 10.1021/ac0605344
Quast, C., Pruesse, E., Yilmaz, P., Gerken, J., Schweer, T., Yarza, P., . . . Glockner, F. O. (2013). The SILVA ribosomal RNA gene database project: improved data processing and web-based tools. Nucleic Acids Res, 41 (Database issue), D590-596. doi: 10.1093/nar/gks1219
Radecker, N., Raina, J. B., Pernice, M., Perna, G., Guagliardo, P., Kilburn, M. R., . . . Voolstra, C. R. (2018). Using Aiptasia as a model to study metabolic interactions in Cnidarian-Symbiodinium symbioses. Frontiers in Physiology, 9. doi: 10.3389/fphys.2018.00214
Rappsilber, J., Mann, M., & Ishihama, Y. (2007). Protocol for micro-purification, enrichment, pre-fractionation and storage of peptides for proteomics using StageTips. Nat Protoc, 2 (8), 1896-1906. doi: 10.1038/nprot.2007.261
Raser, J. M., & O'Shea, E. K. (2005). Noise in gene expression: origins, consequences, and control. Science, 309 (5743), 2010-2013. doi: 10.1126/science. 1105891
Revelle, W. (2022). psych: Procedures for Psychological, Psychometric, and Personality Research [Software].
Rohart, F., Gautier, B., Singh, A., & Le Cao, K. A. (2017). mixOmics: An R package for ‘omics feature selection and multiple data integration. PLOS Comput Biol, 13 (11), e1005752. doi: 10.1371/journal.pcbi. 1005752
Rosenberg, E., Koren, O., Reshef, L., Efrony, R., & Zilber-Rosenberg, I. (2007). The role of microorganisms in coral health, disease and evolution. Nat Rev Microbiol, 5 (5), 355-362. doi: 10.1038/nrmicro1635
Rothig, T., Costa, R. M., Simona, F., Baumgarten, S., Torres, A. F., Radhakrishnan, A., . . . Voolstra, C. R. (2016). Distinct bacterial communities associated with the coral model Aiptasia in aposymbiotic and symbiotic states with Symbiodinium. Frontiers in Marine Science, 3. doi: 10.3389/fmars.2016.00234
Shumaker, A., Putnam, H. M., Qiu, H., Price, D. C., Zelzion, E., Harel, A., . . . Bhattacharya, D. (2019). Genome analysis of the rice coral Montipora capitata. Sci Rep, 9 (1), 2571. doi: 10.1038/s41598-019-39274-3
Siebeck, U. E., Marshall, N. J., Kluter, A., & Hoegh-Guldberg, O. (2006). Monitoring coral bleaching using a colour reference card. Coral Reefs, 25 (3), 453-460. doi: 10.1007/s00338-006-0123-8
Stephens, T. G., Strand, E. L., Mohamed, A. R., Williams, A., Chiles, E. N., Su, X., . . . Putnam, H. M. (2021). Ploidy variation and its implications for reproduction and population dynamics in two sympatric Hawaiian coral species. bioRxiv, 2021.2011.2021.469467. doi: 10.1101/2021.11.21.469467
Struhl, K. (2007). Transcriptional noise and the fidelity of initiation by RNA polymerase II. Nat Struct Mol Biol, 14 (2), 103-105. doi: 10.1038/nsmb0207-103
Stuhr, M., Blank-Landeshammer, B., Reymond, C. E., Kollipara, L., Sickmann, A., Kucera, M., & Westphal, H. (2018). Disentangling thermal stress responses in a reef-calcifier and its photosymbionts by shotgun proteomics. Sci Rep, 8 (1), 3524. doi: 10.1038/s41598-018-21875-z
Szklarczyk, D., Gable, A. L., Lyon, D., Junge, A., Wyder, S., Huerta-Cepas, J., . . . Mering, C. V. (2019). STRING v11: protein-protein association networks with increased coverage, supporting functional discovery in genome-wide experimental datasets. Nucleic Acids Res, 47 (D1), D607-D613. doi: 10.1093/nar/gkyl131
Tarazona, S., Balzano-Nogueira, L., & Conesa, A. (2018). Chapter Eighteen-Multiomics data integration in time series experiments. In J. Jaumot, C. Bedia, & R. Tauler (Eds.), Comprehensive Analytical Chemistry (Vol. 82, pp. 505-532): Elsevier.
The, M., MacCoss, M. J., Noble, W. S., Käll, L. (2016). Fast and Accurate Protein False Discovery Rates on Large-Scale Proteomics Data Sets with Percolator 3.0. J Am Soc Mass Spectrom 27 (11), 1719-1727. doi: 10.1007/s13361-016-1460-7
Vogel, C., & Marcotte, E. M. (2012). Insights into the regulation of protein abundance from proteomic and transcriptomic analyses. Nature Reviews Genetics, 13 (4), 227-232. doi: 10.1038/nrg3185
Williams, A., Chiles, E. N., Conetta, D., Pathmanathan, J. S., Cleves, P. A., Putnam, H. M., . . . Bhattacharya, D. (2021a). Metabolomic shifts associated with heat stress in coral holobionts. Sci Adv, 7 (1). doi: 10.1126/sciadv.abd4210
Williams, A., Pathmanathan, J. S., Stephens, T. G., Su, X., Chiles, E. N., Conetta, D., . . . Bhattacharya, D. (2021b). Multi-omic characterization of the thermal stress phenome in the stony coral Montipora capitata. PeerJ, 9, e12335. doi: 10.7717/peerj.12335
Williams, A., Stephens, T. G., Shumaker, A., Bhattacharya, D. (2023). Peeling back the layers of coral holobiont multi-omics data. iScience, 26 (9), 107623. doi: 10.1016/j.isci.2023.107623
Wilson, D. F., & Matschinsky, F. M. (2021). Metabolic homeostasis in life as we know it: Its origin and thermodynamic basis. Front Physiol, 12, 658997. doi: 10.3389/fphys.2021.658997
Wright, J. J., Mewis, K., Hanson, N. W., Konwar, K. M., Maas, K. R., & Hallam, S. J. (2014). Genomic properties of Marine Group A bacteria indicate a role in the marine sulfur cycle. ISME J, 8 (2), 455-468. doi: 10.1038/ismej.2013.152
Xia, J., Psychogios, N., Young, N., & Wishart, D. S. (2009). MetaboAnalyst: a web server for metabolomic data analysis and interpretation. Nucleic Acids Res, 37 (Web Server issue), W652-660. doi: 10.1093/nar/gkp356
Zoccola, D., Morain, J., Pages, G., Caminiti-Segonds, N., Giuliano, S., Tambutte, S., & Allemand, D. (2017). Structural and functional analysis of coral Hypoxia Inducible Factor. PLOS One, 12 (11). doi: 10.1371/journal.pone.0186262

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A device for detecting environmental stress or an effect thereof in a sample from a marine invertebrate, wherein the device detects a biomarker.

2. The device of claim 1, wherein the environmental stress comprises thermal stress.

3. The device of claim 1, wherein the effect of environmental stress comprises at least one effect selected from the group comprising: reduced health, increased stress, reduced viability, reduced variation, reduced abundance, reduced proliferation, and reduced fecundity.

4. The device of claim 1, wherein the marine invertebrate comprises a holobiont.

5. The device of claim 1, wherein the marine invertebrate comprises coral.

6. The device of claim 1, wherein the device comprises a test strip or a lateral flow immunoassay (LFIA).

7. The device of claim 1, wherein the biomarker is detected at a level at least 2-fold higher or lower in the sample compared to a control.

8. The device of claim 1, wherein the biomarker is selected from the group comprising: phenylalanine-4-hydroxylase, glycine N-methyltransferase, glyoxylate/hydroxypyruvate reductase, 2′-5′-oligoadenylate synthetase, 4-hydroxyphenylpyruvate dioxygenase, and homogentisate 1,2-dioxygenase.

9. Use of the device of claim 1 for:
a) monitoring health of a marine invertebrate;
b) detecting environmental stress or an effect thereof in a marine invertebrate;
c) detecting viability, variation, abundance, proliferation, or fecundity of a marine invertebrate;
d) treating a marine invertebrate;
e) identifying a marine invertebrate for propagation or protection; or
f) a combination of any of the foregoing.

10. The use of claim 9, wherein treating a marine invertebrate comprises at least one intervention selected from the group comprising: prohibition of fishing, prohibition of swimming, and prohibition of boating.

11. A composition for use as a standard in detecting environmental stress or an effect thereof in a sample from a marine invertebrate, wherein the composition comprises one or more known amounts of one or more biomarkers.

12. The composition of claim 11, wherein the environmental stress comprises thermal stress.

13. The composition of claim 11, wherein the effect of environmental stress is at least one effect selected from the group comprising: reduced health, increased stress, reduced viability, reduced variation, reduced abundance, reduced proliferation, and reduced fecundity.

14. The composition of claim 11, wherein the marine invertebrate comprises a holobiont.

15. The composition of claim 11, wherein the marine invertebrate comprises coral.

16. The composition of claim 11, wherein at least one known amount of a biomarker is higher than a corresponding amount in the sample or in a control.

17. The composition of claim 11, wherein the biomarker comprises:
a) one or more proteins selected from the group comprising phenylalanine-4-hydroxylase, glycine N-methyltransferase, glyoxylate/hydroxypyruvate reductase, 2′-5′-oligoadenylate synthetase, 4-hydroxyphenylpyruvate dioxygenase, and homogentisate 1,2-dioxygenase;
b) one or more dipeptides selected from the group comprising arginine-glutamine, arginine-alanine, arginine-valine, and lysine-glutamine; or
c) a combination of any of the foregoing.

18. The composition of claim 11, wherein detecting the environmental stress or an effect thereof comprises:
a) measuring a first amount of a biomarker in a first sample from the marine invertebrate;
b) measuring a second amount of the biomarker in a control or in a second sample from the marine invertebrate; and
c) comparing the first and second amounts of the biomarker, wherein the second amount relative to the first amount indicates that the marine invertebrate is likely or predicted to be affected by environmental stress.

19. The composition of claim 18, wherein the first amount of the biomarker is at least 2-fold higher or lower than the second amount of the biomarker.

20. The composition of claim 18, wherein:
a) the first sample and the second sample are from the same individual, colony, population, or holobiont of the marine invertebrate;
b) the first sample and the second sample are from different individuals, colonies, populations, or holobionts of the marine invertebrate; or
c) the control is a standard.
